SEQUENCE LISTING (4 sequences)

SEQ ID NO: 1 - DNA, 5215 bp, Rattus norvegicus
  misc_feature 1..5215: note = Description of sequence: Rattus norvegicus outer membrane protein (OMP25) mRNA
  source 1..5215: mol_type = genomic DNA
  gene 456..1076: note = Rattus norvegicus outer membrane protein gene
  misc_feature 963..1076: note = c-terminal domain
gagagcccagccttctccctggctcctatttaagcatcaggcaaagaccgtgggtcccagcaacagcagtgaggaggcacttggcacttcgggcaggtggtagccgctgtgcctggcaccttggcatcctgcctgggctgcacgtggcctggcacccatacagctcctgattgctgcggctggaaggagcctgtgattctgcgtgagtgtggggtccttgggtatacagtcagccgaggtagaggtgggctggcctaggactgtacacttctctaggagctgggcttaactaaaacagaagagattgcggttagacatcaagaggaactcagacccgtgggaaagaaccaggacctgaggcaaaggcgagatttgggtggtggacgggggacagggagactcttcacagtcctcacaggacctacacaggggcctatggtctcagacctgggatcatgttcctgagggggtataagagagaggggcaggtcaggaagcataggaagtcagttgcacatgggggagggttgtggggaaactgtaaaagctatgctggggaggcggcacggagcttgattcaccttcacctgcgccgggcacccgctgaccccgggtttccgcccggagagcagtcagatatgaacggacgggtggattatttagtctccgaggaagagatcaacctgaccagaggaccctcggggctgggcttcaacatcgtcggtgggacagatcaacagtatgtctccaatgacagtggcatctacgtcagccgcatcaaagaggatggggctgcggcccgggatgggcggctccaggagggtgataagatcctctcggtaaatggccaagacctgaagaacctgttgcaccaagatgccgtagacctcttccgtaatgcaggatatgccgtgtccctgagagtgcagcacaggttaccagtgcagaatggacctatagttcatcgaggcgacggagagccgagtggagttcctgtagctgtggtgctgctgccagtgtttgcccttaccctggtagcagtttgggccttcgtgagataccgaaagcagctctgagatgcctgttgtcttccagtgtgtccgatgagctaactatctctctcactcaccatctcgacatcctcccctagtcttccttcccacatagccaacacatgatttaaagtgactgcttatcacccgaaaccttgctgttcaaaatctccaagacttcacattctaatggaagagtaaagagattatttgaagaaagctgggggtggggagagccttgcttagaataaatgagaagttacatattttactagaactgccaataaaaattcagctatcagccaaagaggagaagcttgctcttcctgtctccatggacgacaccttttgcttagctggtgtgctttgaaggctagctgtgctatgtgaaaggaggagctgattttttaaatactttttcttgggaagtatttgtggcctttaatttgtaactatatacttagatgcctatattggacataggcgaatgaatttttttcttttcttagaaaagaaaatacatatatacattaacacacacacacacacacacacacacacagcataaaactgatgccttatggagagttaaagaggtgagaaaactactggttcttggatttctagtggacaagttttgggaactagggggtcatattcctttatatataatcaaaactcatattaaagaatgagttctggattgtaaagagaacttactttttccacttgtctgtaagtctttgtccacaagttaaaaacatacacagtcctaagggctgatcataactgaaacacttcaaaaattattggcagagaaggtataacggggatggaaaagttagctttaaaagagaatgctcagtcagtggcattaggaaaataatgactgaagcaattagttgaaaactgtagaacaactagtgttccagggtagctacagtgatgtgggaaattgtggcaaagcagttcttctcaagtcaatttgtttaaaagactttggggtgtaaagatgagtcctgacagtctctgaatgaccttagtgactgttgtactatgtgaaaggatccattggcatggagatgagaagtaggagggccaaataaaccatcaagacaactgaaaaaagatcatacctcagggtggcacttgaaccctcaaagtgctcaccaactttcagtttctagtatcaggcaaagcctaagggaagggactgctttcctggcagggtggaccccaaatcagcctgcttctgatagagttacgatgagaaggcccaaattgatgtaggctttctttttgtgtatttttcccaaagctataagcactagttttggttttaataaggtaatggaccattttttcagaaaaaggggaaggaataaggtttgtttgtttatagagggtgcaaggaagtttagtgagcatttctgaggaaagcctgagcacacagagtagattaactatgccagagaatagccagggatattggggggtggggggtagtggagctgtcctgtttatgtagcatcaaagttggttttagagtactgaggatagttgaaccaagcctaattttttttttcactttaacaaaataatttccgtcttttcaataaattggccagactattttgacctaagattataaagagttttcctgacaacctagcagtcacttaacagatgtcgattgcacattagttttgtcaagacacctttataacaaagggaaatgcacacacttcatccaaaaggacattgtggttacagttaggaagaaaacacttaagatggaattatccatgtaagtgtaagttgtttatattacagacaacaaaagaaaatagctcccaagctttgagcactgaagatctcagaggaaggactccttaggtgccaatcgtctagcaagcatgcatacataggggacaagttaagagtcttaccgggttgagtaagaaaaggaaattattgccatgcgagacagagcacgatgcagcaatgtggaggatgagcagacagctagcatgttgtaaggagctcagaacgggagagccttcctgcagtggcatcgggggagaatgcaaaggaaagtcttgaacagtccagtgagcctttgcctctggataaactgttgatacagggagacatttgctttaaacagtcttcacataaccaagccattcagtacttcttgaaactgacttcataacaggagtcattgtaagttccacagaaagcaagacgtatgtatttcagttcttgtcttgaccagcagcactccggaggcccagtgtccggtgccctccttgtatctgaagcaggggtaacagctctgctgtgggcctgtttccctctagtatttacctcaaggcttggaaatgtattttgaaagaccttcagtcaaacgaagtaaagcaaatgtcaagaaggataaaccactgttgtgtgttgatgtgtggtgtattgtgatgtaaataaaaaatatcgttgaagttttactttgtaaagattctggtaacacttgggtctccttgggaaagaaacaacacttgaccaaagcatttaagttttaggcaaattccataatatttcactcttagttataaaattatcatacatgagatgttgaggatctatatagattattcagattttatattccttatagattgactcaaatattccttaaaagattatatatgatgctcctatggcataaattgtaatgtttaatgttttgaaaattggttataggtattttttaagttcccataaagtgtttcttgtatttgtcattgtgaattatatgtaattctaatagatattgtttgctttagtcatttttgtctccttttcctatagtttcctgtgtgagtgattccatgtaattaattggacagtctatccacaggcaacaaatagaagtagtatttgtaattgcagcctgtgccagggactgtgttagactgattagtaacactatgaatcacccgagatcttaagtattgctataaagagaagactgaagatgagagagtaaagtattttcctgggattatatagttggtaaaaaagaatcatgatttaaactctggactttggataaaagtatcagtaattttcctcattgctagcagtcctcatgcaaggtctctggactaaagcgatgtattacctgcaaatgcttgagtttcatcccatacttatggcgtgaggtgagtgtggcagccagcatctctgtcctcctaggtaactggttagtgctgaacctcgagattcaccactgtcaggagctgtctctgcttagaatcacctggacagctggctgtcaacccacgttaagtaagaacatgtgggactagaattcagtcaacaataaaagctttcatcaggtggctaaaatgtgcagactctgtttgtactggcatgttggccatcactcacagatgcaactctagccagaatcaagaatggggcagatccagagcccagctcgctgagcttctgccccagatttcagaggcagagtaagaagtggaacagtgttttcctcccaggtgctcaggacaggctggtcaacttccagtcacatatgccatttagtaagtgcttcggcctgtgcagccattagtaaccaaatcatgggatagtgaatctaaaatactcttggataaaatttgtattagaattaaaaaaaaatgagggtcagaaaggtgaactaactccaacttagatgcccctcccctttattttggtctcaaatctgttttaatgtgtccacaacattaatcaaaaactgtattttttttttttcggagctggggactgaacccagggccttgcgcttgctaggcaagcgctctaccactgagctaaatccccaacccccaaaactgtattttaaaaatcagttcactttcatctttttttccttgggttttcagtaaagaccagaaggactgacagacagctagagaggttgtgtagacatccagatggggagagtgcattggacaatctgagaaatctattcaggtgctgcctaattccattagctttaagttagatacaagctgtttgtagtattttgggttttattgtgcaaataaacaatagaggcctttagtcccagcgttaggatgcagagatccatttct

SEQ ID NO: 2 - DNA, 8590 bp, synthetic construct
  misc_feature 1..8590: note = Description of sequence: DNA Vector vector ID: VB220328-1304ynx
  source 1..8590: mol_type = other DNA
  regulatory 1..229: note = Promoter Rous sarcoma virus; regulatory_class = promoter
  misc_feature 230..410: note = long terminal repeat
  misc_feature 521..565: note = HIV-1 packaging signal, named Psi
  misc_feature 1075..1308: note = signal, named RRE, HIV-1 Rev response element
  misc_feature 1803..1920: note = Central polypurine tract, cPPT
  regulatory 1950..2418: note = Promoter Human synapsin I promoter, named: SYN1; regulatory_class = promoter
  regulatory 2443..2448: note = Kozak translation initiation sequence; regulatory_class = other
  gene 2449..3069: note = coding sequence: rtOMP25C-ter Lenti W
  regulatory 3094..3681: note = linker, named IRES Recruits ribosome to initiate translation; regulatory_class = ribosome_binding_site
  gene 3682..4401: note = coding sequence: EmGFP
  regulatory 4431..5028: note = Woodchuck hepatitis virus posttranscriptional regulatory element (WPRE); regulatory_class = enhancer
  misc_feature 5110..5344: note = long terminal repeat, named: 3' LTR-delta-U3
  polyA_site 5417..5551: note = polyadenylation signal, SV40 early pA
  gene 6505..7365: note = AmpiciIIin
  regulatory 7536..8124: regulatory_class = uORF
  misc_feature 2605: note = cutting site for restriction enzyme, SmaI
  misc_feature 2795: note = cutting site for restriction enzyme, SmaI
  misc_feature 1700: note = cutting site for restriction enzyme, AvaI
  misc_feature 2603: note = cutting site for restriction enzyme, AvaI
  misc_feature 2692: note = cutting site for restriction enzyme, AvaI
  misc_feature 2793: note = cutting site for restriction enzyme, AvaI
  misc_feature 1798: note = cutting site for restriction enzyme, NheI
  misc_feature 2603: note = cutting site for restriction enzyme, XmaI
  misc_feature 2793: note = cutting site for restriction enzyme, XmaI
  misc_feature 3573: note = cutting site for restriction enzyme, ApaLI
  misc_feature 4645: note = cutting site for restriction enzyme, ApaLI
  misc_feature 6621: note = cutting site for restriction enzyme, ApaLI
  misc_feature 7867: note = cutting site for restriction enzyme, ApaLI
  misc_feature 2603: note = cutting site for restriction enzyme pair, ApaLI+XmaI
  misc_feature 2793: note = cutting site for restriction enzyme pair, ApaLI+XmaI
  misc_feature 3573: note = cutting site for restriction enzyme pair, ApaLI+XmaI
  misc_feature 4645: note = cutting site for restriction enzyme pair, ApaLI+XmaI
  misc_feature 6621: note = cutting site for restriction enzyme pair, ApaLI+XmaI
  misc_feature 7867: note = cutting site for restriction enzyme pair, ApaLI+XmaI
  misc_feature 2603: note = cutting site for restriction enzyme pair, ApaLI+SmaI
  misc_feature 2793: note = cutting site for restriction enzyme pair, ApaLI+SmaI
  misc_feature 3573: note = cutting site for restriction enzyme pair, ApaLI+SmaI
  misc_feature 4645: note = cutting site for restriction enzyme pair, ApaLI+SmaI
  misc_feature 6621: note = cutting site for restriction enzyme pair, ApaLI+SmaI
  misc_feature 7867: note = cutting site for restriction enzyme pair, ApaLI+SmaI
  misc_feature 1700: note = cutting site for restriction enzyme pair, ApaLI+AvaI
  misc_feature 2603: note = cutting site for restriction enzyme pair, ApaLI+AvaI
  misc_feature 2692: note = cutting site for restriction enzyme pair, ApaLI+AvaI
  misc_feature 2793: note = cutting site for restriction enzyme pair, ApaLI+AvaI
  misc_feature 3573: note = cutting site for restriction enzyme pair, ApaLI+AvaI
  misc_feature 4645: note = cutting site for restriction enzyme pair, ApaLI+AvaI
  misc_feature 6621: note = cutting site for restriction enzyme pair, ApaLI+AvaI
  misc_feature 7867: note = cutting site for restriction enzyme pair, ApaLI+AvaI
  misc_feature 1798: note = cutting site for restriction enzyme pair, ApaLI+NheI
  misc_feature 3573: note = cutting site for restriction enzyme pair, ApaLI+NheI
  misc_feature 4645: note = cutting site for restriction enzyme pair, ApaLI+NheI
  misc_feature 6621: note = cutting site for restriction enzyme pair, ApaLI+NheI
  misc_feature 7867: note = cutting site for restriction enzyme pair, ApaLI+NheI
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtatcaactttgtatagaaaagttgctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagcaagtttgtacaaaaaagcaggctgccaccatgttcctgagggggtataagagagaggggcaggtcaggaagcataggaagtcagttgcacatgggggagggttgtggggaaactgtaaaagctatgctggggaggcggcacggagcttgattcaccttcacctgcgccgggcacccgctgaccccgggtttccgcccggagagcagtcagatatgaacggacgggtggattatttagtctccgaggaagagatcaacctgaccagaggaccctcggggctgggcttcaacatcgtcggtgggacagatcaacagtatgtctccaatgacagtggcatctacgtcagccgcatcaaagaggatggggctgcggcccgggatgggcggctccaggagggtgataagatcctctcggtaaatggccaagacctgaagaacctgttgcaccaagatgccgtagacctcttccgtaatgcaggatatgccgtgtccctgagagtgcagcacaggttaccagtgcagaatggacctatagttcatcgaggcgacggagagccgagtggagttcctgtagctgtggtgctgctgccagtgtttgcccttaccctggtagcagtttgggccttcgtgagataccgaaagcagctctgaacccagctttcttgtacaaagtgggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacaactttattatacatagttgatcaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 3 - DNA, 1113 bp, synthetic construct
  misc_feature 1..1113: note = Description of sequence: Chimera coding sequence: ATAT1 fused to the mitochondrially targeting OMP25 domain
  source 1..1113: mol_type = other DNA
  gene 1..999: note = Homo sapiens alpha tubulin acetyltransferase 1 (ATAT1), transcript variant 2, mRNA
  misc_feature 1000..1113: note = Rattus norvegicus synaptojanin 2 binding protein (Synj2bp), mRNA
atggagttcccgttcgatgtggacgcgctgttcccggagcggatcacggtgctggaccagcacctgaggcccccagcccgccgacccggaaccacaacgccggcccgtgttgatctacagcagcaaattatgaccattatagatgaactgggcaaggcttctgccaaggcccagaatctttccgctcctatcactagtgcatcaaggatgcagagtaaccgccatgttgtttatattctcaaagacagttcagcccgaccggctggaaaaggagccattattggtttcatcaaagttggatacaagaagctctttgtactggatgatcgtgaggctcataatgaggtagaaccactttgcatcctggacttttacatccatgagtctgtgcaacgccatggccatgggcgagaactcttccagtatatgttgcagaaggagcgagtggaaccgcaccaactggcaattgaccgaccctcacagaagctgctgaaattcctgaataagcactacaatctggagaccacagtcccacaggtgaacaactttgtgatctttgaaggcttctttgcccatcaacatcggccccctgctccctctctgagggcaactcgacactctcgtgctgctgcagtcgatcccacgcccgctgctccagcaaggaagctgccacccaagagagcagagggagacatcaagccatactcctctagtgaccgagaatttctgaaggtagctgtggagcctccttggcccctaaacagggcccctcgccgcgccacacctccagcccacccacccccccgctccagcagcctgggaaactcaccagaacgaggtcccctccgcccctttgtgccagagcaggagctgctgcgttccttgcgcctctgccccccacaccctaccgcccgccttctgttggctgctgaccctgggggcagcccagctcaacgtcgtcgcaccagctcccttccccgctctgaggagagtcgataccatcgaggcgacggagagccgagtggagttcctgtagctgtggtgctgctgccagtgtttgcccttaccctggtagcagtttgggccttcgtgagataccgaaagcagctctga

SEQ ID NO: 4 - DNA, 9082 bp, synthetic construct
  misc_feature 1..9082: note = Description of sequence: Vector: Vector ID VB220329-1381pbx
  source 1..9082: mol_type = other DNA
  regulatory 1..229: note = RSV promoter, Promoter Rous sarcoma virus enhancer/promoter; regulatory_class = promoter
  misc_feature 230..410: note = long terminal repeat, from: Truncated HIV-1 5' , named: 5' LTR-delta-U3
  misc_feature 521..565: note = HIV-1 packaging signal, named: psi
  misc_feature 1075..1308: note = HIV-1 Rev response element, named: RRE
  misc_feature 1803..1920: note = Central polypurine tract, named: cPPT
  regulatory 1950..2418: note = Human synapsin I promoter, named: SYN1; regulatory_class = promoter
  regulatory 2443..2448: note = Kozak translation initiation sequence; regulatory_class = other
  gene 2449..3561: note = open read frame, named: hATAT1(2)rtOMP25C-terLenti W
  regulatory 3586..4173: note = Linker: Encephalomyocarditis virus internal ribosome entry site, named: IRES; regulatory_class = ribosome_binding_site
  gene 4174..4893: note = open read frame, named: Emerald green fluorescent protein(emGFP)
  regulatory 4923..5520: note = Woodchuck hepatitis virus posttranscriptional regulatory element (WPRE); regulatory_class = promoter
  misc_feature 5602..5836: note = long terminal repeat, from: Truncated HIV-1 3' , named: 3' LTR-delta-U3
  polyA_site 5909..6043: note = Simian virus 40 early polyadenylation signal, named: SV40 early pA
  gene 6997..7857: note = AmpiciIIin resistance gene
  regulatory 8028..8616: note = pUC origin of replication; regulatory_class = uORF
  misc_feature 6689: note = cutting site for restriction enzyme, DrdI
  misc_feature 8571: note = cutting site for restriction enzyme, DrdI
  misc_feature 1922: note = cutting site for restriction enzyme, SpeI
  misc_feature 2542: note = cutting site for restriction enzyme, SpeI
  misc_feature 4065: note = cutting site for restriction enzyme, ApaLI
  misc_feature 5137: note = cutting site for restriction enzyme, ApaLI
  misc_feature 7113: note = cutting site for restriction enzyme, ApaLI
  misc_feature 8359: note = cutting site for restriction enzyme, ApaLI
  misc_feature 5522: note = cutting site for restriction enzyme, EcoRI
  misc_feature 3951: note = cutting site for restriction enzyme, DraIII
  misc_feature 6645: note = cutting site for restriction enzyme, DraIII
  misc_feature 1922: note = cutting site for restriction enzyme pair, ApaLI+SpeI
  misc_feature 2542: note = cutting site for restriction enzyme pair, ApaLI+SpeI
  misc_feature 4065: note = cutting site for restriction enzyme pair, ApaLI+SpeI
  misc_feature 5137: note = cutting site for restriction enzyme pair, ApaLI+SpeI
  misc_feature 7113: note = cutting site for restriction enzyme pair, ApaLI+SpeI
  misc_feature 8359: note = cutting site for restriction enzyme pair, ApaLI+SpeI
  misc_feature 4065: note = cutting site for restriction enzyme pair, ApaLI+DrdI
  misc_feature 5137: note = cutting site for restriction enzyme pair, ApaLI+DrdI
  misc_feature 6689: note = cutting site for restriction enzyme pair, ApaLI+DrdI
  misc_feature 7113: note = cutting site for restriction enzyme pair, ApaLI+DrdI
  misc_feature 8359: note = cutting site for restriction enzyme pair, ApaLI+DrdI
  misc_feature 8571: note = cutting site for restriction enzyme pair, ApaLI+DrdI
  misc_feature 4065: note = cutting site for restriction enzyme pair, ApaLI+EcoRI
  misc_feature 5137: note = cutting site for restriction enzyme pair, ApaLI+EcoRI
  misc_feature 5522: note = cutting site for restriction enzyme pair, ApaLI+EcoRI
  misc_feature 7113: note = cutting site for restriction enzyme pair, ApaLI+EcoRI
  misc_feature 8359: note = cutting site for restriction enzyme pair, ApaLI+EcoRI
  misc_feature 3951: note = cutting site for restriction enzyme pair, ApaLI+DraIII
  misc_feature 4065: note = cutting site for restriction enzyme pair, ApaLI+DraIII
  misc_feature 5137: note = cutting site for restriction enzyme pair, ApaLI+DraIII
  misc_feature 6645: note = cutting site for restriction enzyme pair, ApaLI+DraIII
  misc_feature 7113: note = cutting site for restriction enzyme pair, ApaLI+DraIII
  misc_feature 8359: note = cutting site for restriction enzyme pair, ApaLI+DraIII
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtatcaactttgtatagaaaagttgctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagcaagtttgtacaaaaaagcaggctgccaccatggagttcccgttcgatgtggacgcgctgttcccggagcggatcacggtgctggaccagcacctgaggcccccagcccgccgacccggaaccacaacgccggcccgtgttgatctacagcagcaaattatgaccattatagatgaactgggcaaggcttctgccaaggcccagaatctttccgctcctatcactagtgcatcaaggatgcagagtaaccgccatgttgtttatattctcaaagacagttcagcccgaccggctggaaaaggagccattattggtttcatcaaagttggatacaagaagctctttgtactggatgatcgtgaggctcataatgaggtagaaccactttgcatcctggacttttacatccatgagtctgtgcaacgccatggccatgggcgagaactcttccagtatatgttgcagaaggagcgagtggaaccgcaccaactggcaattgaccgaccctcacagaagctgctgaaattcctgaataagcactacaatctggagaccacagtcccacaggtgaacaactttgtgatctttgaaggcttctttgcccatcaacatcggccccctgctccctctctgagggcaactcgacactctcgtgctgctgcagtcgatcccacgcccgctgctccagcaaggaagctgccacccaagagagcagagggagacatcaagccatactcctctagtgaccgagaatttctgaaggtagctgtggagcctccttggcccctaaacagggcccctcgccgcgccacacctccagcccacccacccccccgctccagcagcctgggaaactcaccagaacgaggtcccctccgcccctttgtgccagagcaggagctgctgcgttccttgcgcctctgccccccacaccctaccgcccgccttctgttggctgctgaccctgggggcagcccagctcaacgtcgtcgcaccagctcccttccccgctctgaggagagtcgataccatcgaggcgacggagagccgagtggagttcctgtagctgtggtgctgctgccagtgtttgcccttaccctggtagcagtttgggccttcgtgagataccgaaagcagctctgaacccagctttcttgtacaaagtgggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacaactttattatacatagttgatcaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

(19) United States
(12) Patent Application Publication
(10) Pub. No.: US 2026/0183429 A1
(43) Pub. Date: Jul. 2, 2026

(54) COMPOSITIONS FOR MODULATING ACETYLATED TUBULIN LEVELS AND METHODS THEREOF

(71) Applicant: THE TRUSTEES OF COLUMBIA UNIVERSITY IN THE CITY OF NEW YORK, New York, NY (US)

(72) Inventors: Atul KUMAR, Fort Lee, NJ (US); Francesca BARTOLINI, New York, NY (US)

(73) Assignee: THE TRUSTEES OF COLUMBIA UNIVERSITY IN THE CITY OF NEW YORK, New York, NY (US)

(21) Appl. No.: 19/547,316

(22) Filed: Feb. 23, 2026

Related U.S. Application Data
(63) Continuation of application No. PCT/US2024/043814, filed on Aug. 26, 2024.
(60) Provisional application No. 63/578,569, filed on Aug. 24, 2023.

(51) Int. Cl. A61K 48/00; A61K 38/00; A61P 25/02; C12N 9/10; C12N 15/86

(52) U.S. Cl. CPC A61K 48/0058; A61P 25/02; C12N 9/1025; C12N 15/86; A61K 38/00; C07K 2319/07; C12N 2740/15043; C12Y 203/01108

(57) ABSTRACT

The present disclosure relates to compositions for increasing acetylated alpha-tubulin levels in peripheral neuronal cells. The present disclosure also relates to the use of tubulin deacetylase inhibitors, or activators of tubulin acetylation for increasing acetylated tubulin levels in the cells. The present disclosure also relates to methods for assessing risk of developing peripheral neuropathy by assessing the levels of acetylated alpha-tubulin. The present disclosure further relates to methods for treating a subject suffering from a peripheral neuropathy using the compositions described herein.

### CROSS-REFERENCE TO RELATED APPLICATIONS

This application is a continuation of International Patent Application No. PCT/US2024/043814, filed on Aug. 26, 2024, which claims priority to U.S. Provisional Application Ser. No. 63/578,569, filed on Aug. 24, 2023, the contents of each of which are incorporated by reference in their entireties, and to each of which priority is claimed.

### STATEMENT REGARDING FEDERALLY SPONSORED RESEARCH

This invention was made with government support under NS120076 and AG050658, awarded by the National Institute of Health. The government has certain rights in the invention.

### SEQUENCE LISTING

The present specification makes reference to a Sequence Listing (submitted electronically as a xml file named “070050.6982_ST26.xml” on Feb. 23, 2026). The 070050.6982_ST26.xml file was generated on Feb. 23, 2026 and is 75,929 bytes in size.

The entire contents of the Sequence Listing are hereby incorporated by reference.

### 1. INTRODUCTION

The present disclosure relates to acetylated tubulin, including compositions comprising inhibitors and activators of tubulin acetylation. The present disclosure also relates to methods for detecting risk of a subject suffering from axonal degradation. The present disclosure further relates to methods for treating peripheral neuropathy in a subject.

### 2. BACKGROUND

Familial peripheral neuropathy reminiscent of diseases like Charcot-Marie-Tooth (CMT) type 2A disease can cause sensory loss due to degeneration of long peripheral axons. The mitochondrial fusion protein mitofusin-2 (MFN2), a large GTPase residing in the outer mitochondrial membrane (OMM) can regulate mitochondrial fusion and its tethering to the endoplasmic reticulum (ER). Certain inherited dominant mutations in MFN2 are common causes of CMT. Many MFN2 mutations can affect the amino terminal GTPase domain, with disease onset in the first two years of life and an aggressive clinical course. MFN2 plays a role in mitochondria dynamics, including regulation of fusion, motility, and ER/mitochondria contacts.

Defects in mitochondria dynamics can be associated with CMT pathogenesis, including CMT caused by MFN2 mutations. A fusion and tethering independent role for MFN2 in regulating mitochondrial axonal transport can occur, where loss of MFN2, or MFN2 disease mutants selectively alter mitochondrial axonal motility and distribution. In addition, MFN2 deficiency in human spinal motor neurons can interfere with mitochondrial transport while reducing the expression of kinesin and dynein motors, which can further contribute to impaired mitochondrial motility. MFN2 interacts with mammalian miro (miro1/miro2) and milton/TRAK (OIP106/GRIF1) proteins, members of the molecular machinery that links mitochondria to kinesin motors. Taken together, MFN2 can directly influence mitochondrial positioning, and loss of this function in CMT can contribute to the degeneration of long axons, which are sensitive to failures in meeting local energy and calcium buffering demands. This is consistent with the observation that many genes mutated in predominantly axonal forms of CMT have roles in mitochondrial motility, suggesting that impaired mitochondrial transport can be a common mechanism of CMT pathogenesis.

Acetylated microtubules play a role in the regulation of mitochondria dynamics. In sensory neurons, acetylated tubulin is an essential component of the mammalian mechano-transduction machinery due to its regulation of cellular stiffness and tubulin can affect transient receptor potential (TRP) channel activity. Loss of acetylated tubulin is a neuropathological feature of CMT2A mouse models and vincristine-induced toxicity. Compositions and methods for enhancing tubulin acetylation can therefore be beneficial for restoring axonal integrity, and myelination of toxic and genetically inherited forms of peripheral neuropathy. Acetylation of α-tubulin is predominantly regulated by tubulin N-acetyltransferase 1 (α-TAT 1 or ATAT1) and histone deacetylase 6 (HDAC6). However, while tubulin is the only target of ATAT1, HDAC6 can deacetylate several substrates in addition to tubulin. The field is also lacking in superior methods for detecting risk of axonal degradation and targeted methods for treating peripheral neuropathy.

### 3. SUMMARY

The presently disclosed subject matter provides methods for determining whether a subject is at risk of developing peripheral neuropathy. In an example, a method includes obtaining a test sample from a subject who has not been diagnosed with suffering from peripheral neuropathy and determining in the sample, a level of acetylated tubulin. A lower level of acetylated tubulin in the test sample compared to a control level indicates that the subject is at an increased risk of developing peripheral neuropathy.

The presently disclosed subject matter also provides methods of determining whether a subject has peripheral neuropathy. In an example, a method includes obtaining a test sample from the subject, and determining in the sample, a level of acetylated tubulin. A lower expression level of acetylated tubulin compared to a control level indicates that the subject has peripheral neuropathy in need of treatment. It can also be used to monitor the progression of the neuropathy as acetylated tubulin has been shown to best correlate with the severity of the pathology in a longitudinal study of 45 CMT1A patients (see abstract attached to the email).

In certain embodiments, the sample is a biopsy sample. In certain embodiments, the sample is a biopsy of a peripheral nerve. In certain embodiments, the sample is a dermal biopsy sample. In certain embodiments, the sample comprises one or more peripheral neuronal cells. In certain embodiments, the peripheral neuronal cell is a sensory neuronal cell, a motor neuronal cell, or a peripheral blood mononuclear cell (PBMC).

In certain embodiments, the level of acetylated tubulin is determined by optimized ELISA methods, immunofluorescence, western blotting, mass spectrometry, or a combination thereof.

In certain embodiments, the method comprises determining a control level of acetylated tubulin from a control sample and comparing this value with the level of acetylated tubulin in the test sample. In certain embodiments, the method comprises comparing the level of acetylated tubulin in the test sample with a control level of acetylated tubulin obtained from a reference standard.

In certain embodiments, the method further comprises treating the subject having peripheral neuropathy or the subject identified as at increased risk of developing peripheral neuropathy. In certain embodiments, treating comprises administering to the subject, a therapeutically effective amount of a compound that increases tubulin acetylation.

In certain embodiments, the compound that increases tubulin acetylation is an inhibitor of tubulin deacetylation. In certain embodiments, the compound that increases tubulin acetylation is an activator of tubulin acetylation. In certain embodiments, the inhibitor of tubulin deacetylation is a histone deacetylase (HDAC) inhibitor. In certain embodiments, the HDAC inhibitor is a HDAC6 inhibitor. In certain embodiments, the HDAC inhibitor is selected from trichostatin A (TSA), Tubacin, N-hydroxy-N′-phenyl-octanediamide, and TN-301, or a combination thereof. To these days, several HDAC6 inhibitors are under trial for neuropathic disease_see Miralinc. In certain embodiments, the activator of tubulin acetylation is an α-tubulin acetyltransferase 1 (ATAT1) activator or ectopic expression of ATAT1 or mitochondrially targeted ATAT1 by gene therapy methods.

The presently disclosed subject matter also provides methods of treating a subject suffering from a disease associated with peripheral neuropathy. In an example, a method includes administering to the subject a composition comprising a therapeutically effective amount of a compound that increases tubulin acetylation.

In certain embodiments, the disease associated with peripheral neuropathy is diabetes, hypothyroidism, kidney disease, liver disease, cancer, Charcot-Marie-Tooth (CMT) disease, Guillain-Barré syndrome, autoimmune disease, rheumatoid arthritis, Lyme disease and lupus.

In certain embodiments, the compound that increases tubulin acetylation is an inhibitor of tubulin deacetylation. In certain embodiments, the compound that increases tubulin acetylation is an activator of tubulin acetylation. In certain embodiments, the inhibitor of tubulin deacetylation is a histone deacetylase (HDAC) inhibitor. In certain embodiments, the HDAC inhibitor is a HDAC6 inhibitor. In certain embodiments, the HDAC inhibitor is selected from the group consisting of trichostatin A (TSA), Tubacin, N-hydroxy-N′-phenyl-octanediamide, and TN-301, (there are many others) or a combination thereof. In certain embodiments, the activator of tubulin acetylation is an α-tubulin acetyltransferase 1 (ATAT1) activator or ectopic expression of ATAT1 or mitochondrially targeted ATAT1 by gene therapy methods.

In certain embodiments, the inhibitor of tubulin deacetylation is in a therapeutically effective concentration from about 2 nM to about 1000 nM. In certain embodiments, the inhibitor of tubulin deacetylation is in a therapeutically effective concentration of 10 nM. In certain embodiments, the inhibitor of tubulin deacetylation is in a therapeutically effective concentration from about 5 μM to about 1000 μM in the medicament. In certain embodiments, the inhibitor of tubulin deacetylation is in a therapeutically effective concentration of 20 μM.

The presently disclosed subject matter also provides methods of treating a subject. In an example, a method includes determining a level of acetylated tubulin in a sample obtained from a subject, identifying the subject as suffering from a peripheral neuropathy when the level of acetylated tubulin in the sample from the subject is lower compared to a control level, and administering to the identified subject, a composition comprising an inhibitor of tubulin deacetylation.

The presently disclosed subject matter also provides methods of treating a subject. In an example, a method includes determining a level of acetylated tubulin in a sample obtained from a subject, identifying the subject as suffering from a peripheral neuropathy when the level of acetylated tubulin in the sample from the subject is lower compared to a control level, and administering to the identified subject, a composition comprising an activator of tubulin acetylation.

The presently disclosed subject matter also provides methods of treating a subject. In an example, a method includes determining a level of acetylated tubulin in a sample obtained from a subject, identifying the subject as suffering from a peripheral neuropathy when the level of acetylated tubulin in the sample from the subject is lower compared to a control level, and administering to the identified subject, a composition comprising an inhibitor of tubulin deacetylation and an activator of tubulin acetylation.

In certain embodiments, the sample is a biopsy sample. In certain embodiments, the sample is a biopsy of a peripheral nerve. In certain embodiments, the sample is a dermal biopsy sample. In certain embodiments, the sample comprises one or more peripheral neuronal cells. In certain embodiments, the peripheral neuronal cell is a sensory neuronal cell, a motor neuronal cell, or a PBMC.

In certain embodiments, the level of acetylated tubulin is determined by optimized ELISA methods, immunofluorescence, western blotting, mass spectrometry, or a combination thereof.

In certain embodiments, the method comprises determining a control level of acetylated tubulin from a control sample and comparing this value with the level of acetylated tubulin in the test sample. In certain embodiments, the method comprises comparing the level of acetylated tubulin in the test sample with a control level of acetylated tubulin obtained from a reference standard.

In certain embodiments, the composition comprises an inhibitor of tubulin deacetylation. In certain embodiments, the composition comprises an activator of tubulin acetylation. In certain embodiments, the inhibitor of tubulin deacetylation is a histone deacetylase (HDAC) inhibitor. In certain embodiments, the HDAC inhibitor is a HDAC6 inhibitor. In certain embodiments, the HDAC inhibitor is selected from the group consisting of trichostatin A (TSA), Tubacin, N-hydroxy-N′-phenyl-octanediamide, and TN-301, (more) or a combination thereof. In certain embodiments, the activator of tubulin acetylation is an α-tubulin acetyltransferase 1 (ATAT1) activator.

In certain embodiments, the inhibitor of tubulin deacetylation is in a therapeutically effective concentration from about 2 nM to about 1000 nM. In certain embodiments, the inhibitor of tubulin deacetylation is in a therapeutically effective concentration of 10 nM.

In certain embodiments, the inhibitor of tubulin deacetylation is in a therapeutically effective concentration from about 5 μM to about 1000 μM in the medicament. In certain embodiments, the inhibitor of tubulin deacetylation is in a therapeutically effective concentration of 20 μM.

The presently disclosed subject matter also provides methods for treating a subject suffering from a disease associated with peripheral neuropathy including, administering to the subject, a composition comprising a nucleic acid encoding a fusion polypeptide. In an example, the nucleic acid encoding a fusion polypeptide includes a nucleic acid sequence encoding a mitochondria targeting sequence operably linked to a nucleic acid sequence encoding a polypeptide sequence that increases levels of acetylated tubulin.

The presently disclosed subject matter also provides methods for treating a subject suffering from a disease associated with peripheral neuropathy. In an example, a method includes determining a level of acetylated tubulin in a sample obtained from a subject, identifying the subject as suffering from a peripheral neuropathy when the level of acetylated tubulin in the sample from the subject is lower compared to a control level, and administering to the identified subject, a composition comprising a nucleic acid encoding a fusion polypeptide. In an example, the nucleic acid encoding a fusion polypeptide includes a nucleic acid sequence encoding a mitochondria targeting sequence operably linked to a nucleic acid sequence encoding a polypeptide sequence that increases levels of acetylated tubulin at sites of mitochondria contacts with microtubules.

In certain embodiments, the peripheral neuropathy is associated with diabetes, hypothyroidism, kidney disease, liver disease, cancer, Charcot-Marie-Tooth (CMT) disease, Guillain-Barré syndrome, autoimmune disease, rheumatoid arthritis, Lyme disease and lupus.

In certain embodiments, the method comprises determining a control level of acetylated tubulin from a control sample and comparing this value with the level of acetylated tubulin in the test sample. In certain embodiments, the method comprises comparing the level of acetylated tubulin in the test sample with a control level of acetylated tubulin obtained from a reference standard.

In certain embodiments, the level of acetylated tubulin is determined by immunofluorescence, western blotting, mass spectrometry, or a combination thereof.

In certain embodiments, the tubulin is α-tubulin.

In certain embodiments, the sample is a biopsy sample. In certain embodiments, the sample is a biopsy of a peripheral nerve. In certain embodiments, the sample is a dermal biopsy sample.

In certain embodiments, the sample comprises one or more peripheral neuronal cells. In certain embodiments, the peripheral neuronal cell is a sensory neuronal cell, a motor neuronal cell, or a PBMC.

In certain embodiments, the nucleic acid encoding the fusion polypeptide further comprises a promoter sequence disposed upstream to the fusion polypeptide sequence. In certain embodiments, the promoter is a human synapsin I promoter (SYN1).

In certain embodiments, the mitochondrial targeting sequence is fused to the C-terminus of the polypeptide sequence.

In certain embodiments, the mitochondria targeting sequence is outer membrane protein 25 (OMP25).

In certain embodiments, the polypeptide sequence that increases levels of acetylated tubulin is α-tubulin acetyltransferase 1 (ATAT1).

In certain embodiments, the mitochondria targeting sequence is an OMP25 sequence, the polypeptide sequence that increases levels of acetylated tubulin is ATAT1, and the nucleic acid encoding the fusion polypeptide comprises the sequence set forth in SEQ ID NO.: 3.

In certain embodiments, the nucleic acid encoding the fusion polypeptide is further comprised in a viral vector. In certain embodiments, the viral vector is selected from the group consisting of adeno-associated virus (AAV), recombinant adenoviruses (rAV), and lentivirus (LV). In certain embodiments, the viral vector is a lentivirus. In certain embodiments, the viral vector comprising the nucleic acid encoding the fusion polypeptide comprises a sequence set forth in SEQ ID NO.: 4.

The presently disclosed subject matter also provides a method of treating a subject suffering from a disease associated with neuropathy. In an example, the method includes administering to the subject, a composition comprising a nucleic acid encoding a fusion polypeptide comprising a nucleic acid sequence encoding outer membrane protein 25 (OMP25), a nucleic acid sequence encoding α-tubulin acetyltransferase 1 (ATAT1) operably linked to OMP25 and a human synapsin I promoter (SYN1) sequence disposed upstream to the nucleic acid encoding the fusion polypeptide sequence, wherein the OMP25 peptide is fused to the C-terminus of ATAT1 and wherein the nucleic acid encoding the fusion polypeptide comprises the sequence set forth in SEQ ID NO.: 3.

In certain embodiments, the composition is a pharmaceutical composition that further comprises a pharmaceutically acceptable carrier.

In certain embodiments, the neuropathy is a peripheral neuropathy. In certain embodiments, the neuropathy is associated with diabetes, hypothyroidism, kidney disease, liver disease, cancer, Charcot-Marie-Tooth (CMT) disease, Guillain-Barré syndrome, autoimmune disease, rheumatoid arthritis, Lyme disease and lupus.

In certain embodiments, the composition is administered to a peripheral nerve in the subject. In certain embodiments, the composition is administered to a neuronal cell in the subject. In certain embodiments, the composition is administered to a peripheral neuronal cell in the subject. In certain embodiments, the composition is administered to a sensory neuron in the subject.

The presently disclosed subject matter also provides methods for identifying a compound as effective for treatment of peripheral neuropathy. In an example, a method includes obtaining a sample, determining an original level of acetylated tubulin in the sample, contacting the sample with the compound, and determining in the sample an updated level of acetylated tubulin. A higher level of acetylated tubulin compared to the original level indicates that the compound is effective for treatment of peripheral neuropathy.

In certain embodiments, the sample is a biopsy sample. In certain embodiments, the sample is a biopsy of a peripheral nerve. In certain embodiments, the sample is a dermal biopsy sample. In certain embodiments, the sample comprises one or more peripheral neuronal cells. In certain embodiments, the peripheral neuronal cell is a sensory neuronal cell, or a motor neuronal cell.

In certain embodiments, the acetylated tubulin is acetylated α-tubulin. In certain embodiments, the level of acetylated tubulin is determined by optimized ELISA methods, immunofluorescence, western blotting, mass spectrometry, or a combination thereof.

In certain embodiments, the compound is an inhibitor of tubulin deacetylation. In certain embodiments, the compound that increases tubulin acetylation is an activator of tubulin acetylation. In certain embodiments, the inhibitor of tubulin deacetylation is a histone deacetylase (HDAC) inhibitor. In certain embodiments, the HDAC inhibitor is a HDAC6 inhibitor. In certain embodiments, the HDAC inhibitor is selected from the group consisting of trichostatin A (TSA), Tubacin, N-hydroxy-N′-phenyl-octanediamide, and TN-301, (many more) or a combination thereof. In certain embodiments, the activator of tubulin acetylation is an α-tubulin acetyltransferase 1 (ATAT1) activator.

The presently disclosed subject matter also provides a composition comprising an inhibitor of tubulin deacetylation, and/or an activator of tubulin acetylation.

In certain embodiments, the composition is a pharmaceutical composition that further comprises a pharmaceutically acceptable carrier.

In certain embodiments, the inhibitor of tubulin deacetylation is a histone deacetylase (HDAC) inhibitor. In certain embodiments, the HDAC inhibitor is a HDAC6 inhibitor. In certain embodiments, the HDAC inhibitor is selected from the group consisting of trichostatin A (TSA), Tubacin, N-hydroxy-N′-phenyl-octanediamide, and TN-301, or a combination thereof. In certain embodiments, the activator of tubulin acetylation is an α-tubulin acetyltransferase 1 (ATAT1) activator. In certain embodiments, the tubulin is α-tubulin.

In certain embodiments, the inhibitor of tubulin deacetylation is in a therapeutically effective concentration from about 2 nM to about 1000 nM. In certain embodiments, the inhibitor of tubulin deacetylation is in a therapeutically effective concentration of 10 nM. In certain embodiments, the inhibitor of tubulin deacetylation is in a therapeutically effective concentration from about 5 μM to about 1000 μM in the medicament. In certain embodiments, the inhibitor of tubulin deacetylation is in a therapeutically effective concentration of 20 μM.

The presently disclosed subject matter also provides a composition comprising a nucleic acid encoding a fusion polypeptide comprising a nucleic acid sequence encoding a mitochondria targeting sequence operably linked to a nucleic acid sequence encoding a polypeptide sequence that increases levels of acetylated tubulin preferentially at sites of mitochondria contacts with microtubules.

In certain embodiments, the composition is a pharmaceutical composition that further comprises a pharmaceutically acceptable carrier.

In certain embodiments, the nucleic acid encoding the fusion polypeptide further comprises a promoter sequence disposed upstream to the fusion polypeptide sequence. In certain embodiments, the promoter is a human synapsin I promoter (SYN1).

In certain embodiments, the mitochondrial targeting sequence is fused to the C-terminus of the polypeptide sequence.

In certain embodiments, the mitochondria targeting sequence is outer membrane protein 25 (OMP25).

In certain embodiments, the polypeptide sequence that increases levels of acetylated tubulin is α-tubulin acetyltransferase 1 (ATAT1).

In certain embodiments, the mitochondria targeting sequence is an OMP25 sequence, the polypeptide sequence that increases levels of acetylated tubulin is ATAT1, and the nucleic acid encoding the fusion polypeptide comprises the sequence set forth in SEQ ID NO.: 3.

The presently disclosed subject matter also provides a viral vector comprising a nucleic acid encoding a fusion polypeptide comprising a nucleic acid sequence encoding a mitochondria targeting sequence operably linked to a nucleic acid sequence encoding a polypeptide sequence that increases levels of acetylated tubulin at mitochondria contacts with microtubules.

In certain embodiments, the mitochondria targeting sequence is an OMP25 sequence, the polypeptide sequence that increases levels of acetylated tubulin is ATAT1, and the nucleic acid encoding the fusion polypeptide comprises the sequence set forth in SEQ ID NO.: 3.

In certain embodiments, the viral vector is selected from the group consisting of adeno-associated virus (AAV), recombinant adenoviruses (rAV), and lentivirus (LV). In certain embodiments, the viral vector is a lentivirus. In certain embodiments, the viral vector comprising the nucleic acid encoding the fusion polypeptide comprises a sequence set forth in SEQ ID NO.: 4.

The presently disclosed subject matter also provides for use of a compound that increases tubulin acetylation, in the manufacture of a medicament for the treatment of peripheral neuropathy in a subject.

In certain embodiments, the compound that increases tubulin acetylation is an inhibitor of tubulin deacetylation. In certain embodiments, the compound that increases tubulin acetylation is an activator of tubulin acetylation. In certain embodiments, the inhibitor of tubulin deacetylation is a histone deacetylase (HDAC) inhibitor. In certain embodiments, the HDAC inhibitor is a HDAC6 inhibitor. In certain embodiments, the HDAC inhibitor is selected from the group consisting of trichostatin A (TSA), Tubacin, N-hydroxy-N′-phenyl-octanediamide, and TN-301, more or a combination thereof. In certain embodiments, the activator of tubulin acetylation is an α-tubulin acetyltransferase 1 (ATAT1) activator.

In certain embodiments, the inhibitor of tubulin deacetylation is in a concentration from about 2 nM to about 1000 nM. In certain embodiments, the inhibitor of tubulin deacetylation is in a concentration from about 5 μM to about 1000 μM in the medicament.

The presently disclosed subject matter also provides a kit for identifying a compound effective for the treatment of peripheral neuropathy. In an example, the kit includes a specific binding partner for acetylated tubulin, a sample comprising cells, and instructions for identifying the compound.

In certain embodiments, the specific binding partner is an antibody or a fragment thereof. In certain embodiments, the antibody fragment is selected from the group consisting of: a Fab, a Fab′, an F(ab′)₂, an scFv, an (scFv)₂, a single-chain antibody, a VHH antibody and a minibody.

In certain embodiments, the acetylated tubulin is acetylated α-tubulin. In certain embodiments, the level of acetylated tubulin is determined by ELISA, immunofluorescence, western blotting, mass spectrometry, or a combination thereof.

In certain embodiments, the cell is a sensory neuronal cell, or a motor neuronal cell or a Schwann cell. In certain embodiments, the cell is a peripheral neuronal cell. In certain embodiments, the peripheral neuronal cell is a sensory neuronal cell, or a motor neuronal cell or a Schwann cell.

### 4. BRIEF DESCRIPTION OF THE DRAWINGS

FIGS. 1A-1I show regulation of α-tubulin acetylation, microtubule (MT) dynamics and stability by MFN2. FIG. 1A shows immunoblot analysis of whole cell lysates from wild type (WT) and MFN2 KO mouse embryonic fibroblasts (MEF) for acetylated tubulin (Acet), detyrosinated tubulin (deTyr), mitofusin 2 (MFN2), and loading control (GAPDH). FIG. 1B shows representative confocal immunofluorescence images of WT and MFN2 KO MEFs for the indicated proteins. FIG. 1C shows a quantitation of acetylated tubulin in MFN2 KO MEFs compared with WT MEF calculated from immunofluorescence signal intensity. FIG. 1D shows a quantitation of detyrosinated tubulin in MFN2 KO MEFs compared with WT MEF calculated from immunofluorescence intensity data from FIG. 1A. FIG. 1E shows the ratio of acetylated tubulin to detyrosinated tubulin in WT and MFN2 KO MEFs calculated from immunofluorescence intensity data. FIG. 1F shows GTP tubulin staining in WT and MFN2 KO MEFs using hMB11 antibody. FIG. 1G shows quantification of GTP-tubulin (MB11) immunofluorescence signal intensity associated with MTs in WT and MFN2 KO MEFs. FIG. 1H shows representative confocal immunofluorescence images of residual MT staining (DM1A) in WT and MFN2 KO extracted MEFs. FIG. 1I shows quantification of residual DM1A tubulin levels in WT and MFN2 KO MEFs treated as in FIG. 1H.

FIGS. 2A-2H show restoration of acetylated tubulin levels and mitochondria association to MTs following HDAC inhibition. FIG. 2A shows immunoblot analysis of Acet, deTyr, total α-tubulin (DM1A) and MFN2 in whole cell lysates from wild type (WT) and MFN2 KO incubated with control vehicle or 10 nM of trichostatin A (TSA) for 6 h and 12 h. FIG. 2B shows quantification of acetylated tubulin relative to control levels (%) in WT and MFN2 KO MEFs treated as described in FIG. 2A. FIG. 2C show representative immunofluorescence staining of Acet, deTyr and tyrosinated (Tyr) tubulin in WT and MFN2 KO MEFs incubated with control vehicle or TSA for 6 h. FIG. 2D show quantification of acetylated to tyrosinated tubulin immunofluorescence signal ratio measured in individual cells treated as in FIG. 2C. FIG. 2E show representative immunofluorescence staining of mitochondria and tyrosinated tubulin (Tub) in MFN2 KO MEFs incubated with control vehicle or 10 nM of TSA for 6 h. FIG. 2F shows quantification of mitochondria associated with MTs (identified by the bulk tubulin marker tyrosinated tubulin) using Mander's coefficient. FIG. 2G show immunoblot analysis of MFN2, Acetylated-miro1 (Lys105 MIRO1) and total miro1 (MIRO1) in whole cell lysates from WT and MFN2 KO MEFs. FIG. 2H shows quantification of total MIRO1 levels in M_FN2 KO MEFs relative to WT MEF.

FIGS. 3A-3L show rescue of mitochondria motility and cholesterol esterification following HDAC6 inhibition in MFN2 KO MEFs.

FIG. 3A show immunofluorescence of WT and MFN2 KO cells stained with mitoTracker Red and treated with TSA (10 nM) or vehicle control for 6 h. FIG. 3B shows quantification of mitochondrial displacement velocity analyzed from movies acquired for 3 min (1f/2s) in cells treated as in FIG. 3A. FIG. 3C shows quantification of relative distribution of mitochondria (#) to the geometrical cell center in cells treated as in FIG. 3A. FIG. 3D shows quantification of mitochondria aspect ratio (length/width) in cells treated as in FIG. 3A. FIG. 3E shows quantification of mitochondria fusion using mitoDendra expression in cells treated as in FIG. 3A. FIG. 3F shows quantification of mitochondrial displacement velocity analyzed from movies acquired for 3 min (1f/2s) in cells treated with tubacin (20 μM). FIG. 3G shows quantification of relative distribution of mitochondria (#) to the geometrical cell center in cells treated with tubacin (20 μM). FIG. 3H shows quantification of mitochondria aspect ratio (length/width) in cells treated with tubacin (20 μM). FIG. 3I shows quantification of mitochondria fusion using mitoDendra expression in cells treated with tubacin or control vehicle for 6 h. FIG. 3J shows a heatmap representation of changes in the indicated lipid classes in MFN2 KO MEFs treated with vehicle control or TSA (10 nM). Key: Ceramide (Cer), dihydroceramide (dhCer), sphingomyelin (SM), dehydrosphingomyelin (dhSM), monosialodihexosylganglioside (GM3). Bis(monoacylglycerol)phosphate (BMP), acylated phosphatidylglycerol (Acyl-PG), Lysophosphatidylcholine (LPC), Lysophosphatidylcholine plasmalogen (LPCe), Lysophosphatidylethanolamine (LPE), Lysophosphatidylethanolamine plasmalogen (LPEp). FIG. 3K shows a heatmap representation of changes in the indicated lipid classes in MFN2 KO MEFs treated with vehicle control or TSA (10 nM). Key: free cholesterol (FC), cholesteryl esters (CE), phosphatidic acid (PA), phosphatidylcholine (PC), phosphatidylcholine plasmalogen (PCe), phosphatidylethanolamine (PE); phosphatidylethanolamine plasmalogen (Pep), phosphatidylserine (PS), phosphatidylinositol (PI), phosphatidylglycerol (PG). FIG. 3L shows phospholipid synthesis and transfer between ER and mitochondria in WT and MFN2 KO MEFs treated with vehicle control or TSA (10 nM) for 6 h, as evaluated by incorporation of ³H-Ser into ³H-PtdSer (PS) and (H)³H-PtdEtn (PE) after 2 h and 4 h, and expressed as % of the average value measured in the controls.

FIGS. 4A-4H show restoration of acetylated tubulin levels in Iqgap1 KO MEFs following HDAC inhibition. FIG. 4A is an immunoblot of whole cell lysates from WT and Iqgap1 KO MEFs for the indicated proteins. FIG. 4B shows representative immunofluorescence staining of Acet, deTyr and Tyr tubulins in WT and Iqgap1 KO MEFs. FIG. 4C shows quantification of deTyr tubulin signal in Iqgap1 KO MEFs relative to WT levels. FIG. 4D shows quantification of Acet tubulin signal in Iqgap1 KO MEFs relative to WT levels. FIG. 4E is an immunoblot for the indicated proteins in whole cell lysates from WT and Iqgap1 KO MEFs incubated with 10 nM of the HDAC inhibitor trichostatin A (TSA) or vehicle control for 6 h or 12 h. FIG. 4F Quantification of acetylated tubulin (Acet) levels relative to vehicle control (%) in WT and Iqgap1 KO MEFs incubated with 10 nM TSA for 6 or 12 h.

FIG. 4G shows representative immunofluorescence staining of WT and Iqgap1 KO MfFs incubated with 10 nM of TSA or vehicle control for 6 h. FIG. 4H shows quantification of acetylated (Acet) to tyrosinated (Tyr) tubulin immunofluorescence signal ratio measured in WT and Iqgap1 KO MEFs as in FIG. 4G.

FIGS. 5A-5I shows rescue of mitochondria dynamics in Iqgap1 KO MEFs following restoration of tubulin acetylation. FIG. 5A shows immunofluorescence of mitoTracker Red stained WT and Iqgap1 KO MEFs incubated with vehicle control, or 10 nM trichostatin A (ISA) for 6 h. FIG. 5B shows quantification of mitochondria morphology, calculated as ratio of length/width in cells treated as in FIG. 5A. FIG. 5C shows relative distribution of mitochondria from the cell center in MEFs treated as in FIG. 5A. FIG. 5D shows mitochondrial displacement velocity near the cell center and periphery in MEFs treated as in FIG. 5A. FIG. 5E is a schematic of the enzymes involved in the α-tubulin detyrosination/retyrosination cycle. Note that detyrosination of α-tubulin occurs on MTs while α-tubulin retyrosination occurs on the soluble tubulin heterodimer. Key: tubulin carboxypeptidases (TCPs), detyrosinated α-tubulin (deTyr), tyrosinated α-tubulin (Tyr), tubulin tyrosine ligase (TTL). FIG. 5F is a representative immunoblot of whole cell lysates from WT and Iqgap1 KO MEFs depleted of tubulin tyrosine ligase (TTL) expression by shRNA transfection for 3 days. FIG. 5G shows mitochondria morphology as calculated as a ratio of length/width in cells depleted of TTL expression. FIG. 5H shows relative distribution of mitochondria from the cell center in MIFs depleted of TTL expression. FIG. 5I shows mitochondrial displacement velocity near the cell center and periphery in MEFs depleted of TTL expression.

FIGS. 6A-6H shows MFN2 regulation of HDAC6 levels and localization of ATAT1. FIG. 6A shows immunoblot analysis of HDAC6 and ATAT1 levels in whole cell lysates from WT and MFN2 KO MEFs. FIG. 6B shows quantification of expression level of HDAC6 in MFN2 KG MEFs relative to WT MEFs. FIG. 6C shows quantification of expression level of ATAT1 in MFN2 KO MEFs relative to WT MEFs. FIG. 6D shows representative immunofluorescence staining of ATAT1 and HDAC6 in WT and MFN2 KO MEFs. FIG. 6E shows mitotic index assessed by nuclear DAPI staining in WT and MFN2 KO MEFs. FIG. 6F shows immunoblot analysis of cytosolic and nuclear envelope/endoplasmic reticulum (ER) fractions from WT and MFN2 KO MEF lysates. Key: T==:total lysate; C=cytoplasmic fraction; NER=nuclear ER fraction. FIG. 6G shows quantification of ATAT1 levels in cytoplasmic versus NER fraction expressed as ratio of intensity values from analysis as in FIG. 6F. FIG. 6H shows representative immunoblots of mitochondrial and cytoplasmic fraction from WT and MFN2 KO MEF lysates. FIG. 6I shows quantification of ATAT1 level in mitochondria versus cytoplasmic fraction expressed as ratio of intensity values.

FIGS. 7A-7M shows localization of ATAT1 to mitochondria outer membranes by MFN2. FIG. 7A shows Airyscan confocal images (single plane) of mitochondria (TOMM20) and ATAT1 in WT and MFN2 KO MEFs. White arrows show localization of ATAT1 relative to TOMM20. FIG. 7B shows line scan analysis of mitochondria and ATAT1 localization in WT MEF from selected regions from FIG. 7A. FIG. 7C shows line scan analysis of mitochondria and ATAT1 localization in MFN2 KO MEF from selected regions from FIG. 7A. FIG. 7D shows quantification of the localization of ATAT1 in the mitochondria from FIG. 7A by Mander's correlation coefficient. FIG. 7E shows number of ATAT1 foci on μm of mitochondria from FIG. 7A. FIG. 7F shows Airyscan confocal images (single plane) of ATAT1 and MFN2 localization at mitochondria (Mito tracker red) in MEFs. White arrow heads show localization of MFN2 and ATAT1 on mitochondria. FIG. 7G shows line scan analysis of MFN2 and ATAT1 localization at mitochondria in WT cells. FIG. 7H shows quantification of ATAT1 signal localization at mitochondria and the fraction of this signal that co-localizes with MFN2. FIG. 7I shows immunofluorescence analysis of MFN2 and ATAT1 PLA signal in WT and MFN2 KO MEFs. FIG. 7J shows quantification of PLA puncta per cell in WT and MFN2 KO MEFs. FIG. 7K shows interaction between ATAT1 and MFN2, detected by immunoprecipitation (IP) followed by immunoblot analysis with the indicated antibodies (upper panel) and interaction between endogenous ATAT1 and transfected Myc-MFN2 WT, detected in HEK293T cells (lower panel). FIG. 7L shows co-immunoprecipitation analysis of HEK293T cells co-transfected with Myc-MFN2 WT and Flag-ATAT1 WT or Flag-ATAT1 (1-242) or Flag-ATAT1 (1-286) to access interaction between MFN2 and ATAT1 in WT or mutants MEFs. FIG. 7M shows normalized MFN2 to ATAT1 signal ratio from FIG. 7L.

FIGS. 8A-8C shows association of ATAT1 with mitochondria outer membranes at sites of contact with nicked MTs. FIG. 8A shows mitochondrial networks (mitoTracker Red stained) and MFN2/ATAT1 co-localizing puncta in a WT MEF cell imaged by Airyscan confocal microscopy. FIG. 8B shows an Airyscan confocal image showing ATAT1 punctuate localization at mitochondria (mitoTracker Red stained) and tyrosinated tubulin (tub) in a WT MEF cell. The white arrowhead shows a nick in the MT lattice. FIG. 8C interaction between transfected Flag-ATAT1 and MFN2, but not the outer mitochondrial protein TOMM20 or the inner mitochondrial protein COX4 in HEK293T cells as detected by immunoprecipitation (IP) of FLAG-ATAT1 followed by immunoblot with the indicated antibodies.

FIGS. 9A-9M illustrates that regulation of α-tubulin acetylation by MFN2 by MFN2 mutations and shared by MFN1 FIG. 9A shows IP analysis of HEK293T cells co-transfected with Flag-ATAT1 and Myc-MFN2 WT or Myc-MFN2 R94W. FIG. 9B shows IP analysis of HEK293T cells co-transfected with Flag-ATAT1 and Myc-MFN2 WT or Myc-MFN2 T105M. FIG. 9C shows quantification of ratio of the MFN2 signal to the ATAT1 signal from FIGS. 9A and 9B. FIG. 9D shows representative confocal immunofluorescence images showing overexpression of Myc-MFN2 in WT, Myc-MFN2 R94W, Myc-MFN2 T105M MEF cells. FIG. 9E shows Mander's coefficient analysis for WT MEF cells co-transfected with Myc-MFN2 WT, Myc-MFN2 R94W and Myc-MFN2 T105M. FIG. 9E shows immunoblot analysis of acetylated tubulin levels in MFN2 KO cells co-transfected with Myc-MFN2 WT, Myc-MFN2 R94W and Myc-MFN2 T105M. FIG. 9G shows quantification of acetylated tubulin levels expressed as % of control levels from 3 independent experiments as in FIG. 9F. FIG. 9H shows immunoblot of acetylated tubulin levels in MFN1 overexpressing WT and MFN2 KO cells. FIG. 9I shows quantification of acetylated tubulin levels expressed as % of control levels from 4 independent experiments from FIG. 9H. FIG. 9J shows interactions between ATAT1 or its truncated mutants (1-242; 1-286) and endogenous Miro2 or Kif5c, detected in HEK293T cells transfected with Flag-ATAT1 WT or its truncated mutants. FIG. 9K shows ratio of the Miro2 and Kif5c signal to the ATAT1 signal from the data in FIG. J. FIG. 9L shows interaction between Flag-ATAT1 and endogenous Miro2 or Kif5c, detected in HEK293T cells in presence or absence of Myc-MFN2 WT, Myc-MFN2 R94W and Myc-MFN2 T105M. FIG. 9M shows the ratio of the Miro2 and Kif5c signal to the ATAT1 signal from FIG. 9L is plotted.

FIGS. 10A-10K show effect of MFN1-dependent regulation of acetylated tubulin levels on HDAC6 protein levels and loss of MFN2 expression suppresses endogenous ATAT1/miro binding in MEFs. FIG. 10A shows representative confocal immunofluorescence images showing mitochondria (mitodsRed) in WT MEF cells overexpressing Myc-MFN2 WT, Myc-MFN2 R94W, Myc-MFN2 T105M. FIG. 10B shows quantification of HDAC6 levels in WT and MFN2 KO MEFs upon overexpression of WT and mutant MFN2. FIG. 10 C shows quantification of HDAC6 levels in WT and MFN2 KO MEFs upon overexpression of MNF1.

FIG. 10D shows immunoblot of acetylated tubulin (Acet tub), HDAC6 and ATAT1 levels in WT and MFN1 KG cells. FIG. 10E shows quantification of Acet tub levels in WT and MFN1 KO cells from FIG. 10D. FIG. 10F shows quantification of ATAT1 levels in WT and MFN1 KO cells from FIG. 10D. FIG. 10G shows quantification of HDAC6 levels in WT and MFN1 KO cells from FIG. 10D. FIG. 10H shows representative immunofluorescence staining (maximum projections of acetylated (Acet), detyrosinated (deTyr) and tyrosinated (Tyr) MTs in WT and MFN1 KO MEFs. FIG. 10I shows quantification of acetylated tubulin signal in MFN11 KO MEFs relative to WT levels from images in FIG. 10H. FIG. 10J shows representative confocal immunofluorescence images of Miro1 and ATAT1 PLA puncta in WT and MFN2 KO MEFs. FIG. 10K shows quantification of PLA puncta per cell in WT and MFN2 KO MEFs from FIG. 10J.

FIGS. 11A-11L show regulation of α-tubulin acetylation in sensory neurons in vitro and in vivo by MFN2. FIG. 11A shows immunoblots of MFN2, acetylated (Acet) and detyrosinated tubulin (deTyr) levels in adult DRG neurons (14DIV) silenced of MFN2 expression at 7 DIV. FIG. 11B shows quantification of acetylated tubulin staining in MFN2 depleted DRG neurons relative to control neurons infected with shNC (non-coding shRNA). FIG. 11C shows representative confocal immunofluorescence images of late third-instar larvae in control RNAi and MarfKD. Multi-dendritic neuron driver 109(2)80 Gal4 driver was used to label Drosophila larval somatosensory neurons (CD8-GFP). FIG. 11D shows an illustration of WT Marf and MarfRNAi late third-instar larvae. FIG. 11E shows quantification of acetylated tubulin in somatosensory neurons from FIG. 11C. Two different strategies were used, Marf KD-1 (BL 67158) and KD-2 (BL 31157). FIG. 11F shows images of representative fields showing dissociated adult DRG neurons (14 DIV) treated as in FIG. 11A, fixed and immunostained with mouse anti-neurofilament (2H3-s) antibody. FIG. 11G shows quantification of number of retraction bulbs per field. FIG. 11H shows the degree of axonal degeneration, calculated as the ratio between fragmented axonal area and total axonal area. FIG. 11I shows immunoblot of MFN2, acetylated tubulin (Acet) levels in control (shNC) and MFN2 (shMFN2) silenced DRG neurons incubated with 10 nM of the HDAC6 inhibitor TSA or vehicle control for 6 h prior to lysis. FIG. 11J shows representative widefield immunofluorescence images of DRG neurons treated as in FIG. 11I. FIG. 11K shows quantification of number of retraction bulbs per field in DRG neurons treated as in FIG. 11I. FIG. 11L shows degree of axonal degeneration in DRG neurons treated as in FIG. 11I.

FIGS. 12A-12N show that ATAT1 localization to mitochondria is dependent on MIFN2 expression in primary adult DRG neurons and loss of acetylated tubulin precedes axonal degeneration in MIFN2 KD DRG neurons. FIG. 12A shows a representative Airyscan confocal image (single plane) of ATAT1 and MFN2 at mitochondria (mitoTracker Red) in adult DRG neurons (14 days). FIG. 12B shows a linescan analysis of ATAT1 and MFN2 signals from the white line shown in the zoomed image. FIG. 12C shows quantification of MFN2 and ATAT1 co-localization as in FIG. 12A by Mander's correlation coefficient. FIG. 12D shows Airyscan confocal images (single plane) of TOMM20 and ATAT1 signals in shNC and shMFN2 KD adult DRG neurons (14 days) infected at 7 DIV. FIGS. 12E-12H shows line scan analysis at proximal and distal axonal segments in DRG neurons treated as in FIG. 12D. FIG. 12I shows Mander's coefficient analysis of TOMM20 and ATAT1 signals from images of neurons treated as in FIG. 12D. FIG. 12J shows neurofilament staining of shNC and shMWN2 KD DRG neurons infected for either 3 or 5 days starting at 7 DIV. FIG. 12K shows degeneration index of axons in shNC and shMFN2 silenced DRG neurons treated as in FIG. 12J. FIG. 12L shows immunoblots of MFN2 and acetylated tubulin (Acet) levels in DRG neurons treated as in FIG. 12J. FIG. 12M shows quantification of acetylated tubulin (Acet tub) levels at 3 days, from immunoblot analysis as shown in FIG. 12L. FIG. 12N shows quantification of acetylated tubulin (Acet tub) levels at 5 days from immunoblot analysis as shown in FIG. 12L.

FIGS. 13A-13F show that ATAT1, but not mutant MIFN2, rescues axonal degeneration in MFN2 depleted DRG neurons. FIG. 13A shows neurofilament staining of shMFN2 K.D DRG neurons at 14DIV overexpressing the mitochondrial targeting domain of OMP25 alone or the fusion protein ATAT1-OMP25 by lentiviral infection for 5 days. FIG. 13B shows quantification of degree of axonal degeneration in shNC and shMFN2 KD DRG neurons as in FIG. 13A. FIG. 13C shows neurofilament staining of shNC DRG neurons at 7DIV overexpressing R94W or T105M MFN2 mutants by retroviral infection for 5 days prior to treatment with 10 nM TSA for 6 h. FIG. 13D shows quantification of degree of axonal degeneration in adult DRG neurons (14DIV) as in FIG. 13C. FIG. 13E shows neurofilament staining of shMFN2 KD DRG neurons overexpressing R94W or T105M MFN2 mutants by retroviral infection for 5 days prior to treatment with 10 nM TSA for 6 h. FIG. 13F shows quantification of degree of axonal degeneration in adult DRG neurons (14DIV) as in FIG. 13E.

FIGS. 14A-14F shows that HDAC6 levels are not rescued by mutant MFN2 but remain unaffected by MFN1 expression. FIG. 14A shows quantification of HDAC6 levels in WT and MFN2 KO MEFs upon overexpression of WT and mutant MFN2. FIG. 14B shows quantification of HDAC6 levels in WT and MFN2 KO MEFs upon overexpression of MFN1. FIG. 14C shows immunoblots of Acet tub, HDAC6 and ATAT1 levels in WT and MIFN1 KO cells. FIG. 14D shows quantification of Acet tub levels in WT and MFN1 KO cells as in FIG. 14C. FIG. 14E shows quantification of HDAC6 levels in WT and MFN1 KO cells as in FIG. 14C. FIG. 14D shows quantification of ATAT1 levels in WT and MFN1 KO cells as in FIG. 14C.

### 5. DETAILED DESCRIPTION

The presently disclosed subject matter relates to compositions for increasing acetylated α-tubulin levels in cells. The presently disclosed subject matter also relates to compositions for inhibiting tubulin deacetylation using tubulin deacetylase inhibitors and/or tubulin acetylation activators. The presently disclosed subject matter also provides methods for determining whether a subject is suffering from peripheral neuropathy, and if so how fast it progresses, or is at risk of developing peripheral neuropathy, and methods of treating a subject suffering from a disease associated with peripheral neuropathy. The presently disclosed subject matter also provides methods for determining whether a compound is effective for treatment of peripheral neuropathy. The presently disclosed subject matter also provides kits for identifying compounds that are effective for the treatment of peripheral neuropathy.

The subject matter of the present disclosure is described with reference to the figures. It should be understood that numerous specific details, relationships, and methods are set forth in this Detailed Description, Examples, and accompanying Figures to provide a more complete understanding of the subject matter disclosed herein. For purposes of clarity of disclosure and not by way of limitation, the detailed description is divided into the following subsections:
(i) Definitions
(ii) Deacetylase Inhibitors
(iii) Acetylase Activators
(iv) Nucleic Acid Compositions
(v) Pharmaceutical Compositions
(vi) Methods
(vii) Kits
(viii) Examples

### 5.1. Definitions

The terms used in this specification generally have their ordinary meanings in the art, within the context of the present disclosure and in the specific context where each term is used. Certain terms are discussed below, or elsewhere in the specification, to provide additional guidance to the practitioner in describing the formulations and methods of the disclosed subject matter and how to make and use them.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

The terms “homology” or “homologous thereto,” as used herein, refer to the degree of homology between nucleic acid or amino acid sequences as determined using methods known in the art, for example, but not limited to, software such as BLAST or FASTA.

As used herein, an “Inhibitor” refers to a compound or molecule (e.g., small molecule, peptide, peptidomimetic, natural compound, siRNA, anti-sense nucleic acid, aptamer, or antibody) that interferes with (e.g., reduces, prevents, decreases, suppresses, eliminates or blocks) the activity, function, expression and/or generation of a protein or pathway. An inhibitor can be any compound or molecule that changes any activity of a named protein (molecule, any molecule involved with the named molecule or a named associated molecule), such as α-tubulin, or interferes with the interaction of a named protein, e.g., α-tubulin, or acetylated α-tubulin, with signaling partners or binding partners. Inhibitors also include molecules that indirectly regulate the biological activity of a named protein, e.g., α-tubulin, or acetylated α-tubulin, by intercepting upstream signaling molecules. In certain embodiments, the inhibitor can include molecules that inhibit, minimize and/or reduce the generation and/or production of a named protein such as α-tubulin, or acetylated α-tubulin e.g., by interfering with the activity and/or function of the enzymes that generate and/or produce α-tubulin or acetylated α-tubulin.

The terms “inhibiting,” “eliminating,” “decreasing,” “reducing” or “preventing,” or any variation of these terms, referred to herein, includes any measurable decrease or complete inhibition to achieve a desired result.

As used herein, the term “contacting” a sample with a compound or molecule (e.g., one or more inhibitors, activators and/or inducers) refers to placing the compound in a location that will allow it to touch the sample, e.g., peripheral neurons. The contacting can be accomplished using any suitable methods. For example, contacting can be accomplished by adding the compound to a sample, e.g., contained with a tube or dish. Contacting can also be accomplished by adding the compound to a culture medium that includes the sample.

An “individual” or “subject” herein is a vertebrate, such as a human or non-human animal, for example, a mammal. Mammals include, but are not limited to, humans, primates, farm animals, sport animals, rodents, dogs, cats, and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “treating” or “treatment” (and grammatical variations thereof such as “treat”) refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state and remission or improved prognosis. By preventing progression of a disease or disorder, a treatment can prevent deterioration due to a disorder in an affected or diagnosed subject or a subject suspected of having the disorder, but also a treatment can prevent the onset of the disorder or a symptom of the disorder, e.g., peripheral neuropathy, in a subject at risk for the disorder or suspected of having the disorder. In certain embodiments, “treatment” can refer to a decrease in the severity of complications, symptoms and/or cancer or tumor growth. For example, and not by way of limitation, the decrease can be a 10%, 20%, 30%, 40%, 50%, 60%, 70%, 80%, 90%, 95%, 98% or 99% decrease in severity of complications, symptoms and/or cancer or tumor growth, for example relative to a comparable control subject not receiving the treatment. In certain embodiments, “treatment” can also mean prolonging survival as compared to expected survival if treatment is not received.

An “effective amount” (or “therapeutically effective amount”) is an amount sufficient to affect a beneficial or desired clinical result upon treatment. In certain embodiments, a therapeutically effective amount refers to an amount that is able to achieve one or more of an alleviation of neuropathic symptoms, improvement of nerve function, prolongation of survival and/or prolongation of period until relapse. For example, and not by way of limitation, a therapeutically effective amount can be an amount of a compound (e.g., inhibitor) that that minimizes, prevents, reduces and/or alleviates the symptoms of peripheral neuropathy, e.g., chemotherapy-induced peripheral neuropathy. A therapeutically effective amount can be administered to a subject in one or more doses. The therapeutically effective amount is generally determined by the physician on a case-by-case basis and is within the skill of one in the art. Several factors are typically taken into account when determining an appropriate dosage to achieve a therapeutically effective amount. These factors include age, sex and weight of the subject, the condition being treated, the severity of the condition and the form and effective concentration of the cells administered.

An “anti-cancer agent,” as used herein, can be any molecule, compound, chemical or composition that has an anti-cancer effect. Anti-cancer agents include, but are not limited to, chemotherapeutic agents, radiotherapeutic agents, cytokines, anti-angiogenic agents, apoptosis-inducing agents, anti-cancer antibodies and/or agents which promote the activity of the immune system including, but not limited to, cytokines such as but not limited to interleukin 2, interferon, anti-CTLA4 antibody, anti-PD-1 antibody and/or anti-PD-L1 antibody. In certain embodiments, the anti-cancer agent is a platinum-containing compound, e.g., oxaliplatin, cisplatin or carboplatin. Additional non-limiting examples of anti-cancer agents include paclitaxel, eribulin, thalidomide, taxanes, vinca alkaloids and bortezomib. In certain embodiments, the anti-cancer agent is a compound that affects microtubule stability, inhibits the proteasome and/or is an alkylating agent. In certain embodiments, the anti-cancer agent is a chemotherapeutic agent that has been shown to result in peripheral neuropathy, e.g., chemotherapy-induced peripheral neuropathy.

An “anti-cancer effect” refers to one or more of a reduction in aggregate cancer cell mass, a reduction in cancer cell growth rate, a reduction in cancer progression, a reduction in cancer cell proliferation, a reduction in tumor mass, a reduction in tumor volume, a reduction in tumor cell proliferation, a reduction in tumor growth rate and/or a reduction in tumor metastasis. In certain embodiments, an anti-cancer effect can refer to a complete response, a partial response, a stable disease (without progression or relapse), a response with a later relapse or progression-free survival in a patient diagnosed with cancer.

### 5.2. Deacetylase Inhibitors

In one aspect, the subject matter of the present disclosure is directed to a composition comprising inhibitors of tubulin deacetylation. In certain embodiments the tubulin is α-tubulin.

Non limiting examples of such inhibitors include compounds, molecules, chemicals, polypeptides, and proteins that inhibit the removal of the acetyl moieties from acetylated tubulin. In certain embodiments the inhibitor of tubulin deacetylation is a histone deacetylase (HDAC) inhibitor. In certain embodiments the HDAC inhibitor is a class I HDAC inhibitor. In certain embodiments the HDAC inhibitor is a class IIa HDAC inhibitor. In certain embodiments the HDAC inhibitor is a class IIb HDAC inhibitor. Non-limiting examples of HDAC inhibitors include HDAC1 inhibitors, HDAC3 inhibitors, HDAC4 inhibitors, HDAC6 inhibitors and/or HDAC10 inhibitors. In certain embodiments, the inhibitor is a pan-HDAC inhibitor. In certain embodiments, the inhibitor is a HDAC6 inhibitor. Non-limiting examples of HDAC inhibitor include, trichostatin A (TSA), Tubacin, N-hydroxy-N′-phenyl-octanediamide (also known as SAHA, Suberoylanilide hydroxamic acid, Vorinostat), and TN-301. In certain embodiments, any combination of these inhibitors can also be used. HDAC inhibitors include HDAC1 inhibitors, HDAC3 inhibitors, HDAC4 inhibitors, HDAC6 inhibitors and/or HDAC10 inhibitors. In certain embodiments, the tubulin is α-tubulin, and the HDAC is HDAC6, which is inhibited by Tubacin, TSA, N-hydroxy-N′-phenyl-octanediamide, or TN-301, or a combination thereof. Among all HDACs, HDAC6 is the primary deacetylase responsible for tubulin deacetylation.

In certain embodiments, the HDAC inhibitor is effective in inhibiting deacetylation of acetylated tubulin at a concentration between 2 nM and 1000 nM. In certain embodiments, the HDAC inhibitor is effective at a concentration from about 2 nM to about 5 nM. In certain embodiments, the HDAC inhibitor is effective at a concentration from about 2.5 nM to about 10 nM. In certain embodiments, the HDAC inhibitor is effective at a concentration from about 7.5 nM to about 15 nM. In certain embodiments, the HDAC inhibitor is effective at a concentration from about 12 nM to about 25 nM. In certain embodiments, the HDAC inhibitor is effective at a concentration from about 15 nM to about 45 nM. In certain embodiments, the HDAC inhibitor is effective at a concentration from about 30 nM to about 50 nM. In certain embodiments, the HDAC inhibitor is effective at a concentration from about 45 nM to about 75 nM. In certain embodiments, the HDAC inhibitor is effective at a concentration from about 70 nM to about 80 nM. In certain embodiments, the HDAC inhibitor is effective at a concentration from about 75 nM to about 200 nM. In certain embodiments, the HDAC inhibitor is effective at a concentration from about 150 nM to about 250 nM. In certain embodiments, the HDAC inhibitor is effective at a concentration of about 10 nM. For example, in certain embodiments, the HDAC inhibitor is TSA used at a concentration of about 10 nM.

In certain embodiments, the HDAC inhibitor is effective in inhibiting deacetylation of acetylated tubulin at a concentration between 5 μM and 100 μM. In certain embodiments, the HDAC inhibitor is effective at a concentration from about 5 μM to about 25 μM. In certain embodiments, the HDAC inhibitor is effective at a concentration from about 10 μM to about 50 μM. In certain embodiments, the HDAC inhibitor is effective at a concentration from about 30 μM to about 60 μM. In certain embodiments, the HDAC inhibitor is effective at a concentration from about 50 μM to about 75 μM. In certain embodiments, the HDAC inhibitor is effective at a concentration from about 80 μM to about 100 μM. In certain embodiments, the HDAC inhibitor is effective at a concentration of about 20 μM. For example, in certain embodiments, the HDAC inhibitor is Tubacin used at a concentration of about 20 μM.

In certain embodiments, in a CMT2A/neuropathy program, a reasonable starting point in mice with a potent, selective HDAC6 inhibitor can be selected within certain dosage ranges, for example a 3-10 mg/kg/day dosing range, which can be extractable from literature sources.

### 5.3. Acetylase Activators

In one aspect, the subject matter of the present disclosure is directed to a composition comprising activators of tubulin acetylation. In certain embodiments the tubulin is α-tubulin.

Non limiting examples of such activators include compounds, molecules, chemicals, polypeptides, and proteins that facilitate addition of acetyl moieties to tubulin (α-tubulin), for example at lysine 40. In certain embodiments the activator of tubulin acetylation is an α-tubulin acetyltransferase 1 (ATAT1) activator.

### 5.4. Nucleic Acid Compositions

In one aspect, the subject matter of the present disclosure is directed to nucleic acid composition for increasing acetylated tubulin levels. The composition comprises a nucleic acid encoding a fusion polypeptide comprising a nucleic acid sequence encoding a mitochondria targeting sequence operably linked to a nucleic acid sequence encoding a polypeptide sequence that increases levels of acetylated tubulin at sites of mitochondria contacts with microtubules.

In certain embodiments, the fusion polypeptide comprises a mitochondria targeting sequence. A non-limiting example of a mitochondria targeting sequence is outer membrane protein 25 (OMP25). In certain embodiments, the fusion polypeptide comprises a nucleic acid sequence encoding a polypeptide sequence that increases levels of acetylated tubulin. A non-limiting example of a polypeptide sequence that increases levels of acetylated tubulin is α-tubulin acetyltransferase 1 (ATAT1), which catalyzes α-tubulin acetylation for example at lysine 40. In certain embodiments, the mitochondrial targeting sequence is fused to the C-terminus of the polypeptide sequence.

In certain embodiments, the nucleic acid encoding the fusion polypeptide comprises the sequence forth in SEQ ID NO.: 3.

In certain embodiments, the nucleic acid encoding the fusion polypeptide further comprises a promoter sequence disposed upstream to the fusion polypeptide sequence. A non-limiting example of a promoter that can be used is a human synapsin I promoter (SYN1). In certain embodiments, the promoter can be selected to drive expression in neuronal cells or glial cells.

### 5.4.1. Viral Vectors

In certain embodiments, the fusion polypeptides of the disclosure are packaged in viral vectors for delivery to cells. Transduction of the cells with the viral vector enables expression of the fusion polypeptide, thereby increasing the levels of acetylated tubulin (e.g., α-tubulin) in the cells.

Non limiting examples of such viral vectors include adeno-associated virus (AAV), recombinant adenoviruses (rAV), and lentivirus (LV). In certain embodiments the viral vector is a lentiviral vector encoding the fusion polypeptide. For example, in a non-limiting embodiment, the viral vector is a lentiviral vector having a sequence set forth in SEQ ID NO.: 4, which encodes a nucleic acid encoding the ATAT1-OMP25 fusion polypeptide.

In some embodiments the fusion polypeptides of the disclosure are delivered by AAV. Non-limiting examples of AAV serotypes include AAV1, AAV2, AAV3, AAV4, AAV5, AAV6, AAV7, AAV8, AAV9, AAV10, AAV11, AAV12, AAV13. In certain embodiments, the AAV serotypes can be matched to target cell types.

### 5.5. Pharmaceutical Compositions

In another aspect, the subject matter of the present disclosure is directed to pharmaceutical compositions for increasing acetylated tubulin levels. The pharmaceutical composition comprises a nucleic acid encoding a fusion polypeptide comprising a nucleic acid sequence encoding a mitochondria targeting sequence operably linked to a nucleic acid sequence encoding a polypeptide sequence that increases levels of acetylated tubulin and a pharmaceutically acceptable carrier.

In another aspect, the subject matter of the present disclosure is directed to pharmaceutical composition comprising inhibitors of tubulin deacetylation and/or activators of tubulin acetylation and a pharmaceutically acceptable carrier. In certain aspects, the subject matter of the present disclosure is directed to pharmaceutical compositions comprising inhibitors of α-tubulin deacetylation and/or activators of α-tubulin acetylation and a pharmaceutically acceptable carrier.

In certain embodiments, pharmaceutically acceptable carriers include any carrier that does not interfere with the effectiveness of the biological activity of the active ingredients, e.g., inhibitors and activators and is not toxic to the subject to whom it is administered. Non-limiting examples of suitable pharmaceutical carriers include phosphate-buffered saline solutions, water, emulsions, such as oil/water emulsions, several types of wetting agents and sterile solutions. Additional non-limiting examples of pharmaceutically acceptable carriers can include gels, bioadsorbable matrix materials, implantation elements containing the inhibitor and/or any other suitable vehicle, delivery or dispensing means or material. Such carriers can be formulated by conventional methods and can be administered to the subject. In certain embodiments, the pharmaceutical acceptable carrier can include buffers such as phosphate, citrate, and other organic acids; antioxidants including ascorbic acid and methionine; preservatives (such as, but not limited to, octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol and m-cresol); low molecular weight (less than about 10 residues) polypeptides; proteins, such as serum albumin, gelatin or immunoglobulins; hydrophilic polymers such as polyvinylpyrrolidone; amino acids such as glycine, glutamine, asparagine, histidine, arginine or lysine; monosaccharides, disaccharides, and other carbohydrates including glucose, mannose or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). In certain embodiments, a suitable pharmaceutically acceptable carrier can include one or more of water, saline, phosphate buffered saline, dextrose, glycerol, ethanol, or combinations thereof.

In certain non-limiting embodiments, the pharmaceutical compositions can be formulated using pharmaceutically acceptable carriers well known in the art that are suitable for oral administration. Such carriers enable the pharmaceutical compositions to be formulated as tablets, pills, capsules, liquids, gels, syrups, slurries, suspensions and the like, for oral or nasal ingestion by a subject to be treated. In certain embodiments, the pharmaceutical composition can be a solid dosage form. In certain embodiments, the tablet can be an immediate release tablet. Alternatively, or additionally, the tablet can be an extended or controlled release tablet. In certain embodiments, the solid dosage can include both an immediate release portion and an extended or controlled release portion.

In certain embodiments, the pharmaceutical compositions can be formulated using pharmaceutically acceptable carriers well known in the art that are suitable for parenteral administration. The terms “parenteral administration” and “administered parenterally,” as used herein, refers to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. For example, and not by way of limitation, formulations of the present disclosure can be administered to the subject intravenously in a pharmaceutically acceptable carrier such as physiological saline. In certain embodiments, the present disclosure provides a parenteral formulation that includes a tubulin inhibitor. In certain embodiments, the present disclosure provides a parenteral formulation that includes an inhibitor of HDAC1, HDAC3, HDAC4, HDAC6 or HDAC10. In certain embodiments, a parenteral formulation of the present disclosure can include a pan-HDAC inhibitor. In certain embodiments, a parenteral formulation of the present disclosure can include an inhibitor of HDAC6. In certain embodiments, a parenteral formulation of the present disclosure can include an activator of tubulin acetylation. In certain embodiments, the present disclosure provides a parenteral formulation that includes one or more of the inhibitors and/or activators disclosed herein.

The pharmaceutical compositions can be delivered using standard methods for intracellular delivery can be used (e.g., delivery via liposome). Such methods are well known to those of ordinary skill in the art. The route of administration eventually chosen will depend upon a number of factors and can be ascertained by one skilled in the art.

In certain embodiments, the pharmaceutical compositions can include an active ingredient, e.g., HDAC inhibitor, in a therapeutically effective amount. In certain embodiments, the pharmaceutical compositions can include an inhibitor of HDAC1, HDAC3, HDAC4, HDAC6 or HDAC10, e.g., an inhibitor of HDAC6, in a therapeutically effective amount. In certain embodiments, the pharmaceutical compositions can include an activator of tubulin acetylation in a therapeutically effective amount. The therapeutically effective amount of an active ingredient can vary depending on the active ingredient, e.g., HDAC inhibitor, formulation used and the age, weight of the subject to be treated. In certain embodiments, a subject can receive a therapeutically effective amount of a HDAC inhibitor in single or multiple administrations of one or more formulations, which can depend on the dosage and frequency as required and tolerated by the subject.

In certain non-limiting embodiments, the HDAC inhibitor is trichostatin A (TSA), Tubacin, N-hydroxy-N′-phenyl-octanediamide, or TN-301 (more). In certain embodiments, the pharmaceutical composition can be a combination of the above inhibitors. For example, in certain embodiments, the tubulin is α-tubulin, the HDAC is HDAC6, and the HDAC inhibitor is TSA, Tubacin, N-hydroxy-N′-phenyl-octanediamide, or TN-301, or a combination thereof.

In certain embodiments, the pharmaceutical composition comprises the HDAC inhibitor at a concentration between 2 nM and 1000 nM. In certain embodiments, the HDAC inhibitor is at a concentration from about 2 nM to about 100 nM. In certain embodiments, the HDAC inhibitor is at a concentration from about 50 nM to about 150 nM. In certain embodiments, the HDAC inhibitor is at a concentration from about 100 nM to about 200 nM. In certain embodiments, the HDAC inhibitor is at a concentration from about 150 nM to about 250 nM. In certain embodiments, the HDAC inhibitor is at a concentration from about 200 nM to about 300 nM. In certain embodiments, the HDAC inhibitor is at a concentration from about 250 nM to about 300 nM. In certain embodiments, the HDAC inhibitor is at a concentration from about 300 nM to about 400 nM. In certain embodiments, the HDAC inhibitor is at a concentration from about 400 nM to about 500 nM. In certain embodiments, the HDAC inhibitor is at a concentration from about 500 nM to about 600 nM. In certain embodiments, the HDAC inhibitor is at a concentration from about 600 nM to about 750 nM. In certain embodiments, the HDAC inhibitor is at a concentration from about 700 nM to about 850 nM. In certain embodiments, the HDAC inhibitor is at a concentration from about 800 nM to about 1000 nM.

In certain embodiments, the pharmaceutical composition comprises the HDAC inhibitor is at a concentration between 5 μM and 1000 μM. In certain embodiments, the HDAC inhibitor is at a concentration from about 5 μM to about 25 μM. In certain embodiments, the HDAC inhibitor is at a concentration from about 10 μM to about 50 μM. In certain embodiments, the HDAC inhibitor is at a concentration from about 30 μM to about 60 μM. In certain embodiments, the HDAC inhibitor is at a concentration from about 50 μM to about 75 μM. In certain embodiments, the HDAC inhibitor is at a concentration from about 80 μM to about 100 μM. In certain embodiments, the HDAC inhibitor is at a concentration from about 50 μM to about 150 μM. In certain embodiments, the HDAC inhibitor is at a concentration from about 100 μM to about 200 μM. In certain embodiments, the HDAC inhibitor is at a concentration from about 150 μM to about 300 μM. In certain embodiments, the HDAC inhibitor is at a concentration from about 175 μM to about 300 μM. In certain embodiments, the HDAC inhibitor is at a concentration from about 250 μM to about 500 μM. In certain embodiments, the HDAC inhibitor is at a concentration from about 400 μM to about 750 μM. In certain embodiments, the HDAC inhibitor is at a concentration from about 500 μM to about 1000 μM. In certain embodiments, in a CMT2A/neuropathy program, a reasonable starting point in mice with a potent, selective HDAC6 inhibitor can be selected within certain dosage ranges, for example a 3-10 mg/kg/day dosing range, which can be extractable from literature sources.

In another aspect, the subject matter of the present disclosure is directed to use of a compound that increases tubulin acetylation, in the manufacture of a medicament for the treatment of peripheral neuropathy in a subject.

In certain embodiments, the compound is an inhibitor of tubulin deacetylation. In certain embodiments, the compound is an activator of tubulin acetylation. In certain embodiments, the medicament comprises at least one HDAC inhibitor, in a therapeutically effective amount. In certain embodiments, the medicament comprises a pan-HDAC inhibitor, in a therapeutically effective amount. In certain embodiments, the medicament comprises an inhibitor of HDAC1, HDAC3, HDAC4, HDAC6 or HDAC10, in a therapeutically effective amount. In certain embodiments, the medicament comprises an inhibitor of HDAC6, in a therapeutically effective amount. In certain embodiments, the medicament comprises an activator of tubulin acetylation in a therapeutically effective amount. The therapeutically effective amount of the HDAC inhibitor, tubulin acetylation activator can vary depending on the formulation used and the age, weight of the subject to be treated. In certain embodiments, the medicament is adapted for use in single or multiple administrations of one or more formulations, which can depend on the dosage and frequency as required and tolerated by the subject. In certain embodiments the tubulin acetylation activator is an α-tubulin acetyltransferase 1 (ATAT1) activator. In certain embodiments, a combination of a tubulin deacetylation inhibitor and a tubulin acetylation activator can be used, whereby the levels of acetylated tubulin (e.g., α-tubulin) is promoted.

In certain non-limiting embodiments, the HDAC inhibitor is trichostatin A (TSA), Tubacin, N-hydroxy-N′-phenyl-octanediamide, or TN-301. In certain embodiments, the pharmaceutical composition can be a combination of the above inhibitors. For example, in certain embodiments, the tubulin is α-tubulin, the HDAC is HDAC6, and the HDAC inhibitor is TSA, Tubacin, N-hydroxy-N′-phenyl-octanediamide, or TN-301, or a combination thereof. In particular embodiments, the HDAC inhibitors in its various embodiments are as described in Sections 5.2 and 5.5.

### 5.6. Methods

### 5.6.1. Methods of Use

In another aspect, the subject matter of the present disclosure is directed to methods for determining whether a subject is at risk of developing peripheral neuropathy, comprising obtaining a test sample from a subject who has not been diagnosed with suffering from peripheral neuropathy; and determining in the sample, a level of acetylated tubulin; wherein a lower level of acetylated tubulin in the test sample compared to a control level indicates that the subject is at an increased risk of developing peripheral neuropathy.

In another aspect, the subject matter of the present disclosure is directed to methods for determining whether a subject has peripheral neuropathy, comprising obtaining a test sample from the subject; and determining in the sample, a level of acetylated tubulin; wherein a lower level of acetylated tubulin compared to a control level indicates that the subject has peripheral neuropathy in need of treatment.

In certain non-limiting embodiments, the sample is a tissue sample, a blood sample, or a biopsy sample. In certain embodiments, the sample is a biopsy sample of a peripheral neuron. In certain embodiments, the sample is a dermal biopsy. In certain embodiments, the sample comprises one or more peripheral neuronal cells or PBMCs. Non-limiting examples of peripheral neuronal cells include sensory neuronal cells and motor neuronal cells.

In certain embodiments, determining the level of acetylated tubulin can by performed by using quantitative assays. In certain non-limiting embodiments, the assay quantifies acetylated tubulin protein levels by customized ELISA, immunofluorescence, western blotting, or mass spectrometry. A combination of assays can also be used. Reagents for immunofluorescence and western blotting can include detectable labels that are associated with, or linked to a binding partner, such as for example, an antibody (or antibody fragments e.g., Fab, Fab′, F(ab′)₂, scFv, (scFv)₂, single-chain antibody, VHH antibody and minibody), a protein, or a small molecule that selectively binds acetylated tubulin over non-acetylated tubulin. Such detectable labels include, for example, chemiluminescent or fluorescent molecules (e.g., rhodamine, fluorescein, green fluorescent protein, luciferase, Cy3, Cy5, or ROX), radiolabels (e.g., ³H, ³⁵S, ³²P, ¹⁴C and ¹³¹I) or enzymes (e.g., alkaline phosphatase, horseradish peroxidase). Alternatively, a detectable moiety can be included in a secondary antibody or antibody fragment, which selectively binds to the first antibody or antibody fragment, where the first antibody or antibody fragment specifically recognizes acetylated tubulin.

In certain embodiments, the level of acetylated tubulin is compared with a control level of acetylated tubulin. In certain embodiments, the control level of acetylated tubulin is obtained from a reference standard. As a non-limiting example, the reference standard can be established using samples obtained from a population of healthy subjects. In certain embodiments, the control level of acetylated tubulin is obtained from a control sample (e.g., from a healthy subject) that is analyzed concurrently with the test sample.

In some aspects, the method further comprises treating the subject identified as at risk of developing peripheral neuropathy or the subject having peripheral neuropathy. In certain embodiments, treating comprises administering to the subject, a therapeutically effective amount of a compound that increases tubulin acetylation.

### 5.6.2. Inhibitors and Activators for Promoting Levels of Acetylated Tubulin

In certain embodiments, treatment comprises administering to the subject, a therapeutically effective amount of a compound that increases levels of acetylated tubulin. In certain embodiments, the compound is an inhibitor of tubulin deacetylation. In certain embodiments, the compound is an activator of tubulin acetylation. Non limiting examples of tubulin deacetylation inhibitors and tubulin acetylation activators include compounds, molecules, chemicals, polypeptides, and proteins that inhibit the removal of the acetyl moieties from acetylated tubulin (inhibitors) or facilitate addition of acetyl moieties to tubulin (activators). In certain embodiments the tubulin is α-tubulin.

In certain embodiments the inhibitor of tubulin deacetylation is a histone deacetylase (HDAC) inhibitor. In certain embodiments the HDAC inhibitor is a class I HDAC inhibitor. In certain embodiments the HDAC inhibitor is a class IIa HDAC inhibitor. In certain embodiments the HDAC inhibitor is a class IIb HDAC inhibitor. Non-limiting examples of HDAC inhibitors include HDAC1 inhibitors, HDAC3 inhibitors, HDAC4 inhibitors, HDAC6 inhibitors and/or HDAC10 inhibitors. In certain embodiments, the inhibitor is a pan-HDAC inhibitor. In certain embodiments, the inhibitor is a HDAC6 inhibitor. Non-limiting examples of HDAC inhibitor include, trichostatin A (TSA), Tubacin, N-hydroxy-N′-phenyl-octanediamide (also known as SAHA, Suberoylanilide hydroxamic acid, Vorinostat), and TN-301. In certain embodiments, any combination of these inhibitors can also be used. In certain embodiments, the tubulin is α-tubulin, and the HDAC is HDAC6, which is inhibited by Tubacin, TSA, N-hydroxy-N′-phenyl-octanediamide, or TN-301, or a combination thereof.

In certain embodiments, the HDAC inhibitor is effective in inhibiting deacetylation of acetylated tubulin at a concentration between 2 nM and 1000 nM. In certain embodiments, the HDAC inhibitor is effective at a concentration from about 2 nM to about 5 nM. In certain embodiments, the HDAC inhibitor is effective at a concentration from about 2.5 nM to about 10 nM. In certain embodiments, the HDAC inhibitor is effective at a concentration from about 7.5 nM to about 15 nM. In certain embodiments, the HDAC inhibitor is effective at a concentration from about 12 nM to about 25 nM. In certain embodiments, the HDAC inhibitor is effective at a concentration from about 15 nM to about 45 nM. In certain embodiments, the HDAC inhibitor is effective at a concentration from about 30 nM to about 50 nM. In certain embodiments, the HDAC inhibitor is effective at a concentration from about 45 nM to about 75 nM. In certain embodiments, the HDAC inhibitor is effective at a concentration from about 70 nM to about 80 nM. In certain embodiments, the HDAC inhibitor is effective at a concentration from about 75 nM to about 200 nM. In certain embodiments, the HDAC inhibitor is effective at a concentration from about 150 nM to about 250 nM. In certain embodiments, the HDAC inhibitor is effective at a concentration of about 10 nM. For example, in certain embodiments, the HDAC inhibitor is TSA used at a concentration of about 10 nM.

In certain embodiments, the HDAC inhibitor is effective in inhibiting deacetylation of acetylated tubulin at a concentration between 5 μM and 100 μM. In certain embodiments, the HDAC inhibitor is effective at a concentration from about 5 μM to about 25 μM. In certain embodiments, the HDAC inhibitor is effective at a concentration from about 10 μM to about 50 μM. In certain embodiments, the HDAC inhibitor is effective at a concentration from about 30 μM to about 60 μM. In certain embodiments, the HDAC inhibitor is effective at a concentration from about 50 μM to about 75 μM. In certain embodiments, the HDAC inhibitor is effective at a concentration from about 80 μM to about 100 μM. In certain embodiments, the HDAC inhibitor is effective at a concentration of about 20 μM. For example, in certain embodiments, the HDAC inhibitor is Tubacin used at a concentration of about 20 μM.

In certain embodiments, the subject matter of the present disclosure is directed to a composition comprising activators of tubulin acetylation. Non limiting examples of such activators include compounds, molecules, chemicals, polypeptides, and proteins that facilitate addition of acetyl moieties to lysine 40 of tubulin (α-tubulin). In certain embodiments the activator of tubulin acetylation is an α-tubulin acetyltransferase 1 (ATAT1) activator.

In another aspect, the subject matter of the present disclosure is directed to methods for identifying a compound as effective for treatment of peripheral neuropathy, comprising obtaining a sample; determining an original level of acetylated tubulin in the sample (first determining); contacting the sample with a compound that increases tubulin acetylation; and detecting in the sample an updated level of acetylated tubulin (second determining); wherein, a higher level of acetylated tubulin compared to the original level indicates that the compound is effective for treatment of peripheral neuropathy.

In certain non-limiting embodiments, the sample is a tissue sample, a blood sample PBMCs, or a biopsy sample. In certain embodiments, the sample is a biopsy sample of a peripheral neuron. In certain embodiments, the sample is a dermal biopsy. In certain embodiments, the sample comprises one or more peripheral neuronal cells. Non-limiting examples of peripheral neuronal cells include sensory neuronal cells and motor neuronal cells.

In certain embodiments, determining a level of acetylated tubulin can by performed by using quantitative assays. In certain non-limiting embodiments, the assay quantifies acetylated tubulin protein levels by customized ELISA, immunofluorescence, western blotting, or mass spectrometry. A combination of assays can also be used.

In certain embodiments, the acetylated tubulin is acetylated α-tubulin.

In certain embodiments, the compound increases levels of acetylated tubulin. In certain embodiments, the compound is an inhibitor of tubulin deacetylation. In certain embodiments, the compound is an activator of tubulin acetylation. Non limiting examples of tubulin deacetylation inhibitors and tubulin acetylation activators include compounds, molecules, chemicals, polypeptides, and proteins that inhibit the removal of the acetyl moieties from acetylated tubulin (inhibitors) or facilitate addition of acetyl moieties to lysine 40 of alpha-tubulin (activators). In certain embodiments the tubulin is α-tubulin.

In certain embodiments the inhibitor of tubulin deacetylation is a histone deacetylase (HDAC) inhibitor. In certain embodiments the HDAC inhibitor is a class I HDAC inhibitor. In certain embodiments the HDAC inhibitor is a class IIa HDAC inhibitor. In certain embodiments the HDAC inhibitor is a class IIb HDAC inhibitor. Non-limiting examples of HDAC inhibitors include HDAC1 inhibitors, HDAC3 inhibitors, HDAC4 inhibitors, HDAC6 inhibitors and/or HDAC10 inhibitors. In certain embodiments, the inhibitor is a pan-HDAC inhibitor. In certain embodiments, the inhibitor is a HDAC6 inhibitor. Non-limiting examples of HDAC inhibitor include, trichostatin A (TSA), Tubacin, N-hydroxy-N′-phenyl-octanediamide (also known as SAHA, Suberoylanilide hydroxamic acid, Vorinostat), and TN-301. In certain embodiments, any combination of these inhibitors can also be used. In certain embodiments, the tubulin is α-tubulin, and the HDAC is HDAC6, which is inhibited by Tubacin, TSA, N-hydroxy-N′-phenyl-octanediamide, or TN-301, or a combination thereof. In particular embodiments, the HDAC inhibitors in its various embodiments are as described in Sections 5.2 and 5.5.

### 5.6.3. Methods of Treatment

In another aspect, the subject matter of the present disclosure is directed to methods for treating a subject suffering from a disease associated with peripheral neuropathy, comprising administering to the subject a composition comprising a therapeutically effective amount of a compound that increases tubulin acetylation.

In non-limiting embodiments, the disease associated with peripheral neuropathy is diabetes, hypothyroidism, kidney disease, liver disease, cancer, Charcot-Marie-Tooth (CMT) disease, Guillain-Barré syndrome, autoimmune disease, rheumatoid arthritis, Lyme disease and lupus. In certain embodiments, the disease associated with peripheral neuropathy is CMT disease. In certain embodiments, the disease associated with peripheral neuropathy is diabetes or diabetic peripheral neuropathy. In certain embodiments, the diabetic peripheral neuropathy is chemotherapy-induced peripheral neuropathy (CIPN), including vincristine-induced peripheral neuropathy.

In certain embodiments the inhibitor of tubulin deacetylation is a histone deacetylase (HDAC) inhibitor. In certain embodiments the HDAC inhibitor is a class I HDAC inhibitor. In certain embodiments the HDAC inhibitor is a class IIa HDAC inhibitor. In certain embodiments the HDAC inhibitor is a class IIb HDAC inhibitor. Non-limiting examples of HDAC inhibitors include HDAC1 inhibitors, HDAC3 inhibitors, HDAC4 inhibitors, HDAC6 inhibitors and/or HDAC10 inhibitors. In certain embodiments, the inhibitor is a pan-HDAC inhibitor. In certain embodiments, the inhibitor is a HDAC6 inhibitor. Non-limiting examples of HDAC inhibitor include, trichostatin A (TSA), Tubacin, N-hydroxy-N′-phenyl-octanediamide (also known as SAHA, Suberoylanilide hydroxamic acid, Vorinostat), and TN-301. In certain embodiments, any combination of these inhibitors can also be used. In certain embodiments, the tubulin is α-tubulin, and the HDAC is HDAC6, which is inhibited by Tubacin, TSA, N-hydroxy-N′-phenyl-octanediamide, or TN-301, or a combination thereof. HDAC inhibitors include HDAC1 inhibitors, HDAC3 inhibitors, HDAC4 inhibitors, HDAC6 inhibitors and/or HDAC10 inhibitors. In certain embodiments, the HDAC inhibitor is effective in inhibiting deacetylation of acetylated tubulin at a concentration between 2 nM and 1000 nM. In certain embodiments, the HDAC inhibitor is effective at a concentration from about 2 nM to about 5 nM. In certain embodiments, the HDAC inhibitor is effective at a concentration from about 2.5 nM to about 10 nM. In certain embodiments, the HDAC inhibitor is effective at a concentration from about 7.5 nM to about 15 nM. In certain embodiments, the HDAC inhibitor is effective at a concentration from about 12 nM to about 25 nM. In certain embodiments, the HDAC inhibitor is effective at a concentration from about 15 nM to about 45 nM. In certain embodiments, the HDAC inhibitor is effective at a concentration from about 30 nM to about 50 nM. In certain embodiments, the HDAC inhibitor is effective at a concentration from about 45 nM to about 75 nM. In certain embodiments, the HDAC inhibitor is effective at a concentration from about 70 nM to about 80 nM. In certain embodiments, the HDAC inhibitor is effective at a concentration from about 75 nM to about 200 nM. In certain embodiments, the HDAC inhibitor is effective at a concentration from about 150 nM to about 250 nM. In certain embodiments, the HDAC inhibitor is effective at a concentration of about 10 nM. For example, in certain embodiments, the HDAC inhibitor is TSA used at a concentration of about 10 nM.

In certain embodiments, the HDAC inhibitor is effective in inhibiting deacetylation of acetylated tubulin at a concentration between 5 μM and 100 μM. In certain embodiments, the HDAC inhibitor is effective at a concentration from about 5 μM to about 25 μM. In certain embodiments, the HDAC inhibitor is effective at a concentration from about 10 μM to about 50 μM. In certain embodiments, the HDAC inhibitor is effective at a concentration from about 30 μM to about 60 μM. In certain embodiments, the HDAC inhibitor is effective at a concentration from about 50 μM to about 75 μM. In certain embodiments, the HDAC inhibitor is effective at a concentration from about 80 μM to about 100 μM. In certain embodiments, the HDAC inhibitor is effective at a concentration of about 20 μM. For example, in certain embodiments, the HDAC inhibitor is Tubacin used at a concentration of about 20 μM.

In certain embodiments, the subject matter of the present disclosure is directed to a composition comprising activators of tubulin acetylation. Non limiting examples of such activators include compounds, molecules, chemicals, polypeptides, and proteins that facilitate addition of acetyl moieties to tubulin (α-tubulin). In certain embodiments the activator of tubulin acetylation is an α-tubulin acetyltransferase 1 (ATAT1) activator.

In another aspect, the subject matter of the present disclosure is directed to methods for treating a subject comprising, determining a level of acetylated tubulin in a sample obtained from a subject; identifying the subject as suffering from a peripheral neuropathy when the level of acetylated tubulin in the sample from the subject is lower compared to a control level; and administering to the subject identified as suffering from a peripheral neuropathy, a composition comprising an inhibitor of tubulin deacetylation and/or an activator of tubulin acetylation.

In certain non-limiting embodiments, the sample is a tissue sample, a blood sample, or a biopsy sample. In certain embodiments, the sample is a biopsy sample of a peripheral neuron. In certain embodiments, the sample is a dermal biopsy. In certain embodiments, the sample comprises one or more peripheral neuronal cells. Non-limiting examples of peripheral neuronal cells include sensory neuronal cells and motor neuronal cells.

In certain embodiments, determining the level of acetylated tubulin can by performed by using quantitative assays. In certain non-limiting embodiments, the assay quantifies acetylated tubulin protein levels by immunofluorescence, western blotting, or mass spectrometry. A combination of assays can also be used. Reagents for immunofluorescence and western blotting can include detectable labels that are associated with, or linked to a binding partner, such as for example, an antibody (or antibody fragments e.g., Fab, Fab′, F(ab′)₂, scFv, (scFv)₂, single-chain antibody, VHH antibody and minibody), a protein, or a small molecule that selectively binds acetylated tubulin over non-acetylated tubulin. Such detectable labels include, for example, chemiluminescent or fluorescent molecules (e.g., rhodamine, fluorescein, green fluorescent protein, luciferase, Cy3, Cy5, or ROX), radiolabels (e.g., ³H, ³⁵S, ³²P, ¹⁴C and ¹³¹I) or enzymes (e.g., alkaline phosphatase, horseradish peroxidase). Alternatively, a detectable moiety can be included in a secondary antibody or antibody fragment, which selectively binds to the first antibody or antibody fragment, where the first antibody or antibody fragment specifically recognizes acetylated tubulin.

In certain embodiments, the level of acetylated tubulin is compared with a control level of acetylated tubulin. In certain embodiments, the control level of acetylated tubulin is obtained from a reference standard. As a non-limiting example, the reference standard can be established using samples obtained from a population of healthy subjects. In certain embodiments, the control level of acetylated tubulin is obtained from a control sample (e.g., from a healthy subject) that is analyzed concurrently with the test sample.

In certain embodiments, the inhibitor of tubulin deacetylation is a deacetylation inhibitor. Non limiting examples of tubulin deacetylation inhibitors and tubulin acetylation activators include compounds, molecules, chemicals, polypeptides, and proteins that inhibit the removal of the acetyl moieties from acetylated tubulin (inhibitors) or facilitate addition of acetyl moieties to tubulin (activators). For example, in certain embodiments the activator of tubulin acetylation is an α-tubulin acetyltransferase 1 (ATAT1) activator. In certain embodiments the tubulin is α-tubulin.

In certain embodiments the inhibitor of tubulin deacetylation is a histone deacetylase (HDAC) inhibitor. In certain embodiments the HDAC inhibitor is a class I HDAC inhibitor. In certain embodiments the HDAC inhibitor is a class IIa HDAC inhibitor. In certain embodiments the HDAC inhibitor is a class IIb HDAC inhibitor. Non-limiting examples of HDAC inhibitors include HDAC1 inhibitors, HDAC3 inhibitors, HDAC4 inhibitors, HDAC6 inhibitors and/or HDAC10 inhibitors. In certain embodiments, the inhibitor is a pan-HDAC inhibitor. In certain embodiments, the inhibitor is a HDAC6 inhibitor. Non-limiting examples of HDAC inhibitor include, trichostatin A (TSA), Tubacin, N-hydroxy-N′-phenyl-octanediamide (also known as SAHA, Suberoylanilide hydroxamic acid, Vorinostat), and TN-301. In certain embodiments, any combination of these inhibitors can also be used. In certain embodiments, the tubulin is α-tubulin, and the HDAC is HDAC6, which is inhibited by Tubacin, TSA, N-hydroxy-N′-phenyl-octanediamide, or TN-301, or a combination thereof.

In certain embodiments, the HDAC inhibitor is effective in inhibiting deacetylation of acetylated tubulin at a concentration between 2 nM and 1000 nM. In certain embodiments, the HDAC inhibitor is effective at a concentration from about 2 nM to about 5 nM. In certain embodiments, the HDAC inhibitor is effective at a concentration from about 2.5 nM to about 10 nM. In certain embodiments, the HDAC inhibitor is effective at a concentration from about 7.5 nM to about 15 nM. In certain embodiments, the HDAC inhibitor is effective at a concentration from about 12 nM to about 25 nM. In certain embodiments, the HDAC inhibitor is effective at a concentration from about 15 nM to about 45 nM. In certain embodiments, the HDAC inhibitor is effective at a concentration from about 30 nM to about 50 nM. In certain embodiments, the HDAC inhibitor is effective at a concentration from about 45 nM to about 75 nM. In certain embodiments, the HDAC inhibitor is effective at a concentration from about 70 nM to about 80 nM. In certain embodiments, the HDAC inhibitor is effective at a concentration from about 75 nM to about 200 nM. In certain embodiments, the HDAC inhibitor is effective at a concentration from about 150 nM to about 250 nM. In certain embodiments, the HDAC inhibitor is effective at a concentration of about 10 nM. For example, in certain embodiments, the HDAC inhibitor is TSA used at a concentration of about 10 nM.

In certain embodiments, the HDAC inhibitor is effective in inhibiting deacetylation of acetylated tubulin at a concentration between 5 μM and 100 μM. In certain embodiments, the HDAC inhibitor is effective at a concentration from about 5 μM to about 25 μM. In certain embodiments, the HDAC inhibitor is effective at a concentration from about 10 μM to about 50 μM. In certain embodiments, the HDAC inhibitor is effective at a concentration from about 30 μM to about 60 μM. In certain embodiments, the HDAC inhibitor is effective at a concentration from about 50 μM to about 75 μM. In certain embodiments, the HDAC inhibitor is effective at a concentration from about 80 μM to about 100 μM. In certain embodiments, the HDAC inhibitor is effective at a concentration of about 20 μM. For example, in certain embodiments, the HDAC inhibitor is Tubacin used at a concentration of about 20 μM.

In another aspect, the subject matter of the present disclosure is directed to methods for treating a subject suffering from a disease associated with peripheral neuropathy comprising administering to the subject, a composition comprising a nucleic acid encoding a fusion polypeptide comprising a nucleic acid sequence encoding a mitochondria targeting sequence operably linked to a nucleic acid sequence encoding a polypeptide sequence that increases levels of acetylated tubulin.

In another aspect, the subject matter of the present disclosure is directed to methods for treating a subject comprising, determining a level of acetylated tubulin in a sample obtained from a subject; identifying the subject as suffering from a peripheral neuropathy when the level of acetylated tubulin in the sample from the subject is lower compared to a control level; and administering to the subject identified as suffering from a peripheral neuropathy, a composition comprising a nucleic acid encoding a fusion polypeptide comprising a nucleic acid sequence encoding a mitochondria targeting sequence operably linked to a nucleic acid sequence encoding a polypeptide sequence that increases levels of acetylated tubulin.

In another aspect, the subject matter of the present disclosure is directed to methods for treating a subject suffering from a disease associated with neuropathy comprising, administering to the subject, a composition comprising a nucleic acid encoding a fusion polypeptide comprising a nucleic acid sequence encoding outer membrane protein 25 (OMP25); a nucleic acid sequence encoding α-tubulin acetyltransferase 1 (ATAT1) operably linked to OMP25; and a human synapsin I promoter (SYN1) sequence disposed upstream to the nucleic acid encoding the fusion polypeptide sequence; wherein the OMP25 peptide is fused to the C-terminus of ATAT1; and wherein the nucleic acid encoding the fusion polypeptide comprises the sequence set forth in SEQ ID NO.: 3.

In certain embodiments, treating the subject comprises administering a pharmaceutical composition that further comprises a pharmaceutically acceptable carrier. In particular embodiments, the pharmaceutical composition in its various embodiments are as described in Section 5.5.

In certain embodiments, the neuropathy is associated with diabetes, hypothyroidism, kidney disease, liver disease, cancer, Charcot-Marie-Tooth (CMT) disease, chemotherapy-induced peripheral neuropathy (CIPN), Guillain-Barré syndrome, autoimmune disease, rheumatoid arthritis, Lyme disease and lupus. In certain embodiments, the neuropathy is a peripheral neuropathy.

In certain embodiments, the composition (e.g., pharmaceutical composition) is administered to a peripheral nerve in the subject. In certain embodiments, the composition (e.g., pharmaceutical composition) is administered to a neuronal cell in the subject. In certain embodiments, the composition (e.g., pharmaceutical composition) is administered to a peripheral neuronal cell in the subject. In certain embodiments, the composition (e.g., pharmaceutical composition) is administered to a sensory neuron in the subject.

In certain embodiments, the fusion polypeptide comprises a mitochondria targeting sequence. A non-limiting example of a mitochondria targeting sequence is outer membrane protein 25 (OMP25). In certain embodiments, the fusion polypeptide comprises a nucleic acid sequence encoding a polypeptide sequence that increases levels of acetylated tubulin. A non-limiting example of a polypeptide sequence that increases levels of acetylated tubulin is α-tubulin acetyltransferase 1 (ATAT1), which catalyzes α-tubulin acetylation. In certain embodiments, the mitochondrial targeting sequence is fused to the C-terminus of the polypeptide sequence.

In certain embodiments, the nucleic acid encoding the fusion polypeptide comprises the sequence forth in SEQ ID NO.: 3.

In certain embodiments, the nucleic acid encoding the fusion polypeptide further comprises a promoter sequence disposed upstream to the fusion polypeptide sequence. A non-limiting example of a promoter that can be used is a human synapsin I promoter (SYN1), which preferentially directs expression in neuronal cells and may be suitable for axonal forms of Charcot-Marie-Tooth (CMT) disease or other neuropathies involving neuronal dysfunction.

In certain embodiments, the promoter can be a Schwann cell-specific promoter suitable for targeting demyelinating forms of CMT or peripheral neuropathy. Exemplary promoters include myelin-associated promoters, such as a myelin protein zero (MPZ) promoter, which has demonstrated in vivo activity in Schwann cells and may be used to direct gene expression to myelinating glial cells of the peripheral nervous system.

In certain embodiments, the fusion polypeptides of the disclosure are packaged in viral vectors for delivery to cells. Transduction of the cells with the viral vector enables expression of the fusion polypeptide, thereby increasing the levels of acetylated tubulin (e.g., α-tubulin) in the cell. Non limiting examples of such viral vectors include adeno-associated virus (AAV), recombinant adenoviruses (rAV), and lentivirus (LV). In certain embodiments the viral vector is a lentiviral vector encoding the fusion polypeptide. For example, in a non-limiting embodiment, the viral vector is a lentiviral vector having a sequence set forth in SEQ ID NO.: 4, which encodes a nucleic acid encoding the ATAT1-OMP25 fusion polypeptide.

### 5.7. Kits

In another aspect, the subject matter of the present disclosure is directed to a kit for identifying a compound effective for the treatment of peripheral neuropathy comprising, a specific binding partner for acetylated tubulin, a sample comprising neuronal cells, and instructions for identifying the compound. In certain embodiments, identifying the compound comprises obtaining a sample, determining an original level of acetylated tubulin in the sample contacting the sample with a compound that increases levels of tubulin acetylation, and detecting in the sample an updated level of acetylated tubulin, where a higher level of acetylated tubulin compared to the original level indicates that the compound is effective for treatment of peripheral neuropathy.

In certain embodiments, the specific binding partner is an antibody that binds for acetylated tubulin (e.g., for acetylated α-tubulin). In certain embodiments, the specific binding partner for acetylated tubulin is an antibody fragment. In certain non-limiting embodiments, the antibody fragment is selected from the group consisting of: a Fab, a Fab′, an F(ab′)₂, an scFv, an (scFv)₂, a single-chain antibody, a VHH antibody and a minibody.

In certain embodiments, the neuronal cell is a peripheral neuronal cell. In certain embodiments, the neuronal cell is a sensory neuronal cell, or a motor neuronal cell. In certain embodiments, the peripheral neuronal cell is a sensory neuronal cell, or a motor neuronal cell.

In certain embodiments, determining the level of acetylated tubulin can by performed by using quantitative assays. In certain non-limiting embodiments, the assay quantifies acetylated tubulin protein levels by immunofluorescence, western blotting, or mass spectrometry. A combination of assays can also be used. Reagents for immunofluorescence and western blotting can include detectable labels that are associated with, or linked to a binding partner, such as for example, an antibody (or antibody fragments e.g., Fab, Fab′, F(ab′)₂, scFv, (scFv)₂, single-chain antibody, VHH antibody and minibody), a protein, or a small molecule that selectively binds acetylated tubulin over non-acetylated tubulin. Such detectable labels include, for example, chemiluminescent or fluorescent molecules (e.g., rhodamine, fluorescein, green fluorescent protein, luciferase, Cy3, Cy5, or ROX), radiolabels (e.g., ³H, ³⁵S, ³²P, ¹⁴C and ¹³¹I) or enzymes (e.g., alkaline phosphatase, horseradish peroxidase). Alternatively, a detectable moiety can be included in a secondary antibody or antibody fragment, which selectively binds to the first antibody or antibody fragment, where the first antibody or antibody fragment specifically recognizes acetylated tubulin.

In certain embodiments, the inhibitor of tubulin deacetylation is a deacetylation inhibitor. Non limiting examples of tubulin deacetylation inhibitors and tubulin acetylation activators include compounds, molecules, chemicals, polypeptides, and proteins that inhibit the removal of the acetyl moieties from acetylated tubulin (inhibitors) or facilitate addition of acetyl moieties to tubulin (activators). For example, in certain embodiments the activator of tubulin acetylation is an α-tubulin acetyltransferase 1 (ATAT1) activator. In certain embodiments the tubulin is α-tubulin.

In certain embodiments, the compound that increases levels of acetylated tubulin is a tubulin deacetylase inhibitor. In certain embodiments, the tubulin deacetylase inhibitor is a HDAC inhibitor. Non-limiting examples of HDAC inhibitors include HDAC1 inhibitors, HDAC3 inhibitors, HDAC4 inhibitors, HDAC6 inhibitors and/or HDAC10 inhibitors. In certain embodiments, the inhibitor is a pan-HDAC inhibitor. In certain embodiments, the inhibitor is a HDAC6 inhibitor. Non-limiting examples of HDAC inhibitor include, trichostatin A (TSA), Tubacin, N-hydroxy-N′-phenyl-octanediamide (also known as SAHA, Suberoylanilide hydroxamic acid, Vorinostat), and TN-301. In certain embodiments, any combination of these inhibitors can also be used. In certain embodiments, the tubulin is α-tubulin, and the HDAC is HDAC6, which is inhibited by Tubacin, TSA, N-hydroxy-N′-phenyl-octanediamide, or TN-301, or a combination thereof. In certain embodiments, the activator of tubulin acetylation include compounds, molecules, chemicals, polypeptides, and proteins that facilitate addition of acetyl moieties to tubulin (α-tubulin). In certain embodiments, the compound that increases levels of acetylated tubulin is an α-tubulin acetyltransferase 1 (ATAT1) activator.

### 6. EXAMPLES

### 6.1. Example 1. Materials and Methods

A list of materials employed is shown in Table 1.

TABLE 1
Materials and Resource Identifier

| | | |
|---|---|---|
| Reagent/Resource | Source | Catalog#/Identifier |

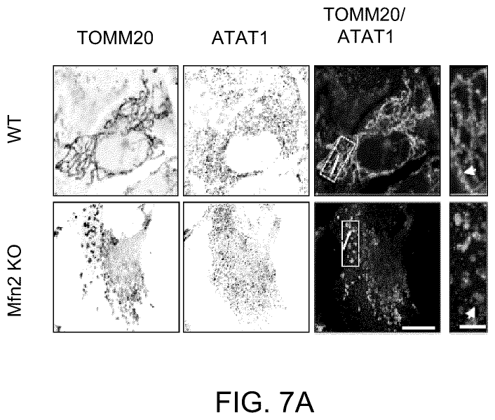

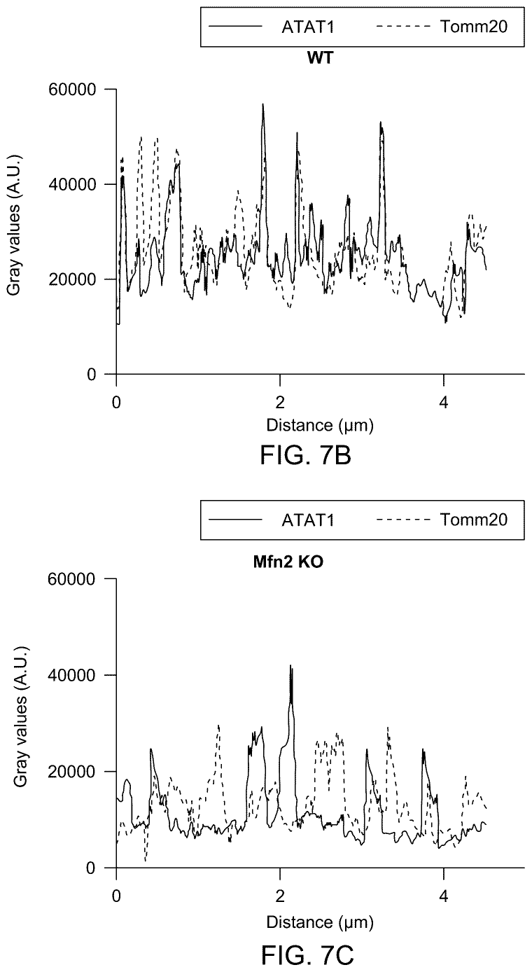

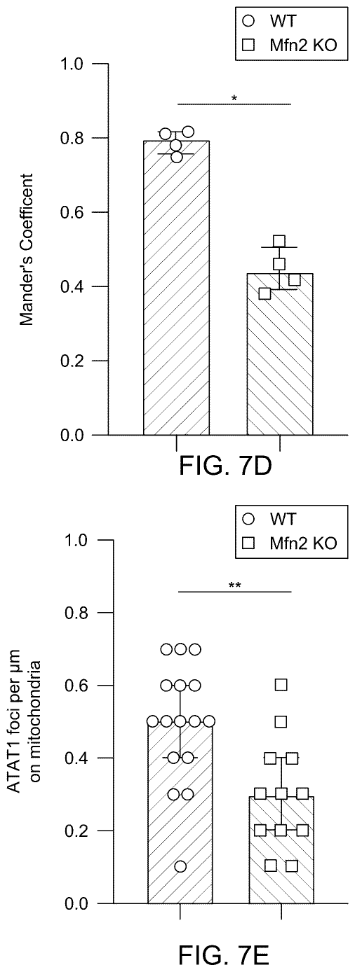

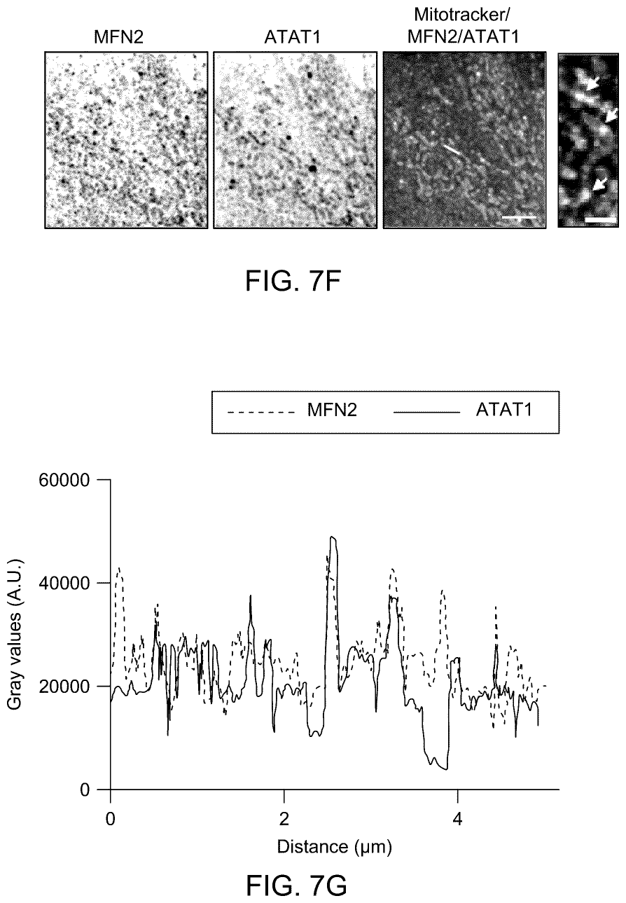

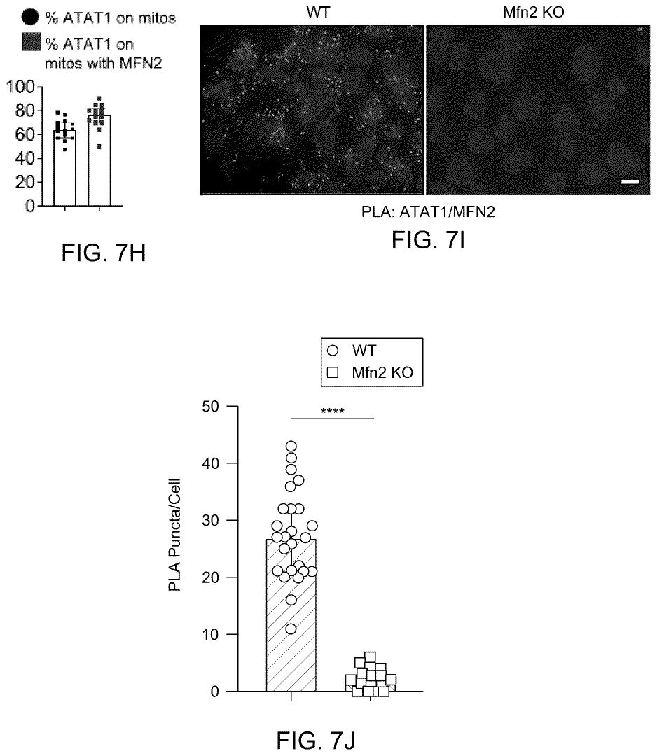

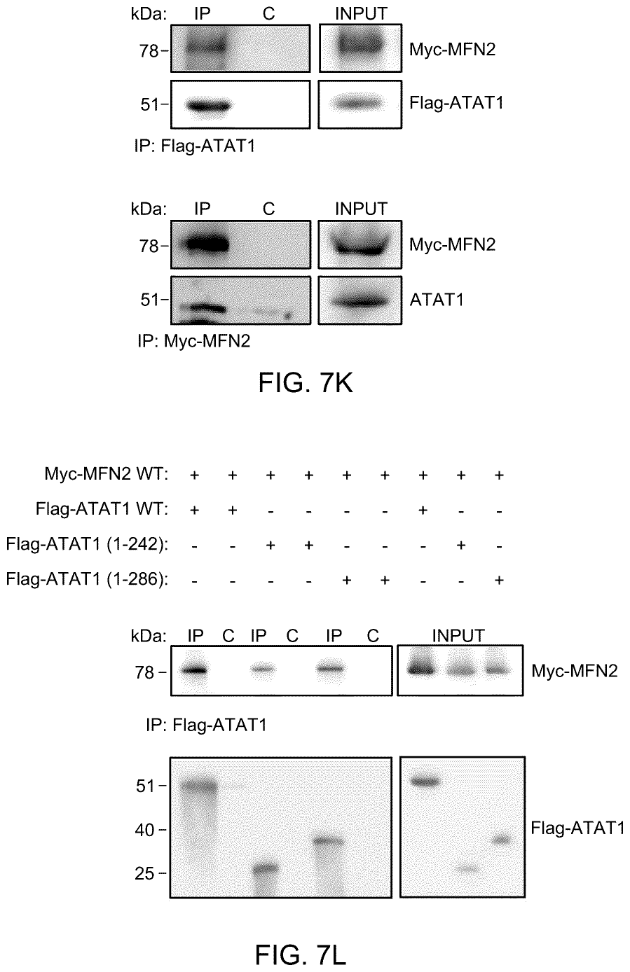

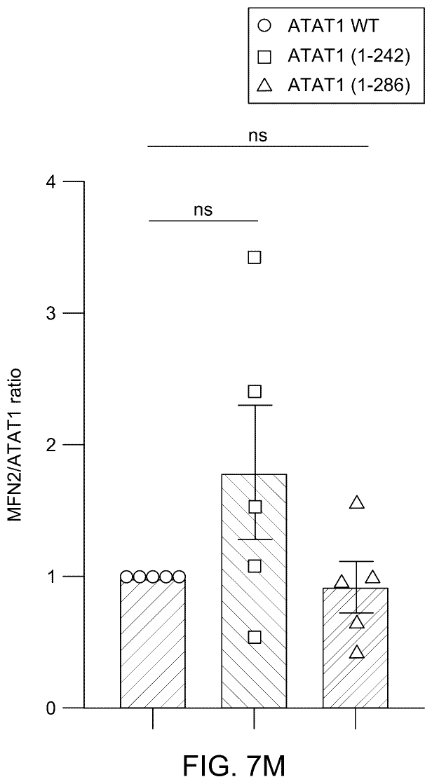

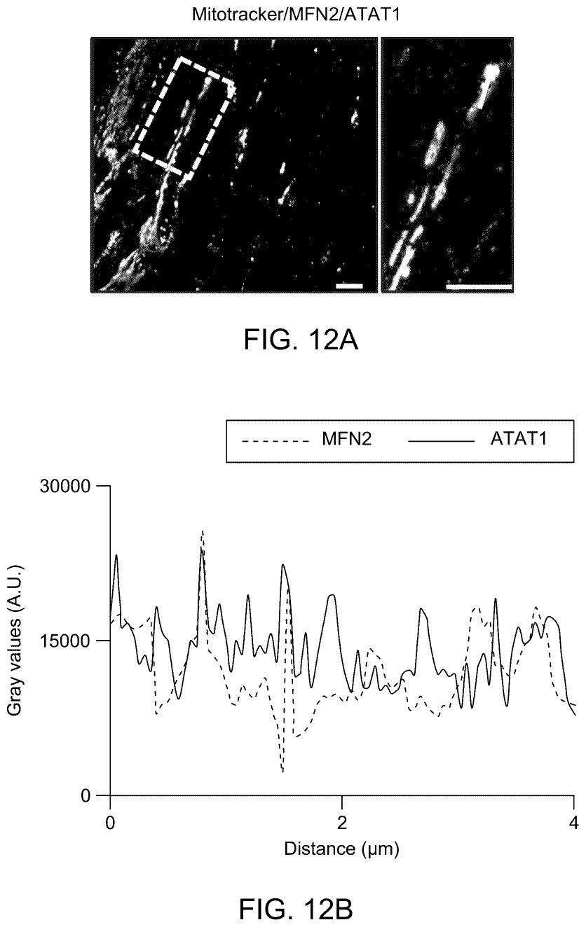

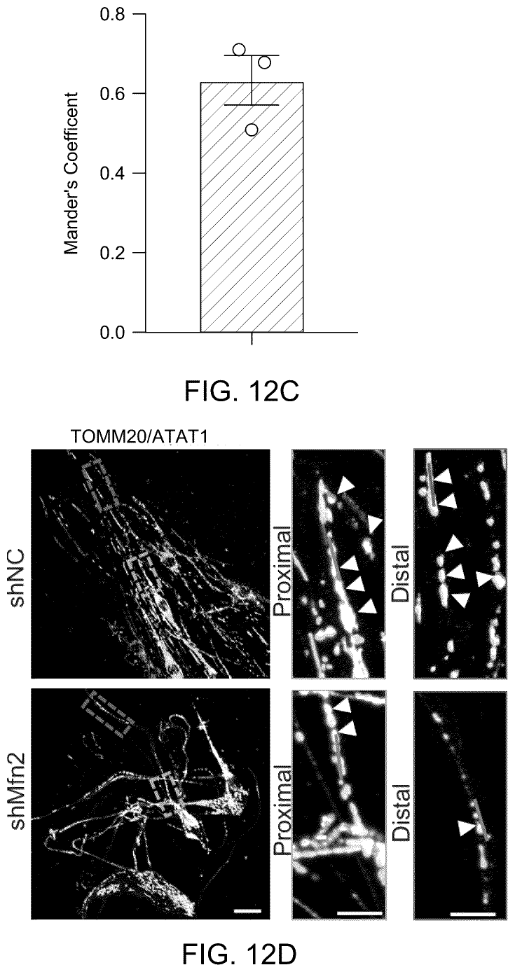

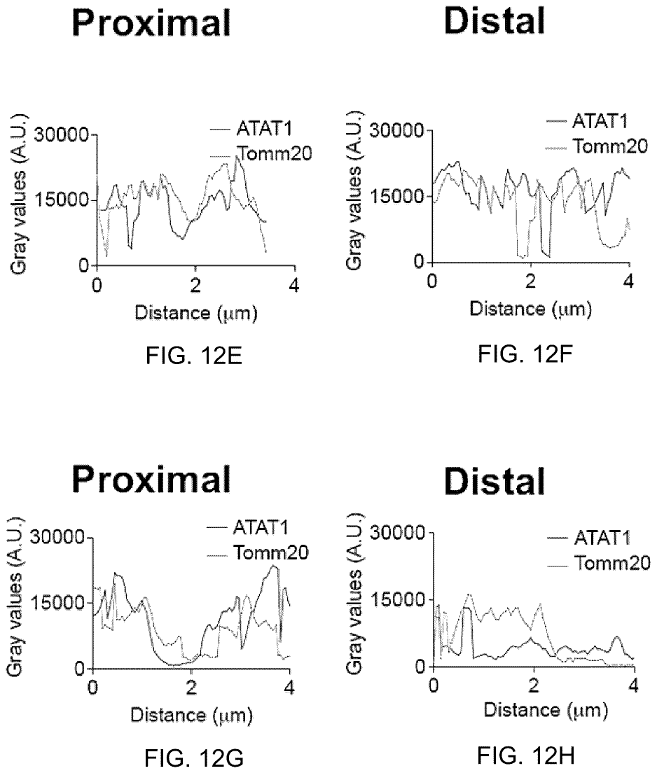

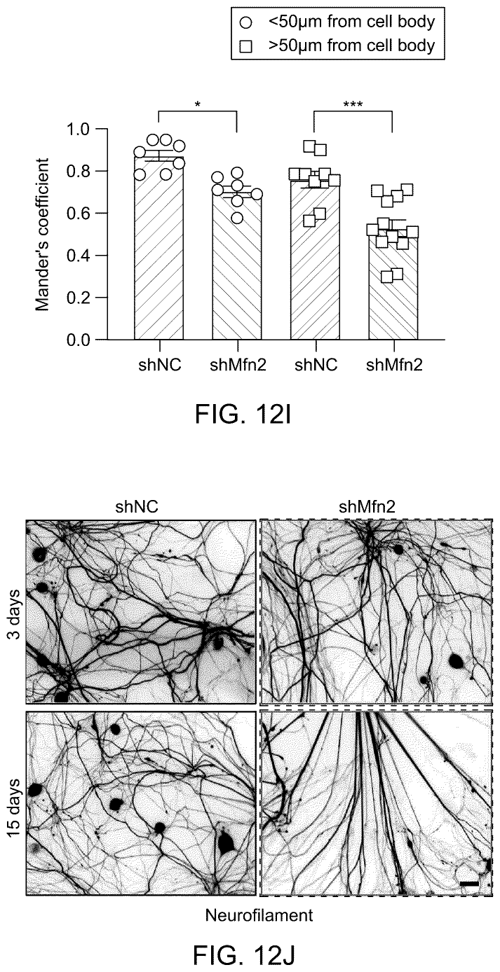

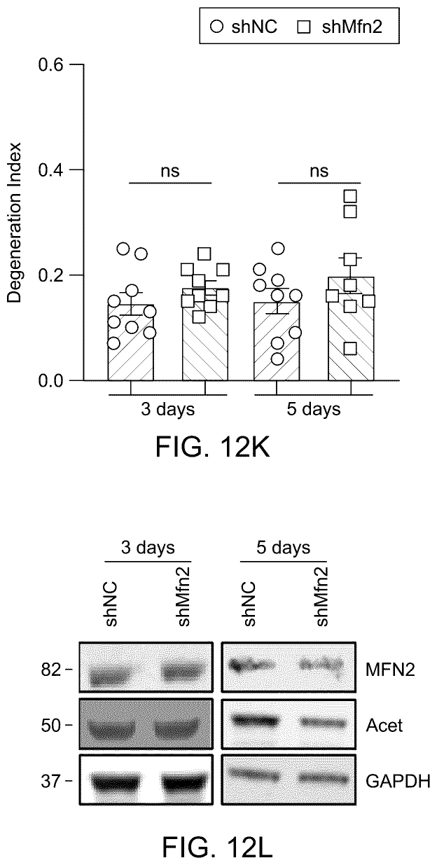

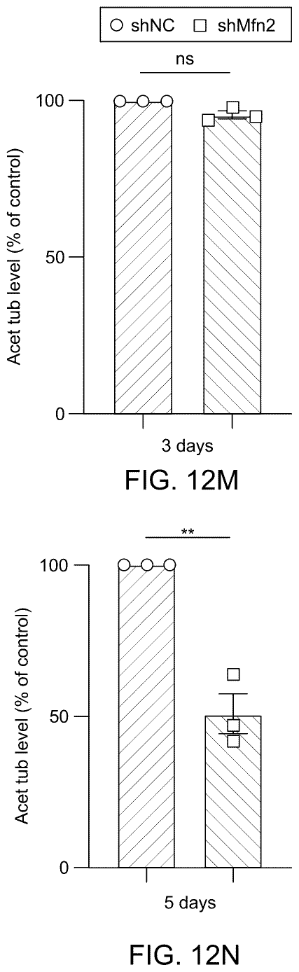

Antibodies

| | | |
|---|---|---|
| Mouse monoclonal mitofusin-2 (6A8) | Abcam | Cat# ab56889; |
| | | RRID:AB_2142629 |
| Rabbit monoclonal mitofusin-2 | Abcam | Cat# AB_124773; |
| | | RRID:AB_10999860 |
| Mouse monoclonal mitofusin-2 | Proteintech | Cat# 67487-1-Ig; |
| | | RRID:AB_2882713 |
| Rabbit polyclonal IQGAP1 | Novus Biologicals | Cat# NBP1-06529; |
| | | RRID:AB_1582398) |
| Rabbit polyclonal HDAC6 | Novus Biologicals | Cat# NBP1-78981; |
| | | RRID:AB_11037211 |
| Rabbit polyclonal ATAT1 | Bioss | Cat#Bs-9535R |
| Rabbit polyclonal ATAT1 (C6orf134) (For | Proteintech | Cat# 28828-1-AP; |
| FIGS. 7 and 12) | | RRID:AB_2881219 |
| Rabbit monoclonal TOMM20 | Abcam | Cat# AB_186735; |
| | | RRID:AB_2889972 |
| Mouse monoclonal TOMM20 | Millipore-Sigma | Cat#KB061-4F3 |
| Rabbit polyclonal detyrosinated tubulin | Abcam | Cat#ab48389; |
| | | RRID:AB_869990 |
| Rat tyrosinated tubulin, clone YL1/2 | Millipore | Cat# MAB1864; |
| | | RRID:AB_2210391 |
| Mouse monoclonal acetylated tubulin | Sigma Aldrich | Cat# T6793; |
| | | RRID:AB_477585 |
| Mouse monoclonal DM1A | Sigma Aldrich | Cat# T6199; |
| | | RRID:AB_477583 |
| Human anti-tubulin GTP (MB11) | Adipogen | Cat# AG-27B-0009; |
| | | RRID:AB_2490499 |
| Rabbit polyclonal TTL | Proteintech | Cat# 13618-1-AP; |
| | | RRID:AB_2256858 |
| Mouse monoclonal PDI | Santacruz | Cat# sc-74551; |
| | | RRID:AB_2156462 |
| Mouse monoclonal anti-c-Myc 9E10 HRP | Santacruz | Cat#SC-40 HRP |
| Mouse monoclonal anti-c-Myc 9E10 | Santacruz | Cat# sc-40 AC; |
| Agarose | | RRID:AB_2857941 |
| Mouse monoclonal anti-Flag M2 HRP | Sigma Aldrich | Cat# A8592; |
| | | RRID:AB_439702 |
| Mouse monoclonal anti-Flag M2 Agarose | Sigma Aldrich | Cat# A2220, |
| | | RRID:AB_10063035 |
| Chicken polyclonal neurofilament | Aves labs | Cat# AB_2313553 NFL; |
| | | RRID:AB_2313553 |
| Mouse monoclonal Miro-1(CL1083) | Abcam | Cat#Ab188029 |
| Rabbit polyclonal acetyl-Miro1 (Lys105) | Millipore | Cat#ABS2247 |
| Rabbit polyclonal anti-Miro2 | Proteintech | Cat#11235-1-AP |
| Rabbit polyclonal anti-Kif5c | Proteintech | Cat# 25897-1-AP; |
| | | RRID:AB_2880288 |
| Mouse monoclonal Cox4 | ThermoFisher | Cat# MA5-15078; |
| | | RRID:AB_10987478 |
| Mouse monoclonal GAPDH | Abcam | Cat# ab8245; |
| | | RRID:AB_2107448 |
| Rabbit polyclonal GAPDH | ThermoFisher | Cat# PA1-987; |
| | | RRID:AB_2107311 |
| Goat anti-mouse IgG (H + L) highly cross- | ThermoFisher | Cat#A11029 |
| adsorbed secondary antibody, Alexa Flour | | |
| 488-conjugated | | |
| Goat anti-rabbit IgG (H + L) highly cross- | ThermoFisher | Cat# A-11034; |
| adsorbed secondary antibody, Alexa Flour | | RRID:AB_2576217 |
| 488-conjugated | | |
| Goat anti-mouse IgG (H + L) highly cross- | ThermoFisher | Cat# A-11030; |
| adsorbed secondary antibody, Alexa Flour | | RRID:AB_2737024 |
| 546-conjugated | | |
| Goat anti-rabbit IgG (H + L) highly cross- | ThermoFisher | Cat# A-11035; |
| adsorbed secondary antibody, Alexa Flour | | RRID:AB_2534093 |
| 546-conjugated | | |
| Goat anti-human IgG (H + L) highly cross- | ThermoFisher | Cat# A-21090; |
| adsorbed secondary antibody, Alexa Flour | | RRID:AB_2535746 |
| 568-conjugated | | |
| IRDye_680RD Goat anti-mouse IgG | LI-COR | Cat# 926-68070; |
| secondary antibody | | RRID:AB_10956588 |
| IRDye_800CW Goat anti-rabbit IgG | LI-COR | Cat# 926-32211; |
| secondary antibody | | RRID:AB_621843 |
| IRDye_680RD Goat anti-rabbit IgG | LI-COR | Cat# 926-68071; |
| secondary antibody | | RRID:AB_10956166 |
| IRDye_800CW Goat anti-rabbit IgG | LI-COR | Cat# 926-32211; |
| secondary antibody | | RRID:AB_621843 |

Bacterial strains

| | | |
|---|---|---|
| DH5 alpha | New England | Cat#C2987I |
| | Biolabs | |
| XL1-Blue | Agilent | Cat#200229 |

Chemicals, peptides, and recombinant proteins

| | | |
|---|---|---|
| Trichostatin A (TSA) | Tocris | Cat#1406 |
| Tubacin | Millipore-Sigma | Cat#SML0065 |
| Mitotracker Red CMXROS | Thermofisher | Cat#M7512 |
| DMEM | Gibco | Cat#11995-065 |
| Neurobasal | Thermofisher | Cat#110349 |
| Fetal bovine serum | HyClone | Cat#SH30071.03 |
| Bovine calf serum | Thermofisher | Cat#26170043 |
| B-27 supplement (50x) | Thermofisher | Cat#7504044 |
| Penicillin-streptomycin | Thermofisher | Cat#15140163 |
| 10x HBSS | Thermofisher | Cat#14065056 |
| Cytosine b-D-arabinofuranoside | Millipore-Sigma | Cat#C6645 |
| hydrochloride (AraC) | | |
| Collagenase | Millipore-Sigma | Cat#C0130 |
| Trypsin 0.05% EDTA | Thermofisher | Cat#25300054 |
| GlutaMAX Supplement | Thermofisher | Cat#35050061 |
| Poly-D-Lysine | Sigma Aldrich | Cat#P1149 |
| Laminin | Sigma Aldrich | Cat#11243217001 |
| Laemlli SDS sample buffer, reducing | Thermofisher | Cat#J60015-AD |
| NuPAGE MOPS SDS Running buffer | Thermofisher | Cat#NP0001 |
| Fluoromount-G | Southern Biotech | Cat#0100-01 |
| 32% PFA | EMS | Cat#15714-S |
| DreamFect_Gold transfection reagent | OZ Bioscience | Cat#DG80500 |
| c-Myc Peptide | Sigma Aldrich | Cat#M2435 |
| Flag peptide | Sigma Aldrich | Cat#F3290 |

Commercial assays

| | | |
|---|---|---|
| DuoLink PLA In Situ Red starter | Sigma-Aldrich | DUO92101 |
| mouse/rabbit kit | | |

Experimental models: Organisms/strains

| | | |
|---|---|---|
| UAS-MARF RNAi | Bloomington Stock | BL 31157 |
| | Center | |
| UAS-MARF miRNA CDS | Bloomington Stock | BL 67158 |
| | Center | |
| Mouse: C57BL/6J | Charles River | RRID:IMSR CRL:027 |
| | Laboratories | |

Recombinant DNA

| | | |
|---|---|---|
| pLKO.1 shMFN2 | Sigma Aldrich | TRCN0000080608 |
| pLKO.1 shTTL | Sigma Aldrich | TRCN0000191515 |
| MFN2 7X Myc in pCLBW | Detmer and Chan | NA |
| MFN2 R94W 7X Myc in pCLBW | Detmer and Chan | NA |
| MFN2 T105M 7X Myc in pCLBW | Detmer and Chan | NA |
| Flag-ATAT1 WT | Laurent Ngyuen | NA |
| Flag-ATAT1 (1-242) | Laurent Ngyuen | NA |
| Flag-ATAT1 (1-286) | Laurent Ngyuen | NA |
| Zsgreen-MFN1 | Estella Area Gomez | NA |
| Mito-dendra2 | Addgene | 55796 |
| pLV OMP25 (rtOMP25; NM 022599.2; | VectorBuilder | VB220328-1304ynx |
| Promoter: SYN1; | See Tables 6 and 7 | |
| Linker: IRES; Fluorescent tag: EmGFP) | | |
| pLV hATAT1-OMP25 [hATAT1 | VectorBuilder | VB220329-1381pbx |
| (isoform#2, NM_024904.5) | See Tables 6 and 7 | |
| rtOMP25 C-terminal; Promoter: SYN1; | | |
| Linker: IRES; Fluorescent tag: EmGFP] | | |

Software

| | | |
|---|---|---|
| ImageJ (Fiji) | NIH | RRID:SCR_002285 |
| GraphPad Prism | GraphPad | RRID:SCR_002798 |
| LI-COR Image Studio Software | LI-COR | RRID:SCR_015795 |
| Zeiss ZEN | Zeiss | RRID:SCR_013672 |
| Andor iQ3 | Oxford Instruments | RRID:SCR_014461 |

Other

| | | |
|---|---|---|
| 18 mm No. 1 circle coverglass | Carolina | 633033 |
| 35 mm MatTek dishes | MatTek | P35G-1.5-14-C |
| NuPAGE Gel | ThermoFisher | NP0316 |
| Nitrocellulose membrane | Fisher | 10600011 |

### 6.1.1 Mice for Primary Cell Cultures

All protocols and procedures for mice were approved by the Committee on the Ethics of Animal Experiments of Columbia University and according to Guide for the Care and Use of Laboratory Animals of the National Institutes of Health. All experiments with mice to generate adult DRG cultures were sex balanced and no influence of gender was observed.

6.1.2. Drosophila melanogaster

All animals were randomly selected regardless of sex.

### 6.1.3. Cell Lines

WT, MFN2 KO, MFN1 KO (kind gifts of Dr. Area-Gomez) and Iqgap1 KO mouse embryonic fibroblast cells were grown in DMEM supplemented with 10% fetal bovine serum. Cells were grown to 80% confluency on acid treated glass coverslips prior to experiment. Cells were transfected using DreamFect™ Gold transfection reagent according to manufacturer's protocol. 1-2 ug of DNA were diluted in low serum DMEM medium and 2-4 μl of DreamFect Gold transfection reagent. Solution was incubated for 20 min at R.T. and added in cells and incubated overnight at 37° C. All cell lines have been authenticated through western blot analysis using specific antibodies and all the experiments were conducted using mycoplasma-free cells, which was monitored using PCR methods.

### 6.1.4. Confocal Microscopy and Analysis

Cells were fixed in 4% PFA for 15 min and permeabilized with 0.1% Triton X-100 for 5 min at R.T. For the MT cytoskeleton, cells were fixed in ice cold MetOH for 10′ prior to rehydration in PBS buffer. Cells were then washed in PBS, blocked in 2% FBS and 2% BSA in PBS for 1 h, stained with primary antibodies overnight at 4° C. followed by secondary antibodies for 1 h. Mounted samples were observed using a Zeiss LSM 800 confocal microscope using a 63× objective (Plan-Apochromat, NA 1.4). Z stack (0.5 um) images were acquired. To calculate the Mitotic Index as percentage, the number of cells in mitosis were divided by the total number of cells multiplied by 100. To calculate ratio of acetylated/tyrosinated tubulin intensity, each file was converted to 8-bit greyscale and maximum projection images were generated by using ImageJ. ROIs were selected in both channels to calculate mean fluorescence and integrated density prior to calculating corrected total fluorescence intensity (CTCF=Integrated Density−(Area of selected cell*Mean fluorescence of background readings). For measuring MT associated MB11 intensity, an ROI was selected along individual tyrosinated MTs using ImageJ and GTP tubulin (hMB11) pixel intensity was calculated along the tyrosinated tubulin signal and corrected by subtracting out background signal fluorescence intensity.

### 6.1.5. Airyscan Confocal Microscopy and Analysis

For immunofluorescence staining of the MT cytoskeleton, cells were fixed in ice cold MetOH for 10′ prior to rehydration in PBS buffer. For all other staining, cells were fixed in 4% PFA for 15 min and permeabilized with 0.1% Triton X-100 for 5 min at R.T. Cells were then washed in PBS, blocked in 2% FBS and 2% BSA in PBS for 1 h, stained with primary antibodies overnight at 4° C. followed by secondary antibodies for 1 h. Mounted samples were observed using a Zeiss LSM 800 confocal microscope equipped with Airyscan module, using a 63× objective (Plan-Apochromat, NA 1.4). Z stack (0.2 um) images were acquired and processed using Zen Blue 2.1 software prior to analysis by ImageJ software. For 3D reconstruction, mitochondrial networks in individual cells were quantified by analyzing the network volume using Imaris software (Bitplane, Concord, MA). For line scan analysis, a line was drawn, and intensity was measured represented as gray values. The amount of fluorescence of the colocalizing pixels in each channel (Mander's coefficient) was calculated using ImageJ. The file was opened, and channels were split. Each select file was changed to 8-bit greyscale and max projection images was generated. Colocalization was assessed by Plugin co-localization finder. To calculate number of ATAT1 foci, each image was converted into an 8-bit greyscale image and ATAT1 signal thresholded until the foci appeared isolated from the background in a binary image. Both channels were then merged into one and the number of foci localizing in the merged stack quantified. To calculate ATAT1 localization with MFN2 on mitochondria, numbers of ATAT1 and MFN2 foci were quantified on each channel individually. Both channels were then merged into one and number of foci in the merged stack analyzed and quantified on mitochondria.

### 6.1.6. Western Blot Analysis

Cells were lysed in Laemmli sample buffer and boiled at 96° C. for 5 min. Cell lysates were sonicated with a probe sonicator to sheer cellular debris and genomic DNA. Proteins were separated by 10% Bis-Tris gel (Invitrogen) and transferred onto nitrocellulose membrane. After blocking in 5% milk/TBS or BSA/TBS, membranes were incubated with primary antibodies at 4° C. overnight prior to 1 h incubation with secondary antibodies. Image acquisition was performed with an Odyssey imaging system (LI-COR Biosciences, NE) and analyzed with Odyssey software.

### 6.1.7. Proximity Ligation Assay (PLA)

PLA assays were carried out using a Duolink® in situ red starter kit mouse/rabbit kit (Sigma-Aldrich) according to the manufacturer's protocol. The primary antibodies used were mouse anti-MFN2 (1:100 dilution), anti-ATAT1 (1:50 dilution), anti-Miro1 (1:50 dilution). Z stack images were acquired on a Zeiss LSM 800 confocal microscope using a 63× objective (Plan-Apochromat, NA 1.4) and puncta were analyzed using ImageJ/FIJI. Each image was processed and the channel containing the PLA signal was thresholded until the PLA puncta were reliably isolated from the background forming a binary image. Overlapping PLA puncta were segmented using a watershed function. Size thresholding was adjusted, and the number of PLA puncta were then counted in each cell.

### 6.1.8. Analysis of Mitochondrial Morphology, Motility, and Distribution

Mitochondria were labeled using MitoTracker™ Red CMROX according to manufacturer protocol (ThermoFisher Scientific) and detected by epifluorescence microscope equipped with 60× objective lens (Olympus IX81) and a monochrome CCD camera (Sensicam QE, Cooke Corporation). Aspect ratio (length/width) were measured in unedited image files by using Image J/FIJI. Brightness and contrast of the images were adjusted to optimize visualization of the mitochondrial segments and to reduce background noise. After selecting an individual cell, the Analyze Particles function (pixel size 10-infinity; circularity 0.00-1.00) was used to count the number of noncontiguous, discrete particles, and describe their shape. For mitochondrial distribution and displacement velocity mitochondria were live imaged for 3 min at 2 sec/frame at 37° C. A customized Mitoplot software was used for analyzing mitochondrial distribution by defining the center of cell and drawing a line towards periphery. Manual tracking plug-in in ImageJ/FIJI was used to analyze mitochondrial displacement velocity by selecting individual mitochondria.

### 6.1.9. Microtubule Dynamics

Fibroblasts were transfected with pMSCV-puro-tagGFP-C4 α-tubulin plasmid to generate a green fluorescent protein (GFP)-tubulin stably expressing cell line. Live imaging of MT dynamics in transfected cells was performed at 37° C. and 5% CO₂ for 5 min (5 s/f) with a 100×

PlanApo objective (numerical aperture 1.45) and an iXon X3 CCD camera (Andor, Belfast, United Kingdom) on a Nikon Eclipse Ti microscope controlled by Nikon's NIS-Elements software (Nikon, Tokyo, Japan). Movies were analyzed by ImageJ using a manual tracking plug-in. Dynamicity is calculated by dividing the sum of growth and shrinkage distances by MT lifetime.

### 6.1.10. Mitodendra

Dendra2 photoconversion and imaging utilized the protocol from Evrogen. Images were acquired with an Olympus spinning disk microscope EC-Plan-Neofluar 40X/1.3 oil. Z-stack acquisitions over-sampled each optical slice twice, and the Zen 2009 image analysis software was used for maximum z-projections. The 488 nm laser line and the 561 nm laser excited Dendra2 in the unconverted state and photo-converted state, respectively. To photo-switch Dendra2, a region was illuminated with the 405 nm line (4% laser power) for 90 bleaching iterations. The corrected total fluorescence intensity of both the channels was calculated using ImageJ and colocalization percentage was calculated.

### 6.1.11. Lentivirus Production

Production of lentiviral particles was conducted using the second-generation packaging system (see Pero et al. (2021) Proc Natl Acad Sci USA 118. ARTN e2012685118; and Qu et al. (2019) Curr. Biol. 29, 4231-4240. In brief, HEK293T cells were co-transfected with lentiviral plasmid shRNA and the packaging vectors pLP1, pLP2, and pLP-VSV-G (ThermoFisher) using the Ca2+ phosphate transfection method. At 24, 36, and 48 h after transfection, the virus-containing supernatant was collected, and the lentiviral particles concentrated (800-fold) by ultracentrifugation (100,000×g at 4° C. for 2 h) prior to aliquoting and storage at −80° C.

### 6.1.12. Retrovirus Production

MFN2 Myc, MFN2 R94W Myc and MFN2 T105M Myc cDNAs were cloned into the retroviral construct pCLBW and the retroviral particles generated as detailed (see Detmer and Chan, (2007) J. Cell Biol. 176, 405-414 and Chen et al. (2003) J. Cell Biol. 160, 189-200). Briefly, retroviral expression vectors were co-transfected with the ecotropic retroviral packaging vector pCLEco into 293T cells. 48 h after transfection retroviral particles were harvested by pelleting cells and debris at 900×g for 5 min. Supernatants were passed through 0.45 mm syringe filters, aliquoted and immediately frozen on dry ice prior to storing at −80° C.

### 6.1.13. MT Stability

WT and MFN2 KO MEFs were incubated at 8° C. for 30 min to induce mild MT depolymerization. At the end of the incubation time, cells were gently washed with PEM 13 buffer (85 mM Pipes, pH 6.94, 10 mM EGTA, and 1 mM MgCl2) twice before extraction with PEM buffer, supplemented with 0.05% Triton X-100. After 1 min extraction at 8° C., a matching volume of fixative buffer (ice cold MetOH) was added dropwise to the coverslips, and cells were incubated for another 5 min at −20° C. Cells were finally washed with 1×PBS and processed for immunofluorescence labeling. Mounted samples were observed using a Zeiss LSM 800 confocal microscope using a 63× objective (Plan-Apochromat, NA 1.4). Z stack (0.5 um) images were acquired and processed using Zen Blue 2.1 software. All images were analyzed using ImageJ software as described (see Khawaja et al., (1988) J. Cell Biol. 106, 141-149). Each select file was changed to 8-bit greyscale and max projection images were generated. Using Image J area was selected in to calculate corrected total fluorescence intensity for DM1A tubulin after extraction and normalized to total DM1A tubulin intensity.

### 6.1.14. Analysis of Phospholipid Synthesis in Cultured Cells

Both mitochondria and ER play key roles in the synthesis of phosphatidylserine (PtdSer), phosphatidylethanolamine (PtdEtn), and phosphatidylcholine (PtdCho). PtdSer is synthesized in the MAM; it then translocates to mitochondria, where it is converted to PtdEtn; PtdEtn then translocates back to the MAM, to generate PtdCho. To test the effect of MFN2 on phospholipid synthesis mediated by MAM, WT and MFN2 KO MEF cells were incubated for 2 h with serum-free medium to ensure removal of exogenous lipids. The medium was then replaced with MEM containing 2.5 mCi/ml of 3H-serine for 2, 4 and 6 h. The cells were washed and collected in DPBS, pelleted at 2500×g for 5 min at 4° C., and resuspended in 0.5 ml water, removing a small aliquot for protein quantification. Lipid extraction was done by the Bligh and Dyer method. Briefly, three volumes of chloroform/methanol 2:1 were added to the samples and vortexed. After centrifugation at 8000 g for 5 min, the organic phase was washed twice with two volumes of methanol/water 1:1, and the organic phase was blown to dryness under nitrogen. Dried lipids were resuspended in 60 ml of chloroform/methanol 2:1 (v/v) and applied to a TLC plate. Phospholipids were separated using two solvents, composed of petroleum ether/diethyl ether/acetic acid 84:15:1 (v/v/v) and chloroform/methanol/acetic acid/water 60:50:1:4 (v/v/v/v). Development was performed by exposure of the plate to iodine vapor. The spots corresponding to the relevant phospholipids (identified using co-migrating standards) were scraped and counted in a scintillation counter (Packard Tri-Carb 2900TR). Both mitochondria and ER play key roles in the synthesis of phosphatidylserine (PtdSer), phosphatidylethanolamine (PtdEtn), and phosphatidylcholine (PtdCho). PtdSer is synthesized in the MAM; it then translocates to mitochondria, where it is converted to PtdEtn; PtdEtn then translocates back to the MAM, to generate PtdCho. Therefore, to directly test the effect of MFN2 mutations on phospholipid synthesis mediated by MAM, control and MFN2 KO fibroblasts were incubated in medium containing 3H-serine and measured the incorporation of the label into newly synthesized 3H-PtdSer and 3H-PtdEtn after 2 and 4 h.

### 6.1.15. Lipidomics

All samples were collected and treated following recently accepted guidelines for the analysis of human blood plasma and/or serum. Lipids were extracted from equal amounts of material (0.2 ml/sample) by a chloroform-methanol extraction method. Three comprehensive panels, scanning for either positive lipids, negative lipids, or neutral lipids (under positive mode), were analyzed. Equal amounts of internal standards with known concentrations were spiked into each extract. Each standard was later used to calculate the concentrations of corresponding lipid

classes by first calculating ratio between measured intensities of a lipid species and that of corresponding internal standard multiplied by the known concentration of the internal standard. Samples were analyzed using a 6490 Triple Quadrupole LC/MS system (Agilent Technologies, Santa Clara, CA). Cholesterol and cholesterol esters were separated with normal-phase HPLC using an Agilent Zorbax Rx-Sil column (inner diameter 2.1 Å˜100 mm) under the following conditions: mobile phase A (chloroform:methanol:1M ammonium hydroxide, 89.9:10:0.1, v/v/v)
and mobile phase B (chloroform:methanol:water:ammonium hydroxide, 55:39.9:5:0.1, v/v/v/v); 95% A for 2 min, linear gradient to 30% A over 18 min and held for 3 min, and linear gradient to 95% A over 2 min and held for 6 min.

### 6.1.16. Cellular Fractionation

WT and MFN2 KO fibroblasts were cultured on 15 cm petri dishes. Buffer A (10 mM HEPES, 1.5 mM MgCl2, 10 mM KCl, 0.5 mM DTT, 0.05% NP40, pH 7.9) was prepared freshly and protease and phosphatase inhibitors were added. Cells were scraped thoroughly using 1 ml of buffer A and left on ice for 10 min. Samples were centrifuged at 3000 rpm on a table top centrifuge for 10 min at 4° C. and supernatants were stored on ice. Pellets were resuspended in buffer B (5 mM HEPES, 1.5 mM MgCl2, 0.2 mM EDTA, 0.5 mM DTT, 26% Glycerol (v/v), pH 7.9) and 1 μl of 4.6M NaCl was added to a final concentration of 4.6 mM. Cells were homogenized with 20 full strokes of Dounce homogenizer on ice and left on ice for 30 min. Samples were centrifuged at 3000 rpm for 10 min at 4° C. Pellet contains the nuclear fraction and supernatant contains the cytosolic fraction. For the mitochondrial fraction assay, a mitochondrial isolation kit was used (Thermo Scientific, 89874). WT and MFN2 KO fibroblasts were cultured on 10 cm petri dishes. Cells were pelleted by centrifuging the cell suspension. Protease inhibitors were added to reagent A and C immediately before use. 800 ml of mitochondrial isolation reagent A was added, cells vortexed at medium speed for 5 s and incubated on ice for exactly 2 min. 10 ml of mitochondrial isolation reagent B was added, before vortexing at maximum speed for 5 s and incubating on ice for 5 min with vortexing at every min. 800 ml of Mitochondrial reagent C was added and tubes were inverted several times to mix. Tubes were centrifuged at 700×g for 10 min at 4° C. and supernatants transferred and centrifuged at 12,000×g for 15 min at 4° C. and 3,000×g for 15 min. Pellets contain the mitochondria fraction. All the samples were processed for western blot analysis.

### 6.1.17. Isolation of Adult DRG Neurons

Dorsal root ganglions (DRG) were dissected from 8 to 10-wk-old C57BL/6J mice in cold Hank's balanced salt solution (HBSS) (Life Technologies) or Dulbecco's Modified Eagle's medium (Life Technologies) and dissociated in 1 mg/mL collagenase A for 1 h at 37° C., followed by 0.05% trypsin (Life Technologies) digestion for 3 to 5 min at 37° C. and washed with Neurobasal medium (Invitrogen) supplemented with 2% B-27 (Invitrogen), 0.5 mM glutamine (Invitrogen), fetal bovine serum (FBS), and 100 U/mL penicillin-streptomycin. DRG neurons were then triturated by repeated gentle pipetting until no clump was visible, and neuronal bodies were resuspended in Neurobasal medium with FBS prior to plating onto 12 well plates (over 18 mm coverslips) that had been coated overnight with 100 mg/mL poly-D-lysine at 37° C. and for 1 h at 37° C. with 10 mg/mL laminin (Life Technologies). After 30 min, Neurobasal medium, without FBS, was added to the plate. At 4DIV, at least 30% of media was changed and 10 mM AraC was added to media every 4 d. DRG neurons were treated with lentiviral particles at 7DIV. For overexpression of OMP25 and

ATAT1 OMP25 lentiviral particles DRG neurons were infected at 7DIV for 5 days. Myc MFN2 R94W and Myc MFN2 T105M retroviruses were infected at 7 DIV for 5 days in DRG neurons.

### 6.1.18. Degeneration Index in DRG Neurons

Images of random fields of dissociated adult DRG neurons fixed with 4% PFA and immunostained with mouse anti-neurofilament antibody were acquired using a 203 objective lens (Olympus IX81) coupled to a monochrome CCD camera (Sensicam QE; Cooke Corporation). To quantify axonal degeneration, the areas occupied by the axons (total axonal area) and degenerating axons (fragmented axonal area) were measured in the same field from images of DRG neurons. Images were automatically thresholded (global threshold) using a default auto threshold method, binarized, and the fragmented axonal area measured by using the particle analyzer module of ImageJ (size of small fragments=20 to 10,000 pixels). Degeneration index was calculated as the ratio between the fragmented axonal area and the total axonal area.

### 6.1.19. Immunolabeling of Drosophila Larvae

Immunolabeling of Drosophila larvae was described in performed as in Shin et al. (Proc. Natl. Acad. Sci. USA, (2021) 118, e2006050118). Briefly, late third instar larvae were dissected in 1 3 PBS, fixed in 4% paraformaldehyde (PFA, Electron Microscopy Sciences) in 1×PBS for 15 min, washed three times in 1×PBS+0.3% Triton X-100 (PBS-TX), and blocked for 1 h at R.T. or overnight at 4° C. in 5% normal donkey serum (NDS) in PBS-TX (Jackson ImmunoResearch). Primary antibodies were chicken anti-GFP (1:1000; Abcam) and acetylated α-tubulin (1:400; Sigma Aldrich) diluted in 5% NDS in PBS-TX. The tissue was incubated overnight in primary antibodies at 4° C. and then washed in PBS-TX for 3×15 min at R.T. Species-specific, fluorophore-conjugated secondary antibodies (Jackson ImmunoResearch) were used at 1:1000 in 5% NDS in PBS-TX and incubated overnight 4° C. Tissue was washed in PBS-TX for 3×15 min. Immunolabeled tissue was mounted on poly-L-lysine coated coverslips, dehydrated 5 min each in an ascending ethanol series (30, 50, 70, 95, 2 3 100%), cleared in xylenes (2×10 min), and mounted in DPX (Fluka).

### 6.1.20. Imaging and Quantification of Drosophila Sensory Neurons

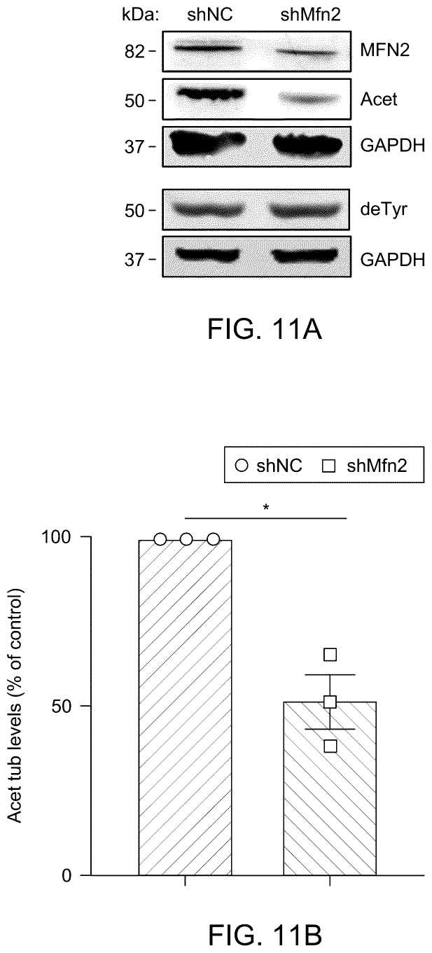

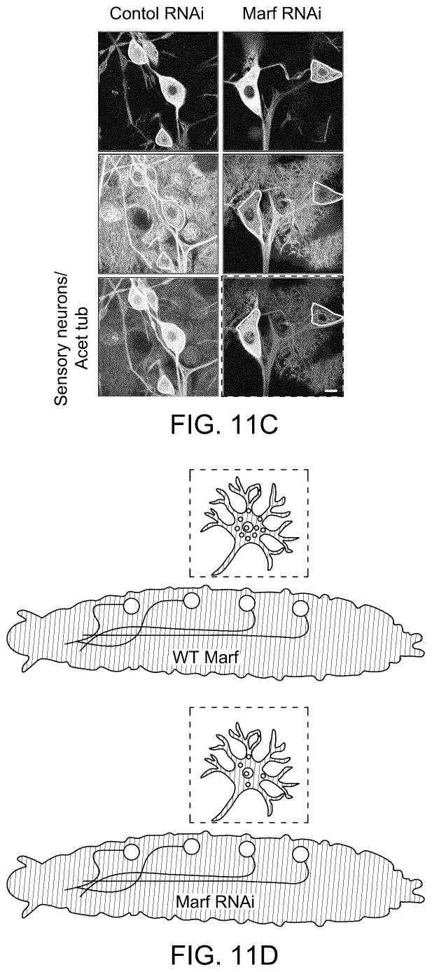

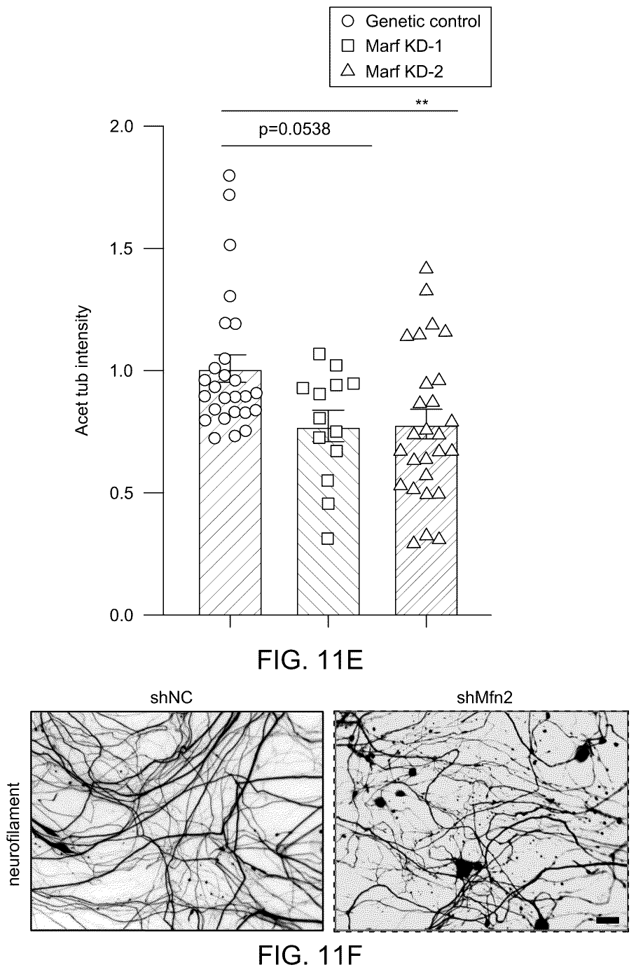

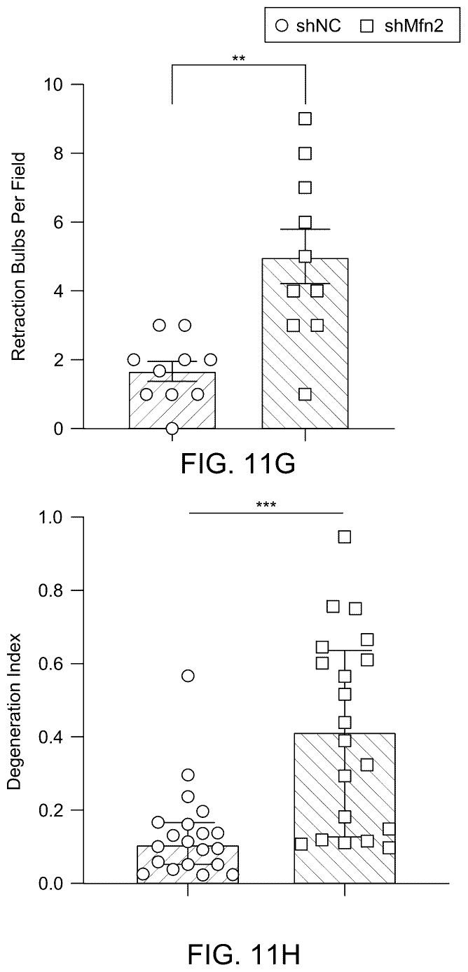

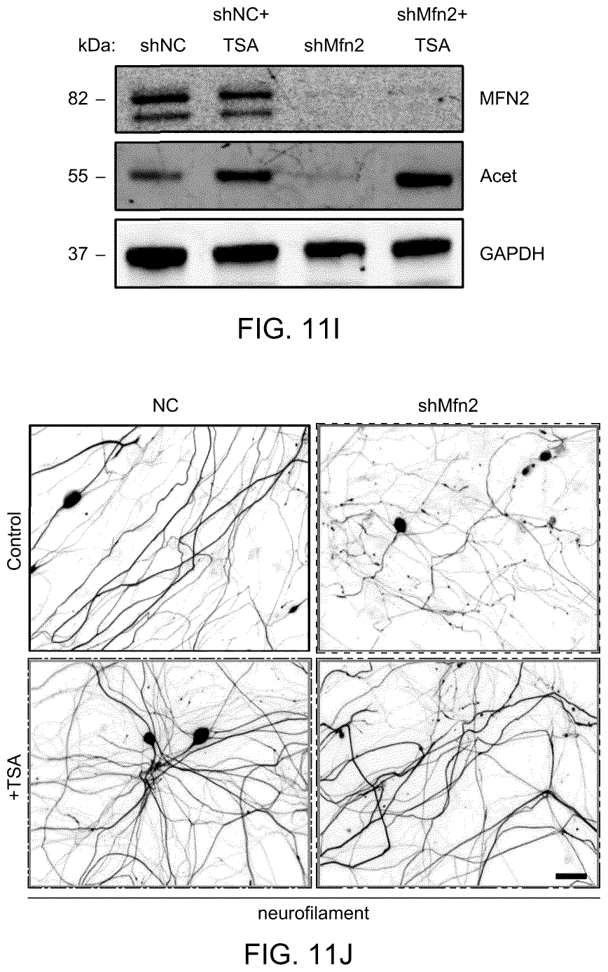

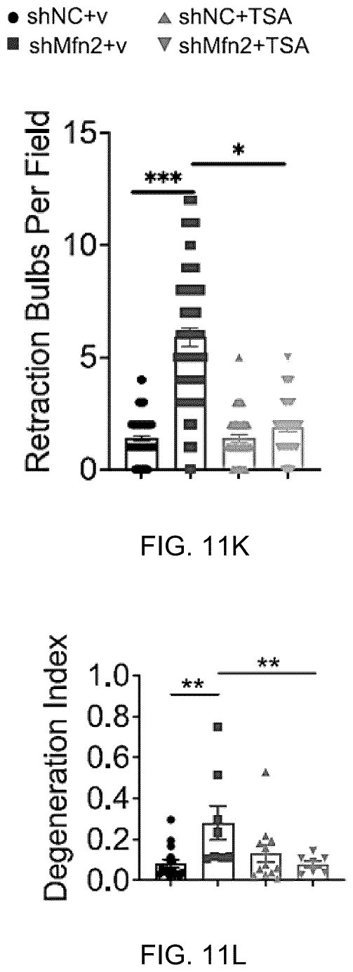

Images of somatosensory neurons from Drosophila larvae were acquired using a Yokogawa CSU-W1 SoRa mounted on a Zeiss Axio Observer using a 60×1.46 NA Alpha Plan-Apochromat oil objective and a 4× magnification changer. Acquisitions included the cell body, axon, and dendrites of somatosensory neurons. Subsequent image analysis was performed using Fiji. Using the md neurons (109(80)2-Gal4, UASCD8-GFP) as reference, sub stacks covering the z-depth of each cell body were cropped and blinded for subsequent analysis. Additionally, 2-3 areas (300×300 px) devoid of neurons in the same image were selected to measure background levels of acetylated tubulin in each image and used to normalize the levels in the cell body. To measure acetylated tubulin levels in the cell bodies, cell bodies were selected using the polygon selection tool, and the area outside the cell body was cleared to avoid including acetylated tubulin staining surrounding the cell body in the subsequent quantification. Processed z-stacks of cell bodies were z-projected using average intensity. The mean gray value was measured and normalized against background levels of acetylated tubulin quantified in the same image. Raw images were used for quantification. Represented images shown in FIG. 11C were deconvolved using Microvolution (20 iterations).

### 6.1.21. Co-Immunoprecipitation Assay

HEK293T cells were cultured in Dulbecco's modified Eagle's medium plus 10% fetal bovine serum (FBS), penicillin-streptomycin (1%) and L-glutamine (1%). Transient transfections were performed using DreamFect_Gold transfection reagent (Oz Biosciences SAS, Marseille, FR) in accordance with the manufacturer's protocols. HEK293T cells were lysed in RIPA buffer (50 mM Tris-HCl at pH 7.6, 150 mM NaCl, 0.5% sodium deoxycholic, 5 mM EDTA, 0.1% SDS, 100 mM NaF, 2 mM NaPPi, 1% NP-40) supplemented with protease and phosphatase inhibitors, then centrifuged at 13,000 rpm for 30 min at 4° C. and the resulting supernatants were subjected to Bradford protein assay (Bio-Rad, Hercules, California, USA) for measuring total protein concentration. Immunoprecipitations were performed on 1 mg of whole cell extracts by using anti-c-Myc agarose conjugated or anti-Flag M2 agarose (1-2 mg) beads for 2 h at 4° C. with rotation. c-Myc peptide or Flag-peptide (0.1 mg/ml) was used as a control. The immunoprecipitated complexes were then washed 5× with RIPA buffer, resuspended in sample loading buffer, boiled for 5 min, resolved by SDS-PAGE, and then subjected to immunoblot analysis.

### 6.1.22. Quantification and Statistical Analysis

Data distributions that did not pass the normality tests are shown as medians plus interquartile range, and statistical significance was analyzed by nonparametric unpaired two-tailed Mann-Whitney U test or Kruskal-Wallis with Dunn's multiple comparisons test. Data distributions that passed the normality test are shown as mean±SEM, and statistical significance was analyzed by student's t test and two-way ANOVA with Dunnett's multiple comparison. GraphPad prism was used to plot graph. Description of n (samples) or N (population) values are indicated in each figure legend. All experiments were repeated at least three times unless stated otherwise in each figure legend.

### 6.2. Example 2. MFN2 is a Novel Regulator of Tubulin Acetylation

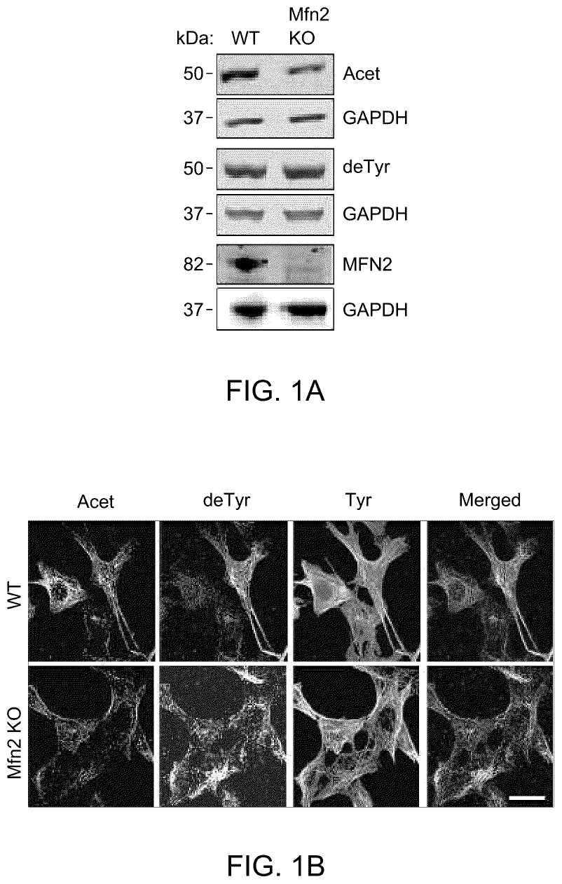

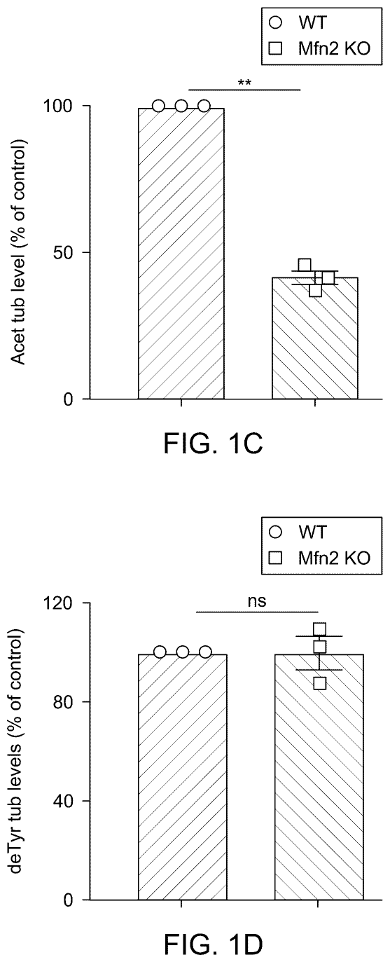

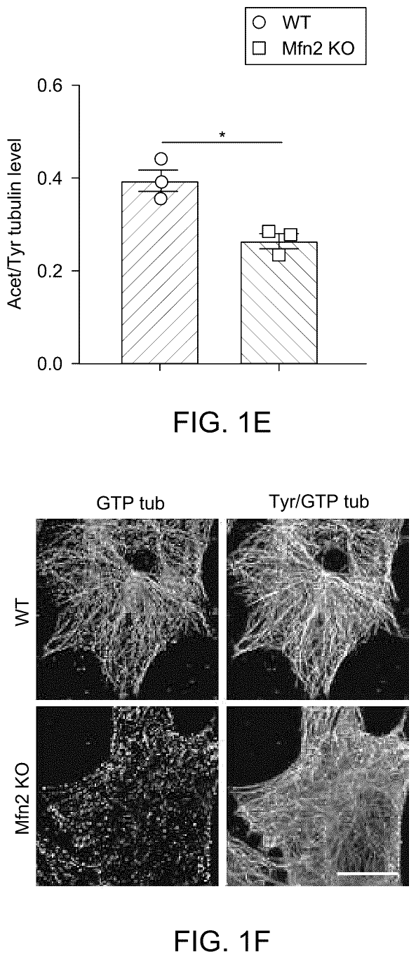

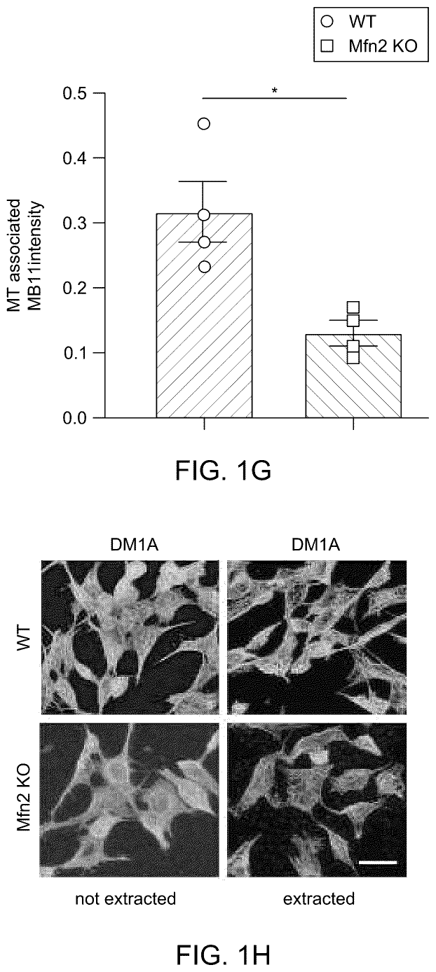

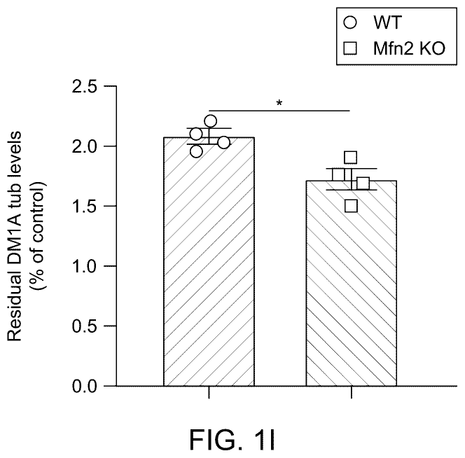

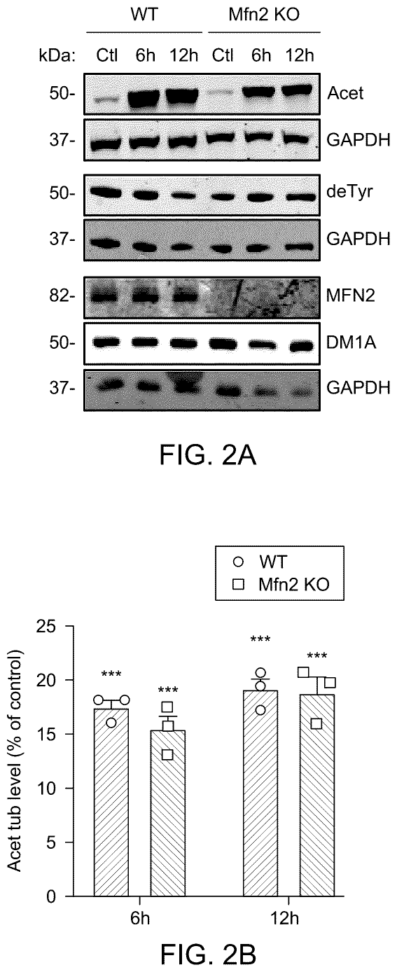

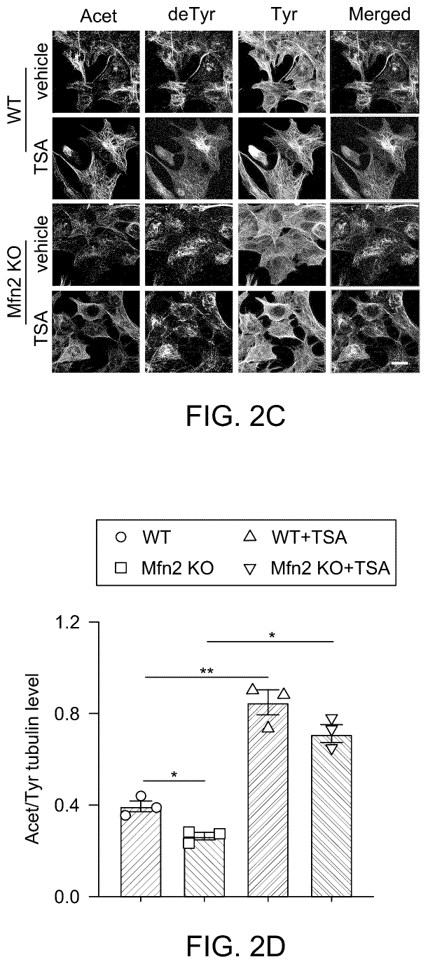

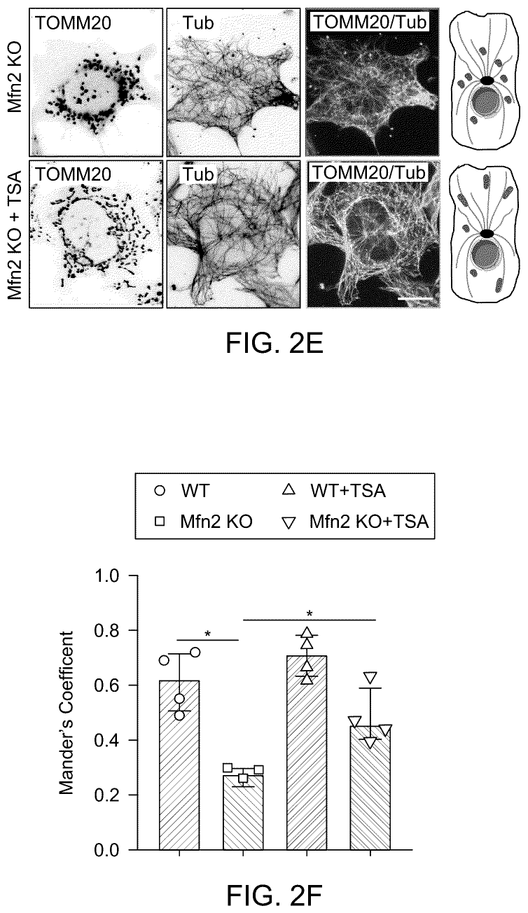

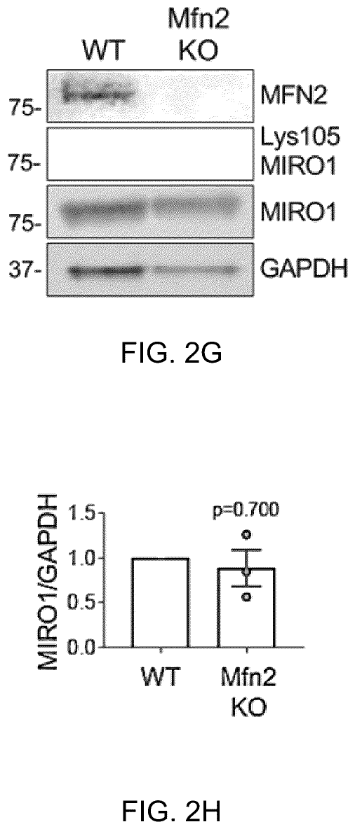

To test whether the machinery controlling mitochondria motility and/or hetero-homotypic mitochondrial contacts functionally interact with the α-tubulin acetylation cycle, levels of acetylated α-tubulin were measured in immortalized MFN2 KO mouse embryonic fibroblast (MEF) cells having defects in mitochondria dynamics and functional tethering with the ER (de Brito et al., Nature, 2008. 456, 605-10). Immunoblot and immunofluorescence analyses showed that while de-tyrosinated tubulin levels were unaffected, loss of MFN2 reduced acetylated tubulin by more than 50% compared to WT controls (FIGS. 1A-1E and FIGS. 2A-D). Loss of acetylated tubulin in these cells also correlated with a decrease in the abundance of GTP-tubulin islands, the putative entry sites for the tubulin acetyltransferase ATAT1 into the MT lumen and hotspots of MT self-repair by incorporation of GTP-bound tubulin subunits (FIGS. 1F and 1G).

MT plus end dynamics was measured by following the behavior of individual MTs in WT and MFN2 KO cells transfected with GFP-tubulin and found that lack of MFN2 expression almost doubled MT dynamicity, an effect due to an increase in MT growth and shrinkage rates (Table 2). The rise in MT dynamicity correlated with a significant loss of MT stability. To test this, the amount of residual MT polymer resisting depolymerization that was induced by mild detergent extraction prior to fixation and immunofluorescence staining was measured (FIGS. 1H and 1I). Importantly, both acetylated tubulin levels and MT dynamics were normalized in MFN2 KO cells by the HDAC class I/II inhibitor trichostatin A (TSA) (FIGS. 2A-2D and Table 3), suggesting that the increase in MT dynamicity resulted from loss of α-tubulin acetylation in cells deprived of MFN2.

TABLE 2
MFN2 regulates MT dynamics in MEFs.

| Parameter | WT | MFN2 KO |
|---|---|---|
| Growth rate (μm/s) | 0.05 ± 0.02 | 0.13 ± 0. 006^{a} |
| Shrinkage rate (μm/s) | 0.07 ± 0.004 | 0.12 ± 0.01^{a} |
| Catastrophe freq. (s−1) | 0.06 ± 0.006 | 0.06 ± 0.004 |
| Rescue freq. (s−1) | 0.08 ± 0.006 | 0.08 ± 0.006 |
| % Growth | 35.5 ± 1.92 | 44.15 ± 0.95^{b} |
| % Shrinkage | 34.05 ± 0.87 | 36.48 ± 1.27^{a} |
| % Pause | 29.58 ± 0.62 | 20.1 ± 1.45 |
| MT lifetime (s) | 58.25 ± 2.14 | 60.5 ± 1.73 |
| MT dynamicity (μm/min) | 5.96 ± 0.42 | 10.43 ± 0.25^{b} |
| Number of MTs | 20 | 20 |

^{a}p < 0.05;
^{b}p < 0.001 by Student's t-test

TABLE 3
HDAC inhibition normalizes MT dynamics in MFN2 KO MEFs

| | | | | MFN2 KO + |
|---|---|---|---|---|
| Parameter | WT | MFN2 KO | WT + TSA | TSA |
| Growth rate (μm/s) | 0.06 ± 0.01 | 0.11 ± 0.008^{a,x} | 0.04 ± 0.007 | 0.08 ± 0.005^{a,y} |
| Shrinkage rate (μm/s) | 0.08 ± 0.004 | 0.11 ± 0.009^{a,x} | 0.07 ± 0.008 | 0.08 ± 0.005^{a,y} |
| Catastrophe frequency | 0.06 ± 0.006 | 0.06 ± 0.006 | 0.05 ± 0.004 | 0.05 ± 0.006 |
| (s−1) | | | | |
| Rescue freq. (s−1) | 0.08 ± 0.006 | 0.08 ± 0.006 | 0.07 ± 0.006 | 0.09 ± 0.004 |
| % Growth | 39.5 ± 1.32 | 49.95 ± 0.95^{b,x} | 20.04 ± 1.28^{c,x} | 22.4 ± 0.90^{c,y} |
| % Shrinkage | 32.05 ± 0.87 | 37.68 ± 1.77^{a,x} | 18.25 ± 1.37^{c,x} | 16.05 ± 1.42^{c,y} |
| % Pause | 20.68 ± 0.71 | 17.1 ± 1.37 | 63.5 ± 0.64^{c,x} | 62.13 ± 1.68^{c,y} |
| MT lifetime (s) | 60.25 ± 2.05 | 61.5 ± 1.93 | 56.5 ± 3.66 | 60.25 ± 1.43 |
| MT dynamicity | 6.6 ± 0.64 | 11.63 ± 0.43^{b,x} | 4.17 ± 0.44^{a,x} | 4.858 ± 0.46^{c,y} |
| (μm/min) | | | | |
| Number of MTs | 22 | 22 | 24 | 24 |

^{a}p < 0.05;
^{b}p < 0.001;
^{c}p << 0.001 by 2-way ANOVA with Dunnett's multiple comparison
^{x}statistical comparison with WT control;
^{y}statistical comparison with MFN2 KO control

### 6.3. Example 3. Tubulin Acetylation is Required for MFN2-Dependent Regulation of Mitochondrial Motility but not for Mitochondrial Fusion or Functional Tethering to the ER

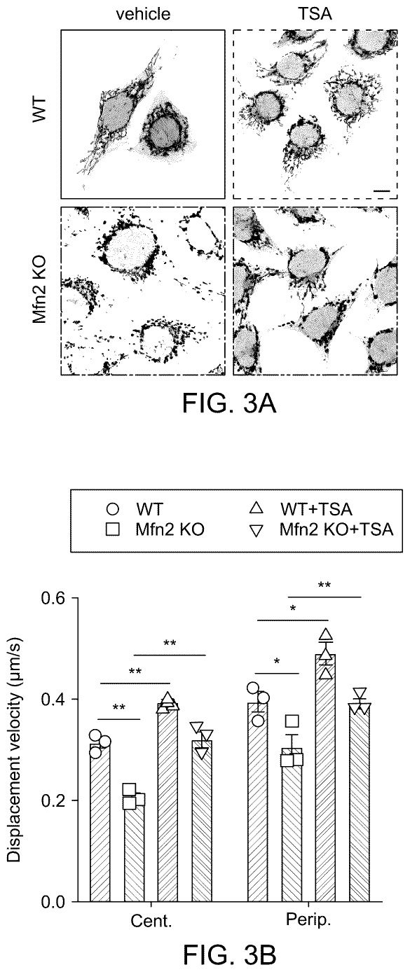

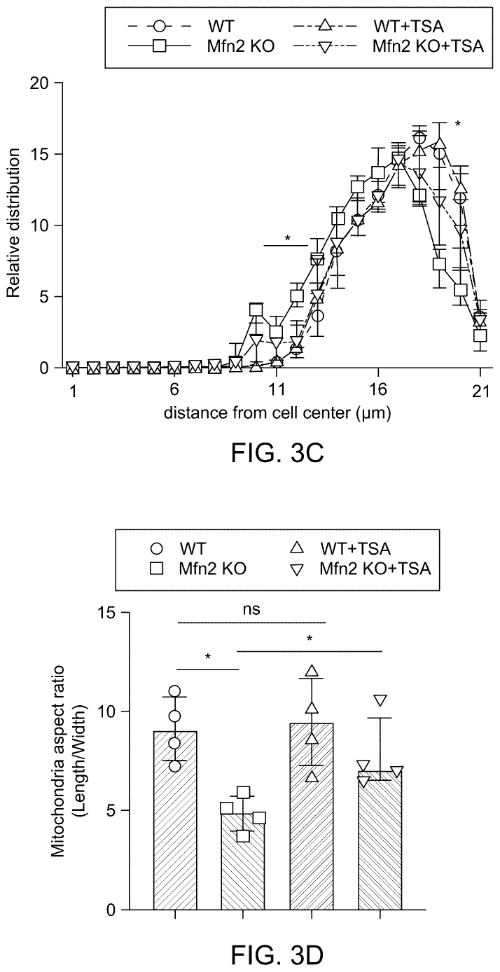

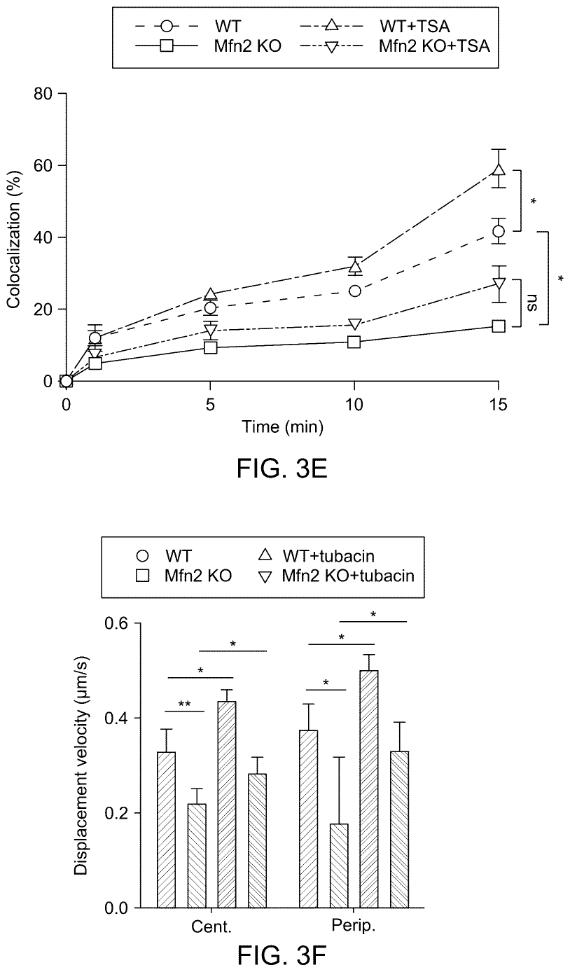

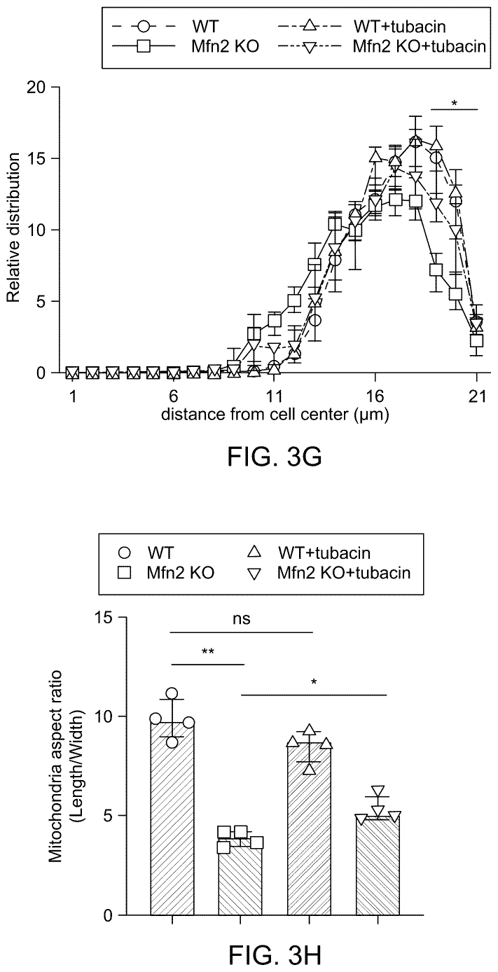

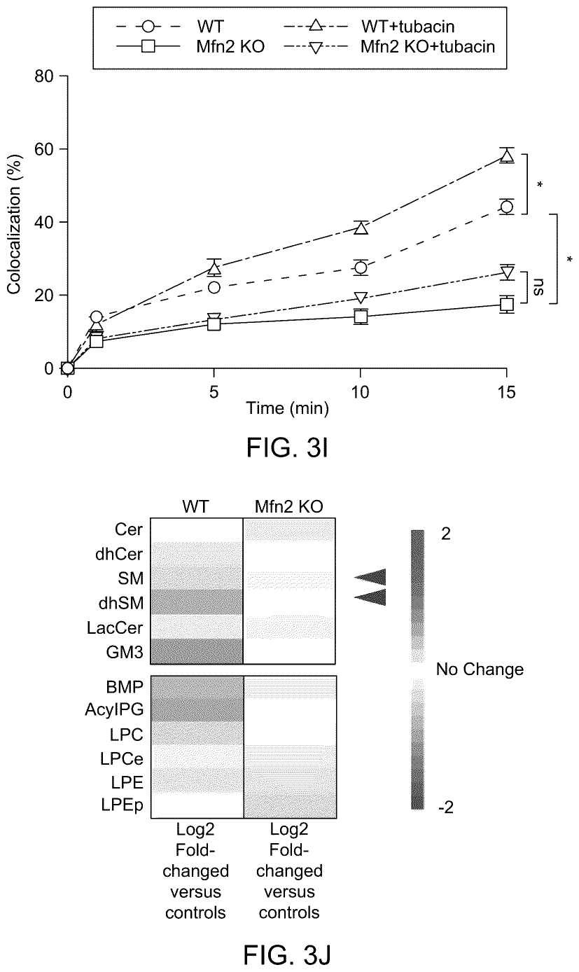

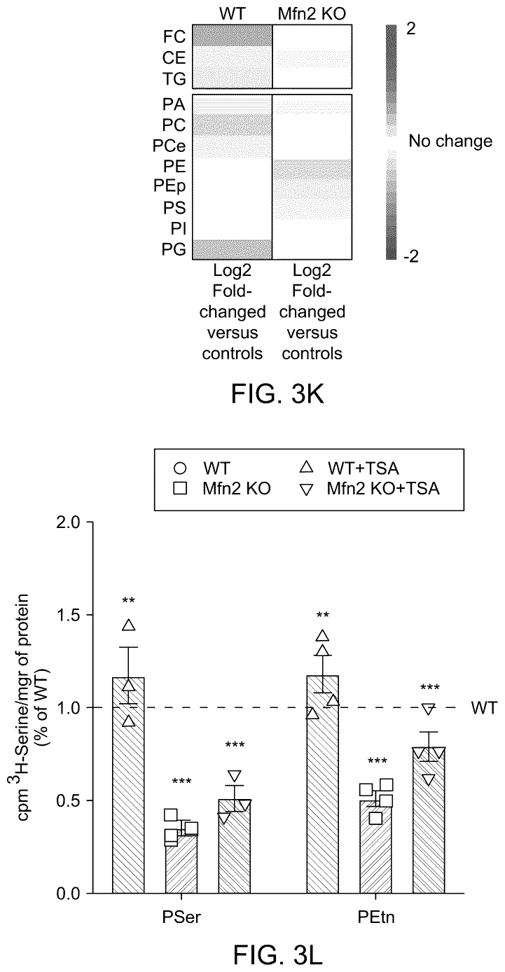

It was observed that the co-localization of mitochondria with MTs was reduced in MFN2 KO cells but restored when MFN2 KO cells were treated with TSA (FIGS. 2E and 2F). No acetylated lys 105 miro1 was detected in either WT or MFN2 KO MEFs and total miro1 appeared unchanged in MFN2 KO cells, indicating that loss of acetylated or total miro1 did not underlie the defect in mitochondrial tethering to MTs (FIGS. 2G and 2H) It was determined whether increasing acetylated tubulin levels by TSA reestablished regular mitochondria dynamics and/or mitochondrial associated ER-membrane (MAM) function, mitochondrial features affected by loss of MFN2 expression (FIGS. 3A-3L). It was observed that TSA normalized both central and peripheral mitochondrial displacement velocity as well as mitochondria distribution in MFN2 KO cells (FIGS. 3A and 3C). However, while mitochondria elongated morphology was partially reestablished in MFN2 KO cells treated with TSA, the HDAC inhibitor completely failed to recover mitochondria fusion, an activity significantly compromised in cells deprived of MFN2 (FIGS. 3D and 3E). Identical results were obtained using tubacin, a potent and selective HDAC6 inhibitor (FIGS. 3F-3I).

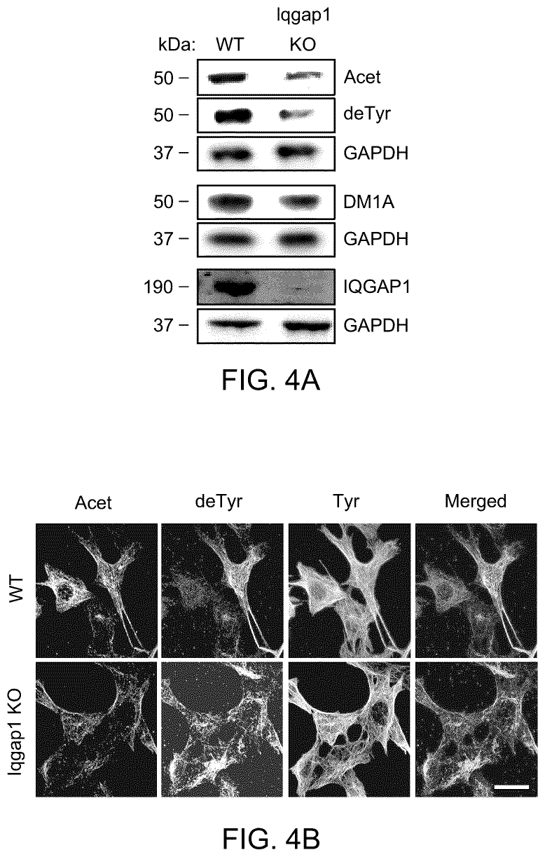

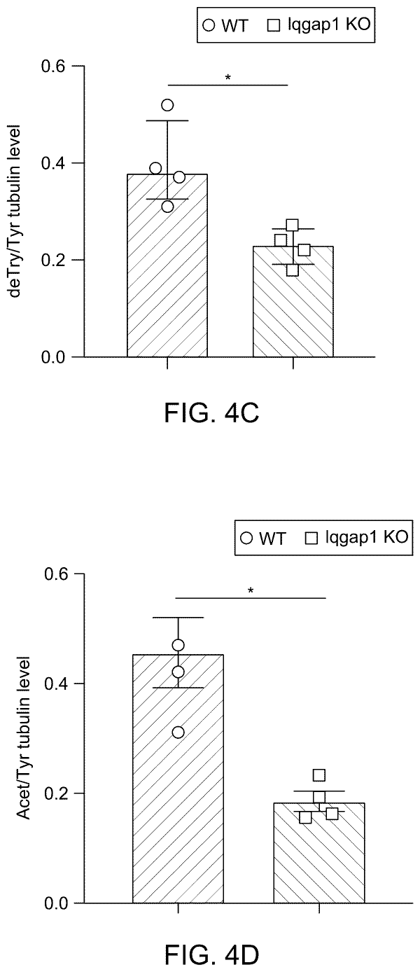

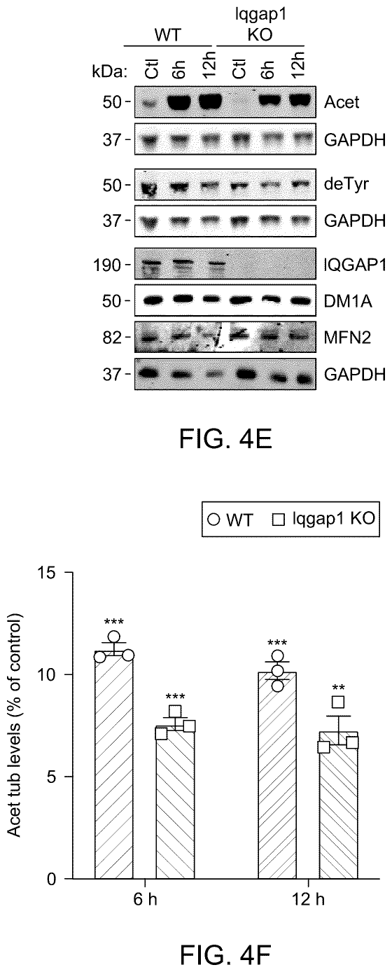

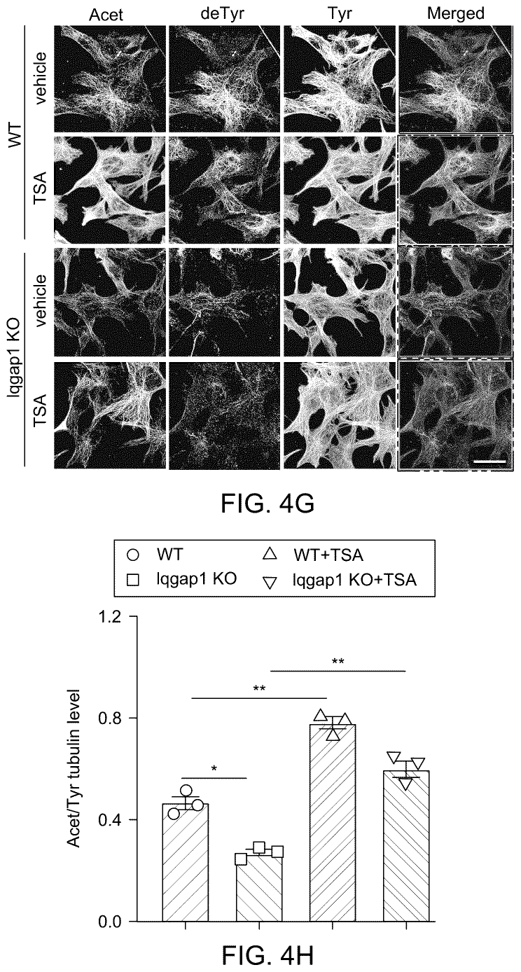

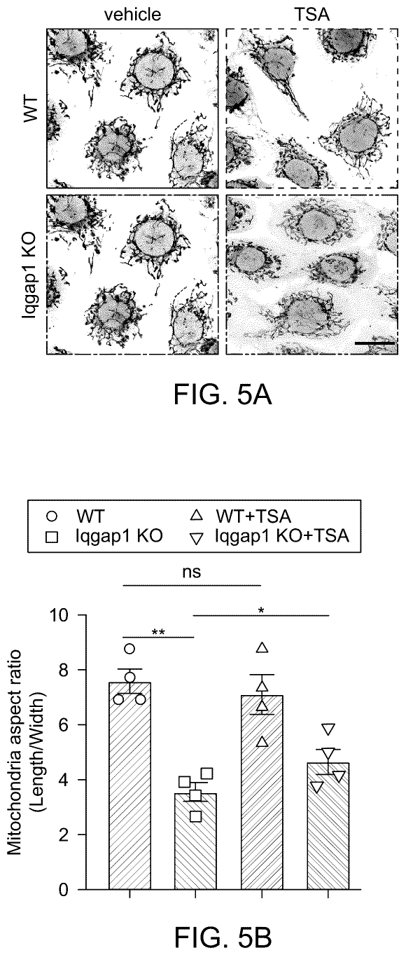

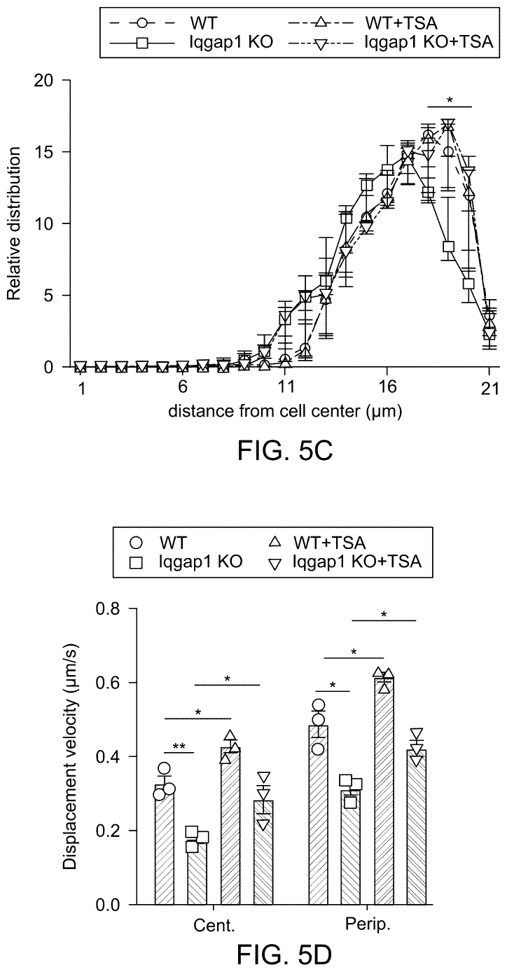

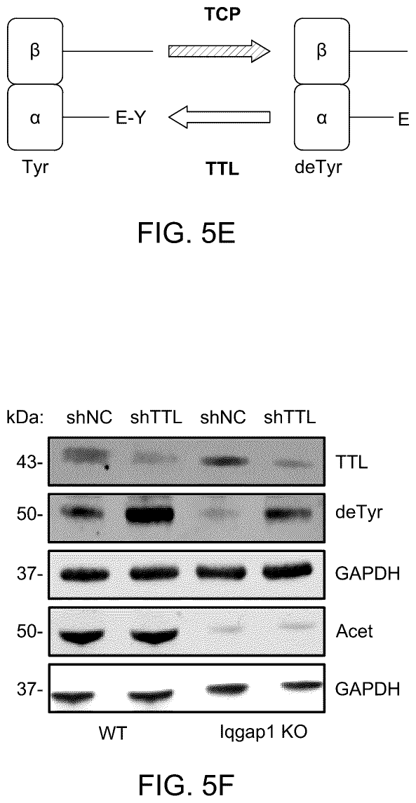

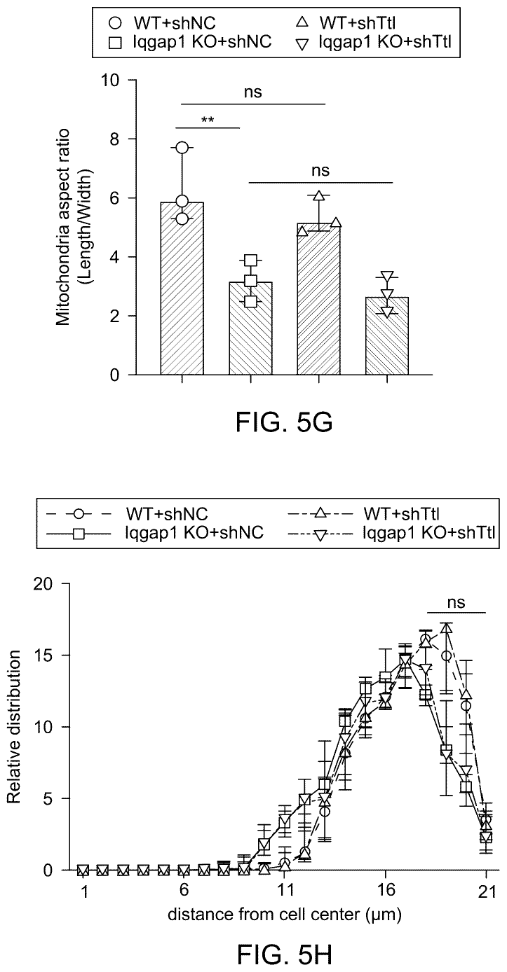

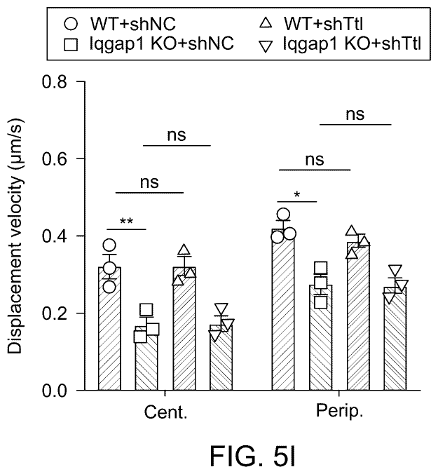

The dependence of the rescue of mitochondrial dynamics on acetylated tubulin or a general gain in MT stability resulting from tubulin acetylation was assessed. This was done using Iqgap1 KO MEFs, a cell line with normal MFN2 levels but naturally deprived of detyrosinated and acetylated MTs, two independent subsets of stable MTs (FIGS. 4A-4H). By analogy with MFN2 KO cells, it was determined that loss of IQGAP1 also resulted in defective MT and mitochondrial dynamics (FIGS. 5A-5I, and Table 4), consistent with a role for modified MTs in regulating mitochondria homeostasis. However, normalizing detyrosinated tubulin levels by tubulin tyrosine ligase (TTL) silencing did not ameliorate mitochondrial dynamics to the extent of TSA treatment (FIGS. 5E-5I), suggesting that rescue of mitochondria dynamics was dependent on the selective increase in acetylated tubulin rather than a general gain in MT stability (FIGS. 4A-4H, FIGS. 5A-5I, and Table 4).

TABLE 4
HDAC inhibition normalizes MT dynamics in MFN2 KO MEFs

| Parameter | WT | Iqgap1 KO | WT + TSA | Iqgap1 KO + TSA |
|---|---|---|---|---|
| Growth rate (μm/s) | 0.06 ± 0.008 | 0.13 ± 0.004^{b,x} | 0.04 ± 0.004 | 0.095 ± 0.006^{a,y} |
| Shrinkage rate (μm/s) | 0.04 ± 0.010 | 0.12 ± 0.006^{b,x} | 0.03 ± 0.004 | 0.09 ± 0.004 |
| Catastrophe frequency | 0.07 ± 0.004 | 0.06 ± 0.007 | 0.06 ± 0.003 | 0.06 ± 0.004 |
| (s−1) | | | | |
| Rescue freq. (s−1) | 0.07 ± 0.006 | 0.08 ± 0.010 | 0.07 ± 0.007 | 0.08 ± 0.006 |
| % Growth | 45.5 ± 2.021 | 45 ± 1.354 | 23.75 ± 0.85^{b,x} | 25.25 ± 1.54^{b,y} |
| % Shrinkage | 31.5 ± 2.63 | 30.18 ± 2.254 | 27.25 ± 3.19 | 22.5 ± 0.86^{b,y} |
| % Pause | 22.5 ± 1.041 | 24.75 ± 1.652 | 50 ± 2.48^{b,x} | 51.75 ± 1.10^{b,y} |
| MT lifetime (s) | 47.5 ± 2.533 | 51 ± 1.472 | 49.5 ± 2.32 | 51.25 ± 1.79 |
| MT dynamicity | 5.22 ± 0.317 | 8.17 ± 0.606^{a,x} | 3.32 ± 0.24^{a,x} | 3.77 ± 0.33^{b,y} |
| (μm/min) | | | | |
| Number of MTs | 20 | 21 | 25 | 25 |

^{a}p < 0.001;
^{b}p << 0.001 by 2-way ANOVA with Dunnett's multiple comparison
^{x}statistical comparison with WT control;
^{y}statistical comparison with Iqgap1 KO control

Next, the effects of restoring acetylated tubulin levels on loss of MAM function was determined by analyzing the synthesis and transfer of phospholipid between ER and mitochondria, a known proxy of MAM activity, as well as changes in lipid classes by lipidomics analysis in MFN2 KO cells [. MAM is a transient specialized subdomain of the ER with the characteristics of a lipid raft. The temporary formation of MAM domains in the ER regulates several metabolic pathways, including lipid and Ca²⁺ homeostasis and mitochondrial activity. Alterations in the formation of MAM domains have been reported to induce significant changes in lipid metabolism in several pathologies including neurodegenerative disease. In particular, defects in MAM activity have significant detrimental effects on the regulation of cholesterol and its esterification into cholesteryl esters. Equally important, defects in MAM impair the regulation of sphingomyelin (SM) turnover and its hydrolysis into ceramide species.

MFN2KO cells were found to display significant increases in sphingomyelin and cholesterol with concomitant decreases in cholesteryl esters and ceramide levels (FIGS. 3 and 3K, Table 5) and that these changes could be rescued by TSA (FIGS. 3J and 3K). Conversely, TSA failed to normalize MAM-dependent phospholipid synthesis, measured by incorporation of radiolabeled ³H-Ser into newly synthesized ³H-PtdSer (PS) and (H)³H-PtdEtn (PE) (FIG. 3L).

TABLE 5
List of internal standards used to calculate the
concentrations of corresponding lipid classes

| Internal Standard | Corresponding Lipid Class | Concentration (μg/μl) |
|---|---|---|
| IS AcylPG 14:0-28:0 | Acyl PG, NAPE, NAPS | 0.046799614 |
| IS BMP 28:0 | BMP | 0.015298133 |
| IS CE C17 | CE | 78.59098931 |
| IS Cer C17:0 | Cer, dhCer | 0.758320608 |
| IS Chol d7 b | Free Cholesterol | 63.78791732 |
| IS DG 4ME | diacylglycerols | 0.640874053 |
| IS dhSM d18:0/12:0 | dihydrosphingomyelins | 2.579623778 |
| IS DMPC | AC | 12.34642208 |
| IS GalCer d18:1/12:0 | MhCer | 1.039897431 |
| IS LacCer d18:1/12:0 | LacCer | 0.259594347 |
| IS LPC 13:0 | LPC | 12.34642208 |
| IS LPE 14:0 | LPE | 0.098349468 |
| IS LPI 13:0 | LPI | 0.07642123 |
| IS MG C17 | MG | 0.242952978 |
| IS PA 28:0 | PA | 0.068072997 |
| IS PC 28:0 | PC | 12.34642208 |
| IS PE 25:0 | PE | 8.839285714 |
| IS PG 12:0/13:0 | PG | 0.446428571 |
| IS PI 12:0/13:0 | PI | 2.232142857 |
| IS PS 28:0 | PS | 11.92531331 |
| IS SM d18:1/12:0 | SM | 13.39285714 |
| IS Sulf d18:1/12:0 | Sulf | 0.225924621 |
| IS TG 50:0 d5 | TG | 0.498018035 |

Altogether, these results demonstrate a previously unrecognized role for MFN2 in the regulation of α-tubulin acetylation and suggest that this activity is important for MFN2-dependent control of mitochondria motility and lipid-raft MAM composition, but not for MFN2-dependent mitochondrial fusion or functional mitochondrial/ER tethering. Furthermore, these results in Iqgap1 KO cells support the notion that acetylated tubulin is a modulator of mitochondria dynamics per se and suggest that the machinery controlling mitochondria motility likely regulate the α-tubulin acetylation cycle at sites of mitochondria contacts with MTs.

### 6.4. Example 4. MFN2 Regulates α-Tubulin Acetylation by Recruiting ATAT1 at Sites of Mitochondrial Contacts with MTs

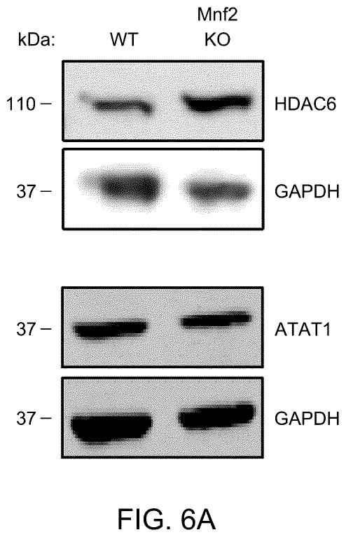

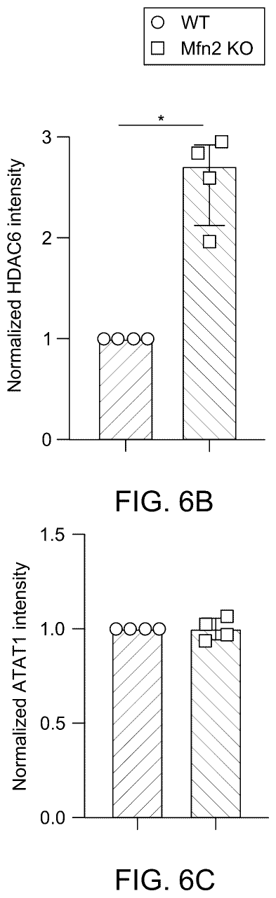

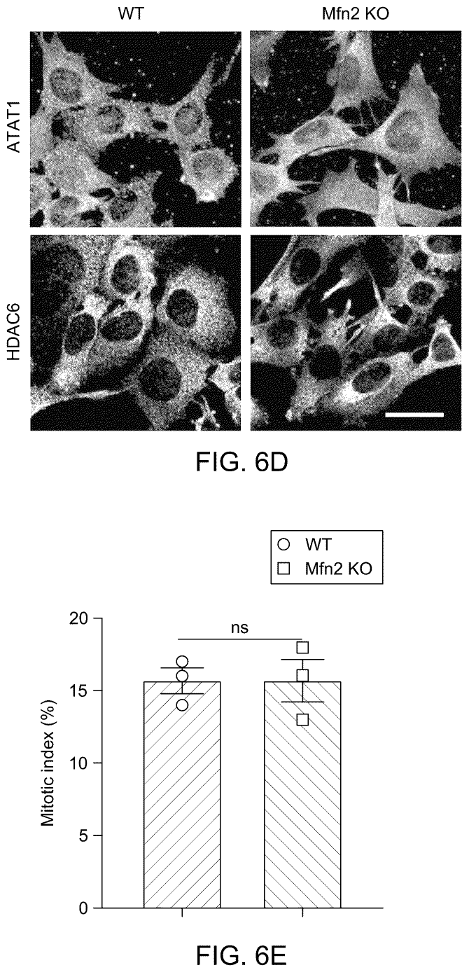

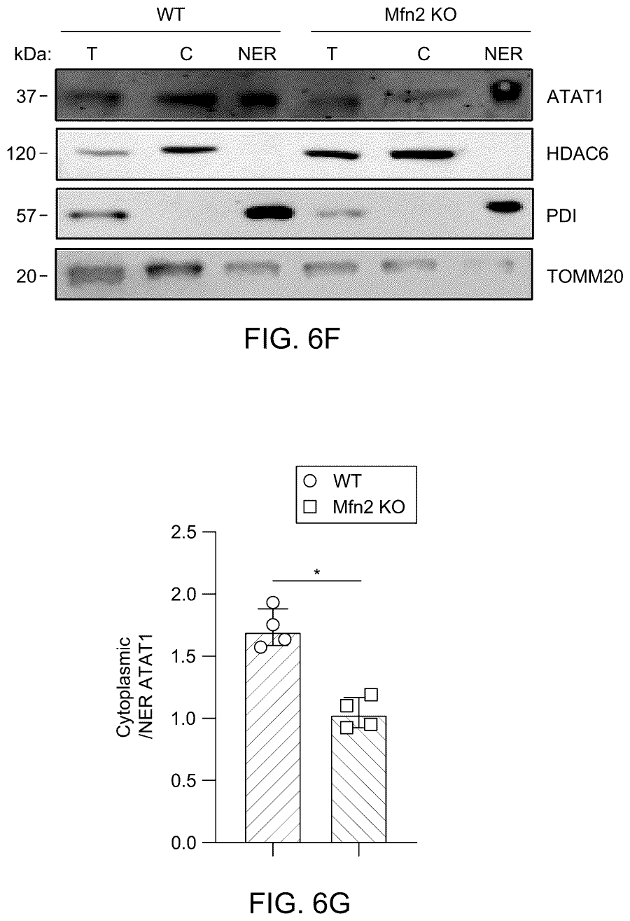

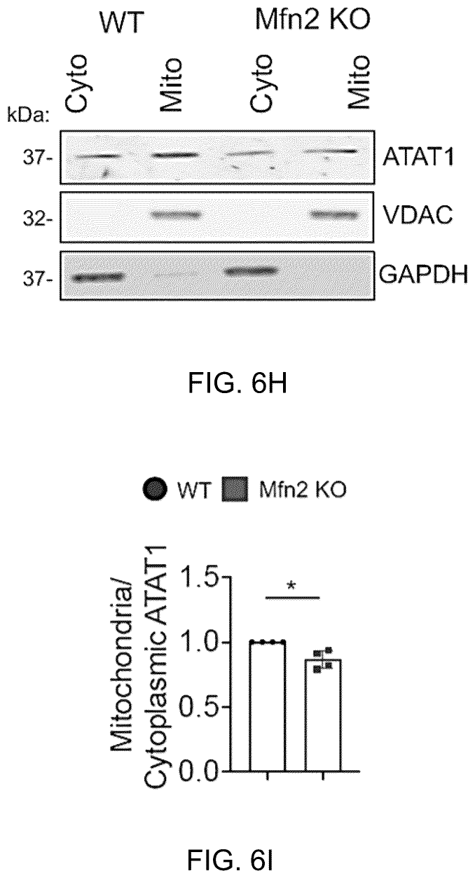

The mechanisms underlying MFN2 regulation of acetylated α-tubulin were investigated by measuring levels and localization of ATAT1 and HDAC6 in MFN2 KO cells. HDAC6 expression was three-fold higher in these cells, in contrast to ATAT1 levels, which remained unaffected (FIGS. 6A-6C). Loss of MFN2 expression did not affect the percentage of cells in mitosis either (FIGS. 6D and 6E). However, when intracellular membranes were subjected to crude fractionation to isolate the cytosolic from the nuclear and ER fractions, unlike HDAC6 which remained mostly cytosolic, ATAT1 appeared in the cytosolic and in the nuclear/ER portion in WT cells but re-distributed more prominently to the nuclear/ER fraction in MFN2 KO cells (FIGS. 6F and 6G). Accordingly, localization of endogenous ATAT1 with the mitochondria enriched fraction appeared to be decreased in MFN2 KO cells compared to WT cells (FIGS. 6H and 6I).

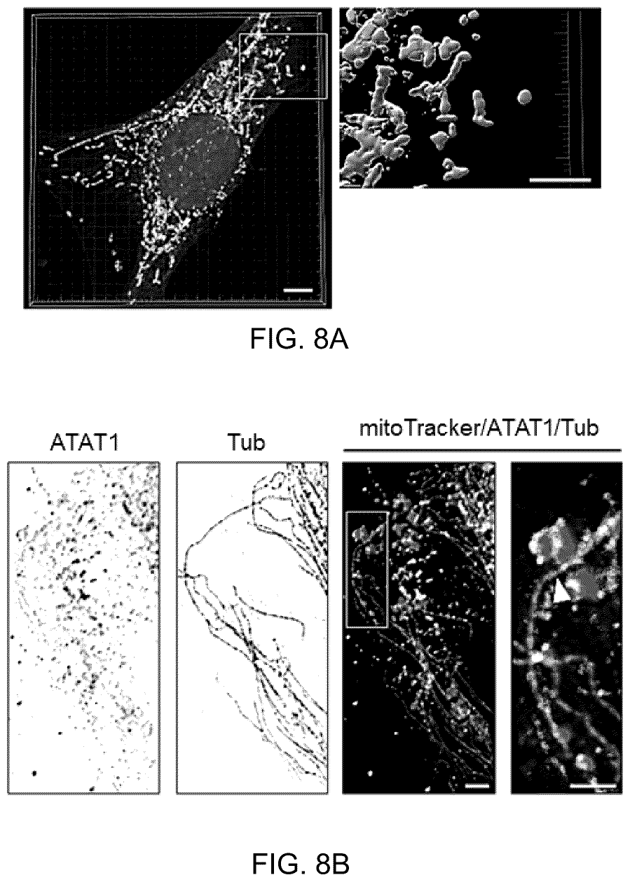

It is hypothesized that MFN2 can negatively regulate ATAT1 association with the ER by localizing ATAT1 to mitochondria outer membranes, and that this localization can facilitate the access of ATAT1 to openings of the MT lattice at sites of mitochondria contacts with MTs. High resolution confocal microscopy of endogenous proteins revealed punctuate localization of ATAT1 to mitochondria membranes or MFN2, and this co-localization was lost in cells deprived of MFN2 expression (FIGS. 7A-7E). Importantly, almost 80% of mitochondrially localized ATAT1 (˜60% of total ATAT1) also localized with endogenous MFN2, and ATAT1 decorated mitochondria were often observed to be in contact with MTs that appeared to be nicked (FIGS. 7F and 7H, 8A and 8B). Localization of ATAT1 to mitochondria was likely to be dependent on the association of MFN2 with an ATAT1 N-terminal fragment (1-242) inclusive of its catalytic domain, as demonstrated by the in situ validation of this interaction using the proximity ligation assay (FIGS. 7I and 7J), co-immunoprecipitation from cells overexpressing either full length Myc-MFN2/Flag-ATAT1 or either Myc-MFN2 or FLAG-ATAT1 alone (FIGS. 7K and 8C), and conventional pull down analyses from whole cell lysates using full length or C-terminally truncated versions of ATAT1 (FIGS. 7K-7M). Lack of binding to TOMM20 or COX4, the subunit 4 of the inner mitochondrial enzyme cytochrome c oxidase, confirmed that the association with MFN2 was not due to non-specific stickiness of ATAT1 to mitochondria (FIG. 8C). Altogether, these data demonstrate that ATAT1 associates with mitochondria and that this localization is dependent on the binding of the catalytic domain of ATAT1 with MFN2.

### 6.5. Example 5. Loss of Acetylated Tubulin in CMT2A Disease

Most MFN2 CMT mutations are missense, and all produce a dominant inheritance pattern, suggesting that mutations in MFN2 lead to either a gain of function or haploinsufficiency. Furthermore, recent work supports the notion that restoring MFN1:MFN2 balance by increasing levels of its homologous protein MIFN1 is a potential therapeutic approach for CMT2A. The reason for this compensation is unclear, although both MFN2 and MFN1 have been implicated in mitochondria fusion. To determine the involvement of MFN2-dependent regulation of tubulin acetylation in CMT2A disease it was investigated whether: 1) mutations in MFN2 affect the interaction with ATAT1 and/or fail to restore normal acetylated tubulin levels in MFN2 KO cells; 2) MFN1 compensates for loss of MFN2 by restoring tubulin acetylation in MFN2 KO cells; 3) loss of acetylated tubulin by MFN2 depletion is conserved in sensory neurons and sufficient to induce axonal fragmentation, a phenotype associated to axonal forms of CMT disease including CMT2A.

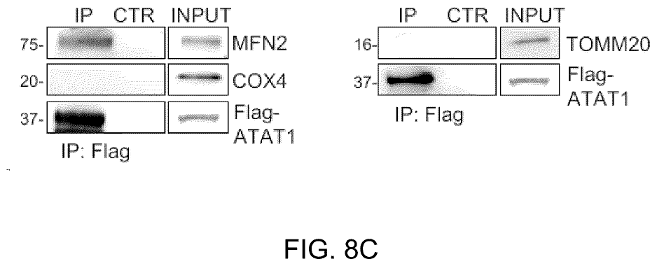

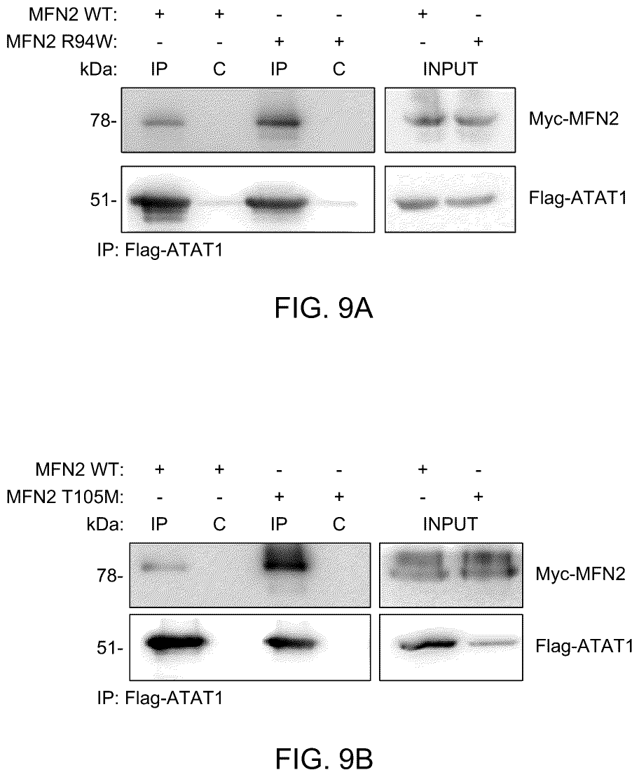

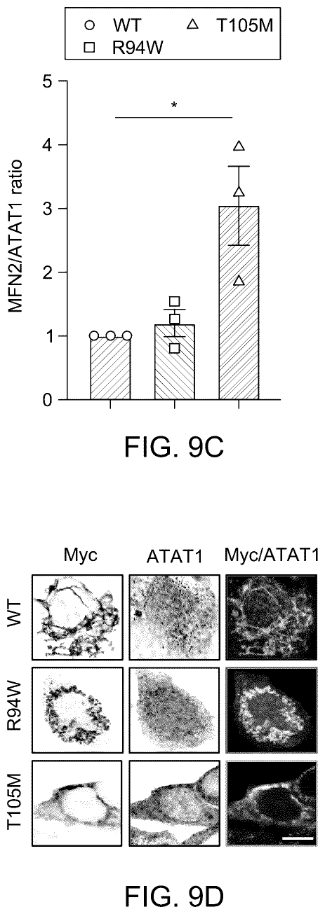

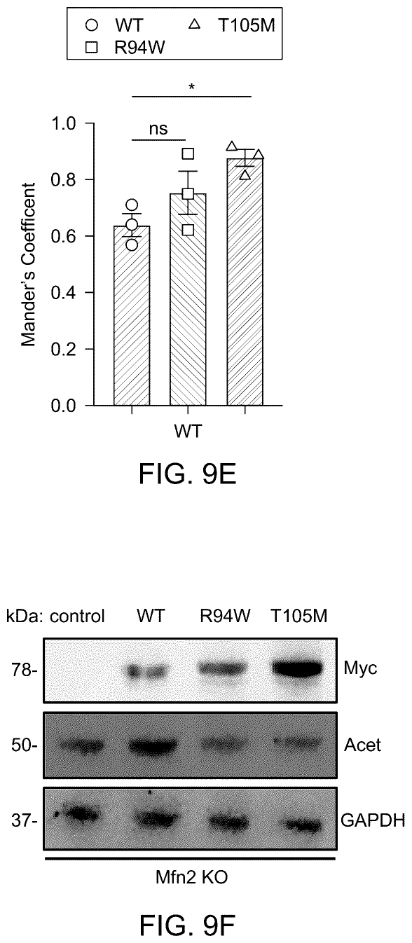

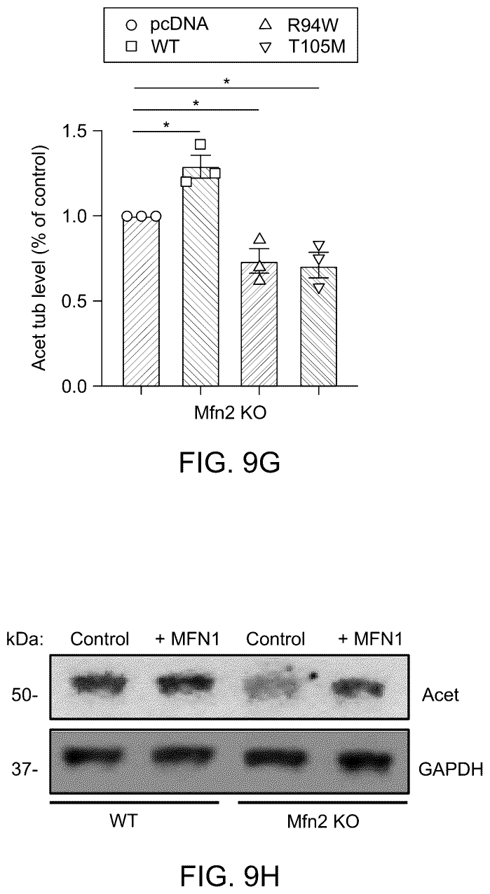

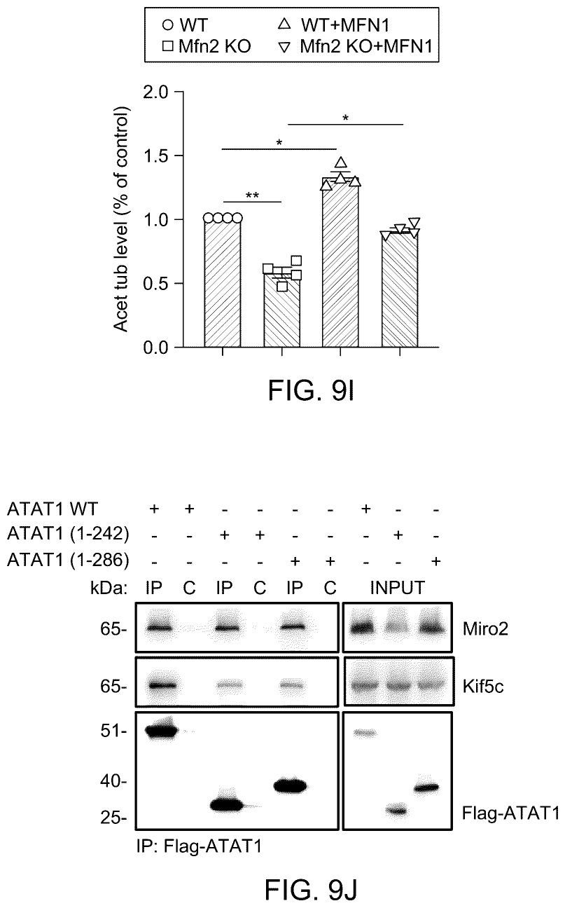

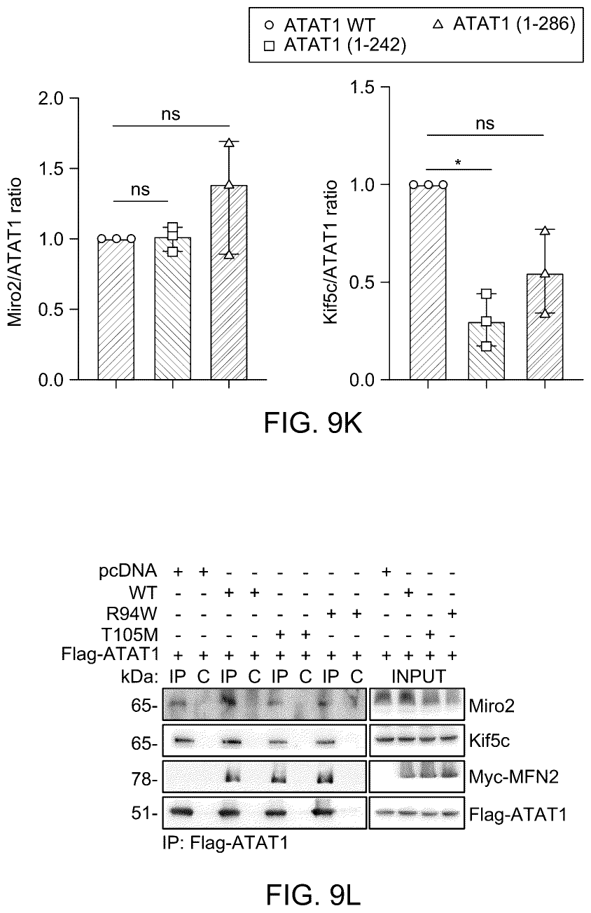

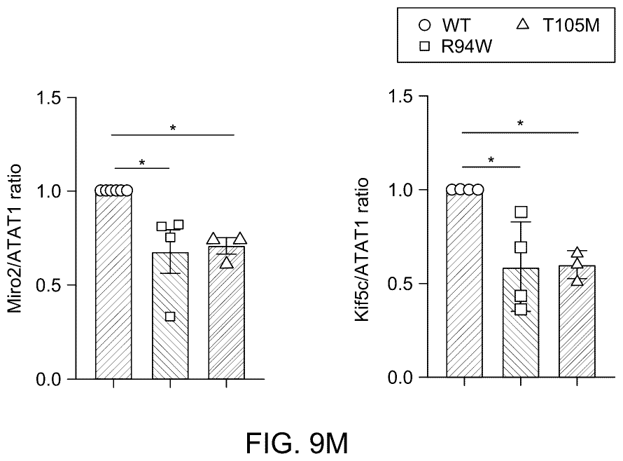

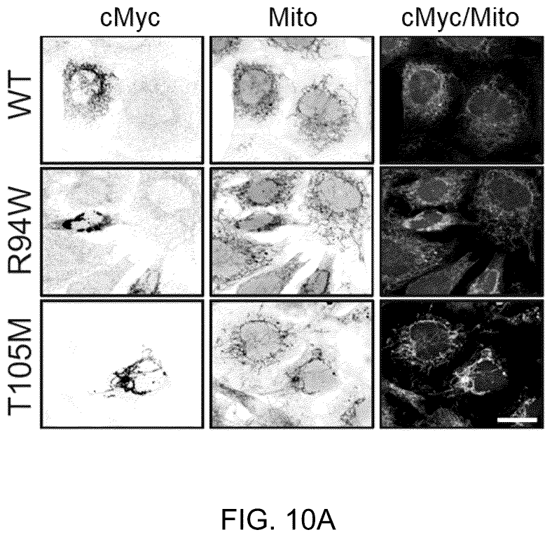

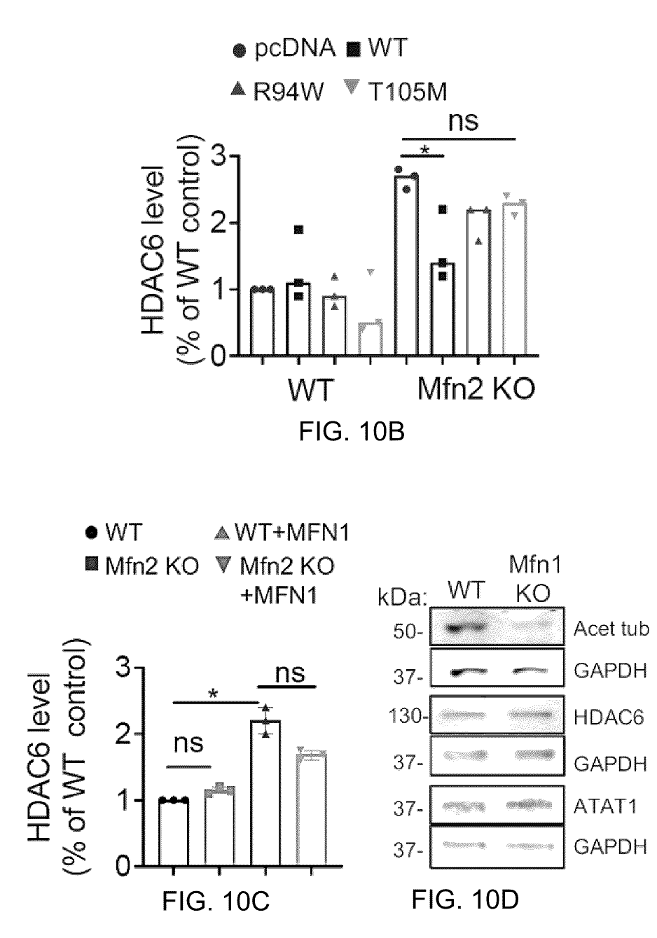

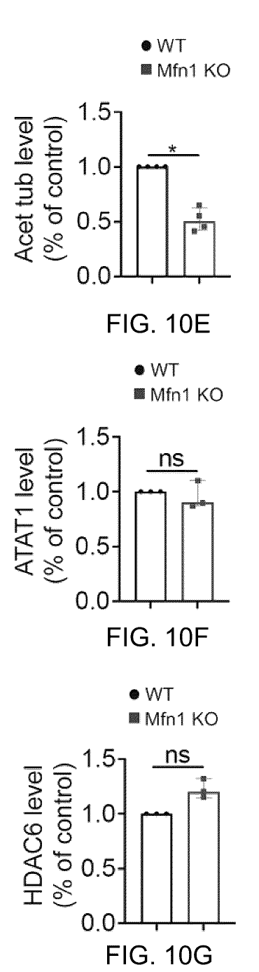

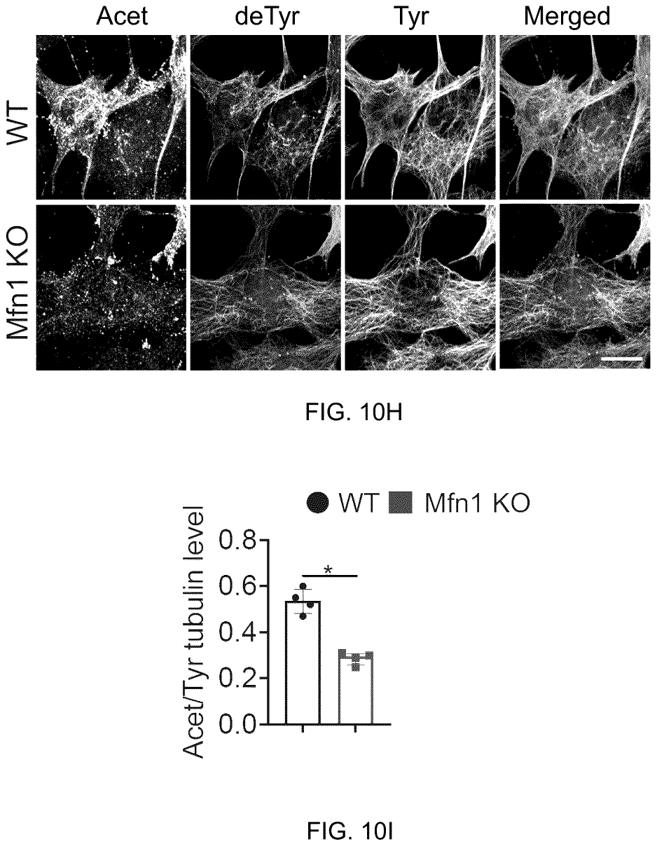

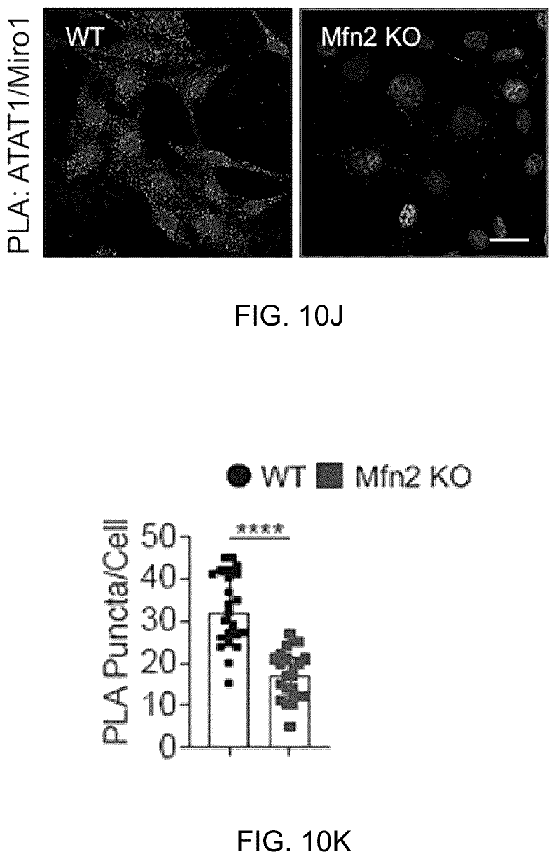

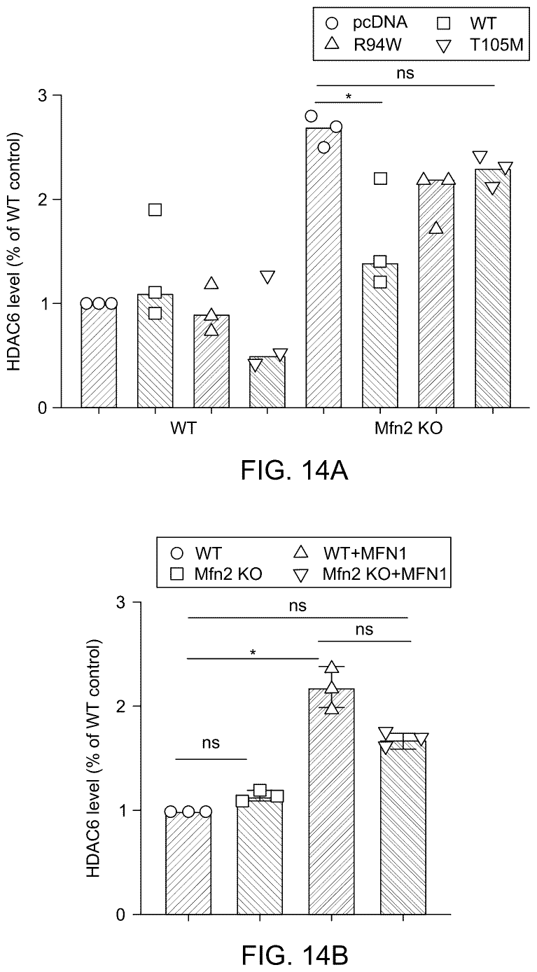

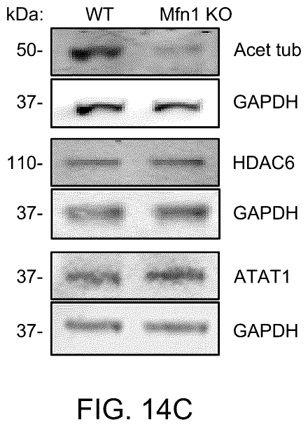

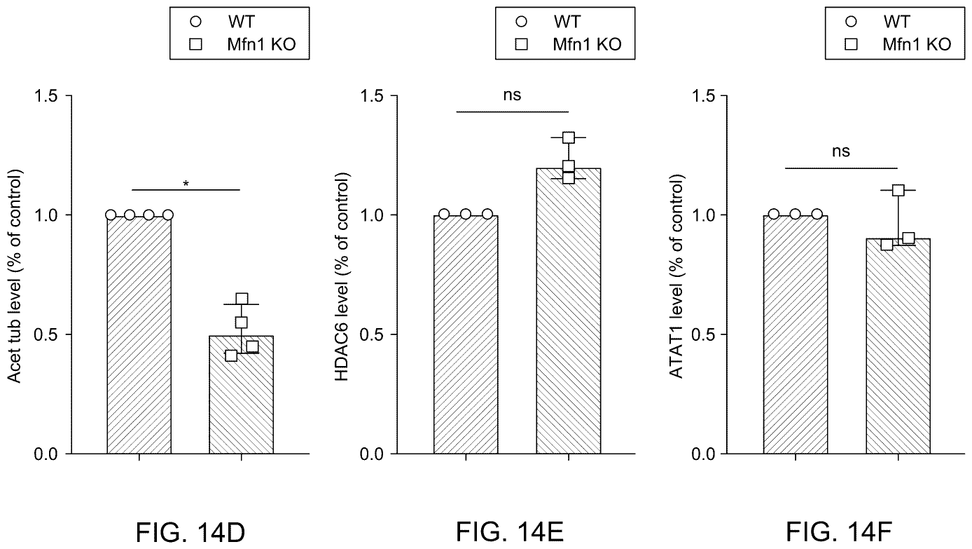

It was determined that MFN2 R94W and T105M, two of the most common N-terminal CMT mutations in MFN2 that do not lose their association with mitochondria (FIG. 10A), bound to ATAT1 with higher affinity than WT MFN2 (FIGS. 9A-9C) and endogenous ATAT1 co-localized better with mutant MFN2 (although only with T105M to a significant degree) than WT MFN2 (FIG. 9D-9E). However, both mutations failed to rescue normal acetylated tubulin levels when expressed in MFN2 KO cells (FIGS. 9F and 9G). This result was in stark contrast with ectopic expression of MFN1, which was fully able to compensate for loss of MFN2 on acetylated tubulin levels in MFN2 KO cells (FIG. 9H-9I). The effects of overexpressing WT and mutant MFN2 or WT MIFN1 on HDAC6 protein levels in MFN2 KO cells were examined and found that while only WT MFN2 normalized it, WT MFN1 expression had no significant impact (FIGS. 10B, 10C, 14A and 14B). In addition, complete loss of MIFN1 expression in MFN1 KO MEFs caused ˜50% reduction in tubulin acetylation without altering either ATAT1 or HDAC6 protein levels (FIGS. 10D-10I and 14C-14F), implying that modulation of HDAC6 was not the only mechanism underlying MFN-mediated regulation of tubulin acetylation.

A complex between miro/Milton (TRAK) and MFN2 has been previously shown, and miro has been implicated in regulating MFN2-dependent mitochondrial fusion in response to mitochondrial Ca²⁺ concentration. The interaction of ATAT1 with miro and/or kinesin heavy chain (Kif5c) were tested and the potential effects of mutant MFN2 on the formation of these complexes determined. Using proximity ligation assays, an association between endogenous ATAT1 and Miro1 in WT MEFs detected, which was significantly disrupted in MFN2 KO MEFs (FIGS. 10J and 10K). It was determined that ectopic ATAT1 co-immunoprecipitated with both endogenous miro2 and kif5c and that ectopic expression of mutant MFN2 R94W or T105M significantly lowered the affinity of these bindings (FIGS. 9J-9M). Taken together, these data demonstrate that regulation of acetylated tubulin is an activity shared by MFN1 and that loss of acetylated tubulin likely play a primary role in CMT2A via the sequestering effect of MFN2 mutations on ATAT1 from miro and kif5c binding.

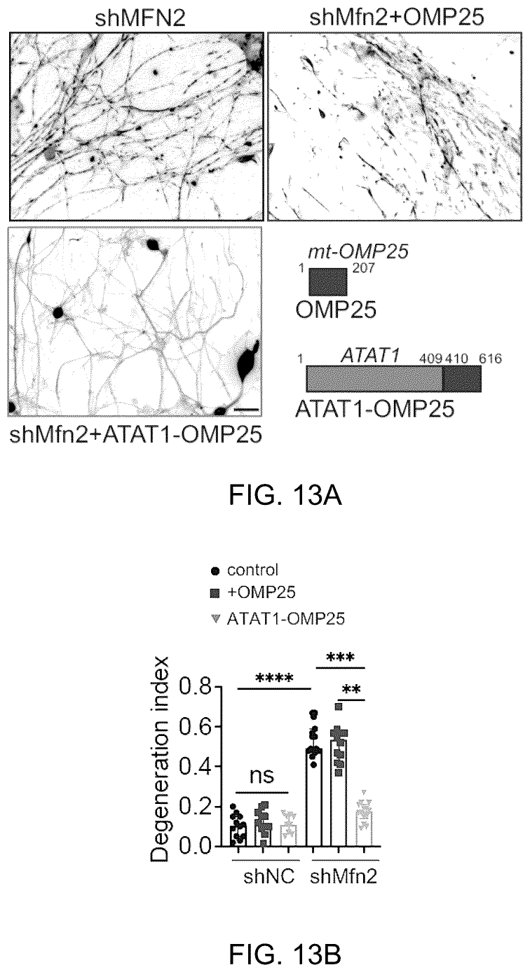

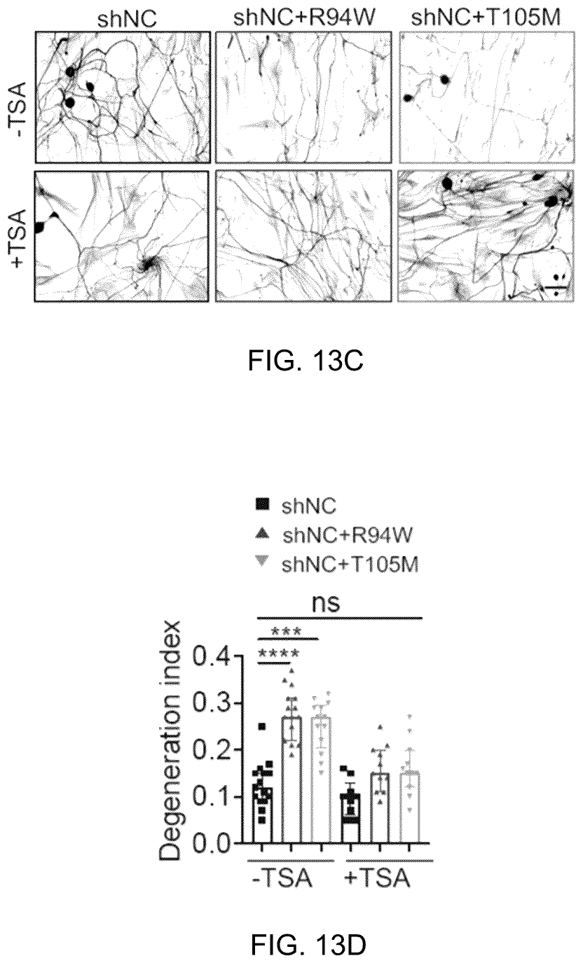

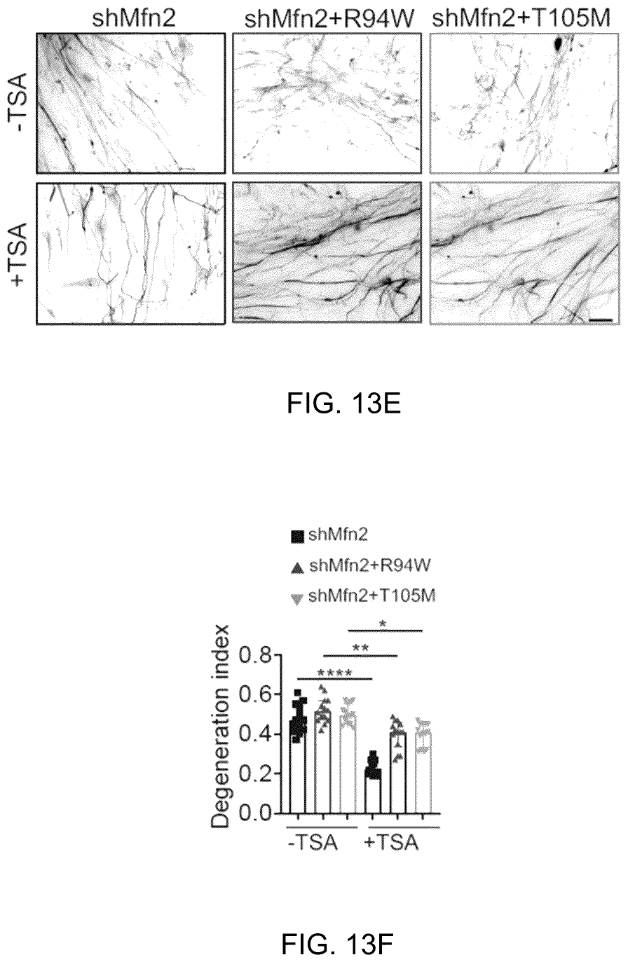

These observations became particularly meaningful when the consequences of loss of MFN2 in sensory neurons and the effects of HDAC6 inhibition on these phenotypes were tested. By analogy with MFN2 KO cells, silencing of MFN2 expression reduced acetylated tubulin levels both in adult mouse DRG neurons grown in culture and in cell bodies of somatosensory neurons of third instar stage Drosophila larvae (FIG. 11A-11E). Similar to MFN2 KO MEFs was the observed localization of endogenous ATAT1 and MFN2 in DRG neurons (FIGS. 12A-12C) and significant reduction in the extent of ATAT1 localization to mitochondrial membranes in neurons silenced for MFN2 expression in both proximal and distal portion of the axon (FIGS. 12D-12I). Importantly, cultured sensory neurons deprived of MFN2 acquired a dying-back degeneration phenotype starting from distal regions of the axon, as indicated by the appearance of retraction bulbs at the onset of axonal fragmentation (FIGS. 11IF-11Ht). It was observed that loss of acetylated tubulin preceded axonal degeneration in DRG neurons deprived of MFN2 for shorter times (FIGS. 12J-12N) and that HDAC6 inhibition, which significantly rescued normal acetylated tubulin levels in MFN2 KD neurons, prevented both retraction bulb formation and axonal degeneration in neurons deprived of MFN2, while having only negligible effects on WT controls (FIGS. 11I-11L). Notably, a role for any HDAC6 substrate other than tubulin was ruled out by inducing rescue in MFN2 depleted DRG neurons with expression of ATAT1 localized to mitochondrial outer membranes by fusion with the OMP25 targeting sequence (FIGS. 13A-13B).53 In addition, while neither MFN2 CMT2A mutants could compensate for MFN2 loss on axonal degeneration, both mutants were sufficient to induce about 2.5-fold increase in axonal degeneration if expressed in control DRG neurons in the absence of HDAC6 inhibition (FIGS. 13C-13F and Tables 6 and 7).

TABLE 6
Sequences

| Description | | SEQ |
|---|---|---|
| (See FIGS. | | ID |
| 13A-13F) | Sequence | NO.: |
| GenBank: | GAGAGCCCAGCCTTCTCCCTGGCTCCTATTTAAGCATCA | 1 |
| AF107295.1 | GGCAAAGACCGTGGGTCCCAGCAACAGCAGTGAGGAG | |
| Rattus | GCACTTGGCACTTCGGGCAGGTGGTAGCCGCTGTGCCT | |
| norvegicus | GGCACCTTGGCATCCTGCCTGGGCTGCACGTGGCCTGG | |
| outer membrane | CACCCATACAGCTCCTGATTGCTGCGGCTGGAAGGAGC | |
| protein | CTGTGATTCTGCGTGAGTGTGGGGTCCTTGGGTATACAG | |
| (OMP25) | TCAGCCGAGGTAGAGGTGGGCTGGCCTAGGACTGTACA | |
| mRNA, | CTTCTCTAGGAGCTGGGCTTAACTAAAACAGAAGAGAT | |
| complete | TGCGGTTAGACATCAAGAGGAACTCAGACCCGTGGGAA | |
| cds; nuclear | AGAACCAGGACCTGAGGCAAAGGCGAGATTTGGGTGGT | |
| gene for | GGACGGGGGACAGGGAGACTCTTCACAGTCCTCACAGG | |
| mitochondrial | ACCTACACAGGGGCCTATGGTCTCAGACCTGGGATCAT | |
| product | GTTCCTGAGGGGGTATAAGAGAGAGGGGCAGGTCAGG | |
| cds 456 . . . 1076 | AAGCATAGGAAGTCAGTTGCACATGGGGGAGGGTTGTG | |
| (underlined) | GGGAAACTGTAAAAGCTATGCTGGGGAGGCGGCACGG | |
| c-terminal | AGCTTGATTCACCTTCACCTGCGCCGGGCACCCGCTGAC | |
| domain (bold) | CCCGGGTTTCCGCCCGGAGAGCAGTCAGATATGAACGG | |
| | ACGGGTGGATTATTTAGTCTCCGAGGAAGAGATCAACC | |
| | TGACCAGAGGACCCTCGGGGCTGGGCTTCAACATCGTC | |
| | GGTGGGACAGATCAACAGTATGTCTCCAATGACAGTGG | |
| | CATCTACGTCAGCCGCATCAAAGAGGATGGGGCTGCGG | |
| | CCCGGGATGGGCGGCTCCAGGAGGGTGATAAGATCCTC | |
| | TCGGTAAATGGCCAAGACCTGAAGAACCTGTTGCACCA | |
| | AGATGCCGTAGACCTCTTCCGTAATGCAGGATATGCCG | |
| | TGTCCCTGAGAGTGCAGCACAGGTTACCAGTGCAGAAT | |
| | GGACCTATAGTTCATCGAGGCGACGGAGAGCCGAGTGG | |
| | AGTTCCTGTAGCTGTGGTGCTGCTGCCAGTGTTTGCCCT | |
| | TACCCTGGTAGCAGTTTGGGCCTTCGTGAGATACCGAA | |
| | AGCAGCTCTGAGATGCCTGTTGTCTTCCAGTGTGTCCGA | |
| | TGAGCTAACTATCTCTCTCACTCACCATCTCGACATCCT | |
| | CCCCTAGTCTTCCTTCCCACATAGCCAACACATGATTTA | |
| | AAGTGACTGCTTATCACCCGAAACCTTGCTGTTCAAAAT | |
| | CTCCAAGACTTCACATTCTAATGGAAGAGTAAAGAGAT | |
| | TATTTGAAGAAAGCTGGGGGTGGGGAGAGCCTTGCTTA | |
| | GAATAAATGAGAAGTTACATATTTTACTAGAACTGCCA | |
| | ATAAAAATTCAGCTATCAGCCAAAGAGGAGAAGCTTGC | |
| | TCTTCCTGTCTCCATGGACGACACCTTTTGCTTAGCTGG | |
| | TGTGCTTTGAAGGCTAGCTGTGCTATGTGAAAGGAGGA | |
| | GCTGATTTTTTAAATACTTTTTCTTGGGAAGTATTTGTG | |
| | GCCTTTAATTTGTAACTATATACTTAGATGCCTATATTG | |
| | GACATAGGCGAATGAATTTTTTTCTTTTCTTAGAAAAGA | |
| | AAATACATATATACATTAACACACACACACACACACAC | |
| | ACACACACAGCATAAAACTGATGCCTTATGGAGAGTTA | |
| | AAGAGGTGAGAAAACTACTGGTTCTTGGATTTCTAGTG | |
| | GACAAGTTTTGGGAACTAGGGGGTCATATTCCTTTATAT | |
| | ATAATCAAAACTCATATTAAAGAATGAGTTCTGGATTG | |
| | TAAAGAGAACTTACTTTTTCCACTTGTCTGTAAGTCTTT | |
| | GTCCACAAGTTAAAAACATACACAGTCCTAAGGGCTGA | |
| | TCATAACTGAAACACTTCAAAAATTATTGGCAGAGAAG | |
| | GTATAACGGGGATGGAAAAGTTAGCTTTAAAAGAGAAT | |
| | GCTCAGTCAGTGGCATTAGGAAAATAATGACTGAAGCA | |
| | ATTAGTTGAAAACTGTAGAACAACTAGTGTTCCAGGGT | |
| | AGCTACAGTGATGTGGGAAATTGTGGCAAAGCAGTTCT | |
| | TCTCAAGTCAATTTGTTTAAAAGACTTTGGGGTGTAAAG | |
| | ATGAGTCCTGACAGTCTCTGAATGACCTTAGTGACTGTT | |
| | GTACTATGTGAAAGGATCCATTGGCATGGAGATGAGAA | |
| | GTAGGAGGGCCAAATAAACCATCAAGACAACTGAAAA | |
| | AAGATCATACCTCAGGGTGGCACTTGAACCCTCAAAGT | |
| | GCTCACCAACTTTCAGTTTCTAGTATCAGGCAAAGCCTA | |
| | AGGGAAGGGACTGCTTTCCTGGCAGGGTGGACCCCAAA | |
| | TCAGCCTGCTTCTGATAGAGTTACGATGAGAAGGCCCA | |
| | AATTGATGTAGGCTTTCTTTTTGTGTATTTTTCCCAAAGC | |
| | TATAAGCACTAGTTTTGGTTTTAATAAGGTAATGGACCA | |
| | TTTTTTCAGAAAAAGGGGAAGGAATAAGGTTTGTTTGTT | |
| | TATAGAGGGTGCAAGGAAGTTTAGTGAGCATTTCTGAG | |
| | GAAAGCCTGAGCACACAGAGTAGATTAACTATGCCAGA | |
| | GAATAGCCAGGGATATTGGGGGGTGGGGGGTAGTGGA | |
| | GCTGTCCTGTTTATGTAGCATCAAAGTTGGTTTTAGAGT | |
| | ACTGAGGATAGTTGAACCAAGCCTAATTTTTTTTTTCAC | |
| | TTTAACAAAATAATTTCCGTCTTTTCAATAAATTGGCCA | |
| | GACTATTTTGACCTAAGATTATAAAGAGTTTTCCTGACA | |
| | ACCTAGCAGTCACTTAACAGATGTCGATTGCACATTAGT | |
| | TTTGTCAAGACACCTTTATAACAAAGGGAAATGCACAC | |
| | ACTTCATCCAAAAGGACATTGTGGTTACAGTTAGGAAG | |
| | AAAACACTTAAGATGGAATTATCCATGTAAGTGTAAGT | |
| | TGTTTATATTACAGACAACAAAAGAAAATAGCTCCCAA | |
| | GCTTTGAGCACTGAAGATCTCAGAGGAAGGACTCCTTA | |
| | GGTGCCAATCGTCTAGCAAGCATGCATACATAGGGGAC | |
| | AAGTTAAGAGTCTTACCGGGTTGAGTAAGAAAAGGAAA | |
| | TTATTGCCATGCGAGACAGAGCACGATGCAGCAATGTG | |
| | GAGGATGAGCAGACAGCTAGCATGTTGTAAGGAGCTCA | |
| | GAACGGGAGAGCCTTCCTGCAGTGGCATCGGGGGAGAA | |
| | TGCAAAGGAAAGTCTTGAACAGTCCAGTGAGCCTTTGC | |
| | CTCTGGATAAACTGTTGATACAGGGAGACATTTGCTTTA | |
| | AACAGTCTTCACATAACCAAGCCATTCAGTACTTCTTGA | |
| | AACTGACTTCATAACAGGAGTCATTGTAAGTTCCACAG | |
| | AAAGCAAGACGTATGTATTTCAGTTCTTGTCTTGACCAG | |
| | CAGCACTCCGGAGGCCCAGTGTCCGGTGCCCTCCTTGTA | |
| | TCTGAAGCAGGGGTAACAGCTCTGCTGTGGGCCTGTTTC | |
| | CCTCTAGTATTTACCTCAAGGCTTGGAAATGTATTTTGA | |
| | AAGACCTTCAGTCAAACGAAGTAAAGCAAATGTCAAGA | |
| | AGGATAAACCACTGTTGTGTGTTGATGTGTGGTGTATTG | |
| | TGATGTAAATAAAAAATATCGTTGAAGTTTTACTTTGTA | |
| | AAGATTCTGGTAACACTTGGGTCTCCTTGGGAAAGAAA | |
| | CAACACTTGACCAAAGCATTTAAGTTTTAGGCAAATTCC | |
| | ATAATATTTCACTCTTAGTTATAAAATTATCATACATGA | |
| | GATGTTGAGGATCTATATAGATTATTCAGATTTTATATT | |
| | CCTTATAGATTGACTCAAATATTCCTTAAAAGATTATAT | |
| | ATGATGCTCCTATGGCATAAATTGTAATGTTTAATGTTT | |
| | TGAAAATTGGTTATAGGTATTTTTTAAGTTCCCATAAAG | |
| | TGTTTCTTGTATTTGTCATTGTGAATTATATGTAATTCTA | |
| | ATAGATATTGTTTGCTTTAGTCATTTTTGTCTCCTTTTCC | |
| | TATAGTTTCCTGTGTGAGTGATTCCATGTAATTAATTGG | |
| | ACAGTCTATCCACAGGCAACAAATAGAAGTAGTATTTG | |
| | TAATTGCAGCCTGTGCCAGGGACTGTGTTAGACTGATTA | |
| | GTAACACTATGAATCACCCGAGATCTTAAGTATTGCTAT | |
| | AAAGAGAAGACTGAAGATGAGAGAGTAAAGTATTTTCC | |
| | TGGGATTATATAGTTGGTAAAAAAGAATCATGATTTAA | |
| | ACTCTGGACTTTGGATAAAAGTATCAGTAATTTTCCTCA | |
| | TTGCTAGCAGTCCTCATGCAAGGTCTCTGGACTAAAGC | |
| | GATGTATTACCTGCAAATGCTTGAGTTTCATCCCATACT | |
| | TATGGCGTGAGGTGAGTGTGGCAGCCAGCATCTCTGTC | |
| | CTCCTAGGTAACTGGTTAGTGCTGAACCTCGAGATTCAC | |
| | CACTGTCAGGAGCTGTCTCTGCTTAGAATCACCTGGACA | |
| | GCTGGCTGTCAACCCACGTTAAGTAAGAACATGTGGGA | |
| | CTAGAATTCAGTCAACAATAAAAGCTTTCATCAGGTGG | |
| | CTAAAATGTGCAGACTCTGTTTGTACTGGCATGTTGGCC | |
| | ATCACTCACAGATGCAACTCTAGCCAGAATCAAGAATG | |
| | GGGCAGATCCAGAGCCCAGCTCGCTGAGCTTCTGCCCC | |
| | AGATTTCAGAGGCAGAGTAAGAAGTGGAACAGTGTTTT | |
| | CCTCCCAGGTGCTCAGGACAGGCTGGTCAACTTCCAGT | |
| | CACATATGCCATTTAGTAAGTGCTTCGGCCTGTGCAGCC | |
| | ATTAGTAACCAAATCATGGGATAGTGAATCTAAAATAC | |
| | TCTTGGATAAAATTTGTATTAGAATTAAAAAAAAATGA | |
| | GGGTCAGAAAGGTGAACTAACTCCAACTTAGATGCCCC | |
| | TCCCCTTTATTTTGGTCTCAAATCTGTTTTAATGTGTCCA | |
| | CAACATTAATCAAAAACTGTATTTTTTTTTTTTCGGAGC | |
| | TGGGGACTGAACCCAGGGCCTTGCGCTTGCTAGGCAAG | |
| | CGCTCTACCACTGAGCTAAATCCCCAACCCCCAAAACT | |
| | GTATTTTAAAAATCAGTTCACTTTCATCTTTTTTTCCTTG | |
| | GGTTTTCAGTAAAGACCAGAAGGACTGACAGACAGCTA | |
| | GAGAGGTTGTGTAGACATCCAGATGGGGAGAGTGCATT | |
| | GGACAATCTGAGAAATCTATTCAGGTGCTGCCTAATTCC | |
| | ATTAGCTTTAAGTTAGATACAAGCTGTTTGTAGTATTTT | |
| | GGGTTTTATTGTGCAAATAAACAATAGAGGCCTTTAGTC | |
| | CCAGCGTTAGGATGCAGAGATCCATTTCT | |
| VB220328- | AATGTAGTCTTATGCAATACTCTTGTAGTCTTGCAACAT | 2 |
| 1304ynx | GGTAACGATGAGTTAGCAACATGCCTTACAAGGAGAGA | |
| | AAAAGCACCGTGCATGCCGATTGGTGGAAGTAAGGTGG | |
| | TACGATCGTGCCTTATTAGGAAGGCAACAGACGGGTCT | |
| | GACATGGATTGGACGAACCACTGAATTGCCGCATTGCA | |
| | GAGATATTGTATTTAAGTGCCTAGCTCGATACATAAAC | |
| | GGGTCTCTCTGGTTAGACCAGATCTGAGCCTGGGAGCT | |
| | CTCTGGCTAACTAGGGAACCCACTGCTTAAGCCTCAAT | |
| | AAAGCTTGCCTTGAGTGCTTCAAGTAGTGTGTGCCCGTC | |
| | TGTTGTGTGACTCTGGTAACTAGAGATCCCTCAGACCCT | |
| | TTTAGTCAGTGTGGAAAATCTCTAGCAGTGGCGCCCGA | |
| | ACAGGGACTTGAAAGCGAAAGGGAAACCAGAGGAGCT | |
| | CTCTCGACGCAGGACTCGGCTTGCTGAAGCGCGCACGG | |
| | CAAGAGGCGAGGGGCGGCGACTGGTGAGTACGCCAAA | |
| | AATTTTGACTAGCGGAGGCTAGAAGGAGAGAGATGGGT | |
| | GCGAGAGCGTCAGTATTAAGCGGGGGAGAATTAGATCG | |
| | CGATGGGAAAAAATTCGGTTAAGGCCAGGGGGAAAGA | |
| | AAAAATATAAATTAAAACATATAGTATGGGCAAGCAGG | |
| | GAGCTAGAACGATTCGCAGTTAATCCTGGCCTGTTAGA | |
| | AACATCAGAAGGCTGTAGACAAATACTGGGACAGCTAC | |
| | AACCATCCCTTCAGACAGGATCAGAAGAACTTAGATCA | |
| | TTATATAATACAGTAGCAACCCTCTATTGTGTGCATCAA | |
| | AGGATAGAGATAAAAGACACCAAGGAAGCTTTAGACA | |
| | AGATAGAGGAAGAGCAAAACAAAAGTAAGACCACCGC | |
| | ACAGCAAGCGGCCGCTGATCTTCAGACCTGGAGGAGGA | |
| | GATATGAGGGACAATTGGAGAAGTGAATTATATAAATA | |
| | TAAAGTAGTAAAAATTGAACCATTAGGAGTAGCACCCA | |
| | CCAAGGCAAAGAGAAGAGTGGTGCAGAGAGAAAAAAG | |
| | AGCAGTGGGAATAGGAGCTTTGTTCCTTGGGTTCTTGGG | |
| | AGCAGCAGGAAGCACTATGGGCGCAGCGTCAATGACGC | |
| | TGACGGTACAGGCCAGACAATTATTGTCTGGTATAGTG | |
| | CAGCAGCAGAACAATTTGCTGAGGGCTATTGAGGCGCA | |
| | ACAGCATCTGTTGCAACTCACAGTCTGGGGCATCAAGC | |
| | AGCTCCAGGCAAGAATCCTGGCTGTGGAAAGATACCTA | |
| | AAGGATCAACAGCTCCTGGGGATTTGGGGTTGCTCTGG | |
| | AAAACTCATTTGCACCACTGCTGTGCCTTGGAATGCTAG | |
| | TTGGAGTAATAAATCTCTGGAACAGATTTGGAATCACA | |
| | CGACCTGGATGGAGTGGGACAGAGAAATTAACAATTAC | |
| | ACAAGCTTAATACACTCCTTAATTGAAGAATCGCAAAA | |
| | CCAGCAAGAAAAGAATGAACAAGAATTATTGGAATTAG | |
| | ATAAATGGGCAAGTTTGTGGAATTGGTTTAACATAACA | |
| | AATTGGCTGTGGTATATAAAATTATTCATAATGATAGTA | |
| | GGAGGCTTGGTAGGTTTAAGAATAGTTTTTGCTGTACTT | |
| | TCTATAGTGAATAGAGTTAGGCAGGGATATTCACCATT | |
| | ATCGTTTCAGACCCACCTCCCAACCCCGAGGGGACCCG | |
| | ACAGGCCCGAAGGAATAGAAGAAGAAGGTGGAGAGAG | |
| | AGACAGAGACAGATCCATTCGATTAGTGAACGGATCTC | |
| | GACGGTATCGCTAGCTTTTAAAAGAAAAGGGGGGATTG | |
| | GGGGGTACAGTGCAGGGGAAAGAATAGTAGACATAAT | |
| | AGCAACAGACATACAAACTAAAGAATTACAAAAACAA | |
| | ATTACAAAAATTCAAAATTTTACTAGTATCAACTTTGTA | |
| | TAGAAAAGTTGCTGCAGAGGGCCCTGCGTATGAGTGCA | |
| | AGTGGGTTTTAGGACCAGGATGAGGCGGGGGGGGGTG | |
| | CCTACCTGACGACCGACCCCGACCCACTGGACAAGCAC | |
| | CCAACCCCCATTCCCCAAATTGCGCATCCCCTATCAGAG | |
| | AGGGGGAGGGGAAACAGGATGCGGCGAGGCGCGTGCG | |
| | CACTGCCAGCTTCAGCACCGCGGACAGTGCCTTCGCCC | |
| | CCGCCTGGCGGCGCGCGCCACCGCCGCCTCAGCACTGA | |
| | AGGCGCGCTGACGTCACTCGCCGGTCCCCCGCAAACTC | |
| | CCCTTCCCGGCCACCTTGGTCGCGTCCGCGCCGCCGCCG | |
| | GCCCAGCCGGACCGCACCACGCGAGGCGCGAGATAGG | |
| | GGGGCACGGGCGCGACCATCTGCGCTGCGGCGCCGGCG | |
| | ACTCAGCGCTGCCTCAGTCTGCGGTGGGCAGCGGAGGA | |
| | GTCGTGTCGTGCCTGAGAGCGCAGCAAGTTTGTACAAA | |
| | AAAGCAGGCTGCCACCATGTTCCTGAGGGGGTATAAGA | |
| | GAGAGGGGCAGGTCAGGAAGCATAGGAAGTCAGTTGC | |
| | ACATGGGGGAGGGTTGTGGGGAAACTGTAAAAGCTATG | |
| | CTGGGGAGGCGGCACGGAGCTTGATTCACCTTCACCTG | |
| | CGCCGGGCACCCGCTGACCCCGGGTTTCCGCCCGGAGA | |
| | GCAGTCAGATATGAACGGACGGGTGGATTATTTAGTCT | |
| | CCGAGGAAGAGATCAACCTGACCAGAGGACCCTCGGG | |
| | GCTGGGCTTCAACATCGTCGGTGGGACAGATCAACAGT | |
| | ATGTCTCCAATGACAGTGGCATCTACGTCAGCCGCATC | |
| | AAAGAGGATGGGGCTGCGGCCCGGGATGGGCGGCTCC | |
| | AGGAGGGTGATAAGATCCTCTCGGTAAATGGCCAAGAC | |
| | CTGAAGAACCTGTTGCACCAAGATGCCGTAGACCTCTT | |
| | CCGTAATGCAGGATATGCCGTGTCCCTGAGAGTGCAGC | |
| | ACAGGTTACCAGTGCAGAATGGACCTATAGTTCATCGA | |
| | GGCGACGGAGAGCCGAGTGGAGTTCCTGTAGCTGTGGT | |
| | GCTGCTGCCAGTGTTTGCCCTTACCCTGGTAGCAGTTTG | |
| | GGCCTTCGTGAGATACCGAAAGCAGCTCTGAACCCAGC | |
| | TTTCTTGTACAAAGTGGGCCCCTCTCCCTCCCCCCCCCC | |
| | TAACGTTACTGGCCGAAGCCGCTTGGAATAAGGCCGGT | |
| | GTGCGTTTGTCTATATGTTATTTTCCACCATATTGCCGTC | |
| | TTTTGGCAATGTGAGGGCCCGGAAACCTGGCCCTGTCTT | |
| | CTTGACGAGCATTCCTAGGGGTCTTTCCCCTCTCGCCAA | |
| | AGGAATGCAAGGTCTGTTGAATGTCGTGAAGGAAGCAG | |
| | TTCCTCTGGAAGCTTCTTGAAGACAAACAACGTCTGTAG | |
| | CGACCCTTTGCAGGCAGCGGAACCCCCCACCTGGCGAC | |
| | AGGTGCCTCTGCGGCCAAAAGCCACGTGTATAAGATAC | |
| | ACCTGCAAAGGCGGCACAACCCCAGTGCCACGTTGTGA | |
| | GTTGGATAGTTGTGGAAAGAGTCAAATGGCTCTCCTCA | |
| | AGCGTATTCAACAAGGGGCTGAAGGATGCCCAGAAGGT | |
| | ACCCCATTGTATGGGATCTGATCTGGGGCCTCGGTGCAC | |
| | ATGCTTTACATGTGTTTAGTCGAGGTTAAAAAAACGTCT | |
| | AGGCCCCCCGAACCACGGGGACGTGGTTTTCCTTTGAA | |
| | AAACACGATGATAATATGGCCACAACCATGGTGAGCAA | |
| | GGGCGAGGAGCTGTTCACCGGGGTGGTGCCCATCCTGG | |
| | TCGAGCTGGACGGCGACGTAAACGGCCACAAGTTCAGC | |
| | GTGTCCGGCGAGGGCGAGGGCGATGCCACCTACGGCAA | |
| | GCTGACCCTGAAGTTCATCTGCACCACCGGCAAGCTGC | |
| | CCGTGCCCTGGCCCACCCTCGTGACCACCTTCACCTACG | |
| | GCGTGCAGTGCTTCGCCCGCTACCCCGACCACATGAAG | |
| | CAGCACGACTTCTTCAAGTCCGCCATGCCCGAAGGCTA | |
| | CGTCCAGGAGCGCACCATCTTCTTCAAGGACGACGGCA | |
| | ACTACAAGACCCGCGCCGAGGTGAAGTTCGAGGGCGAC | |
| | ACCCTGGTGAACCGCATCGAGCTGAAGGGCATCGACTT | |
| | CAAGGAGGACGGCAACATCCTGGGGCACAAGCTGGAG | |
| | TACAACTACAACAGCCACAAGGTCTATATCACCGCCGA | |
| | CAAGCAGAAGAACGGCATCAAGGTGAACTTCAAGACCC | |
| | GCCACAACATCGAGGACGGCAGCGTGCAGCTCGCCGAC | |
| | CACTACCAGCAGAACACCCCCATCGGCGACGGCCCCGT | |
| | GCTGCTGCCCGACAACCACTACCTGAGCACCCAGTCCG | |
| | CCCTGAGCAAAGACCCCAACGAGAAGCGCGATCACATG | |
| | GTCCTGCTGGAGTTCGTGACCGCCGCCGGGATCACTCTC | |
| | GGCATGGACGAGCTGTACAAGTAACAACTTTATTATAC | |
| | ATAGTTGATCAATTCCGATAATCAACCTCTGGATTACAA | |
| | AATTTGTGAAAGATTGACTGGTATTCTTAACTATGTTGC | |
| | TCCTTTTACGCTATGTGGATACGCTGCTTTAATGCCTTT | |
| | GTATCATGCTATTGCTTCCCGTATGGCTTTCATTTTCTCC | |
| | TCCTTGTATAAATCCTGGTTGCTGTCTCTTTATGAGGAG | |
| | TTGTGGCCCGTTGTCAGGCAACGTGGCGTGGTGTGCACT | |
| | GTGTTTGCTGACGCAACCCCCACTGGTTGGGGCATTGCC | |
| | ACCACCTGTCAGCTCCTTTCCGGGACTTTCGCTTTCCCC | |
| | CTCCCTATTGCCACGGCGGAACTCATCGCCGCCTGCCTT | |
| | GCCCGCTGCTGGACAGGGGCTCGGCTGTTGGGCACTGA | |
| | CAATTCCGTGGTGTTGTCGGGGAAGCTGACGTCCTTTCC | |
| | ATGGCTGCTCGCCTGTGTTGCCACCTGGATTCTGCGCGG | |
| | GACGTCCTTCTGCTACGTCCCTTCGGCCCTCAATCCAGC | |
| | GGACCTTCCTTCCCGCGGCCTGCTGCCGGCTCTGCGGCC | |
| | TCTTCCGCGTCTTCGCCTTCGCCCTCAGACGAGTCGGAT | |
| | CTCCCTTTGGGCCGCCTCCCCGCATCGGGAATTCCCGCG | |
| | GTTCGCTTTAAGACCAATGACTTACAAGGCAGCTGTAG | |
| | ATCTTAGCCACTTTTTAAAAGAAAAGGGGGGACTGGAA | |
| | GGGCTAATTCACTCCCAACGAAGACAAGATCTGCTTTTT | |
| | GCTTGTACTGGGTCTCTCTGGTTAGACCAGATCTGAGCC | |
| | TGGGAGCTCTCTGGCTAACTAGGGAACCCACTGCTTAA | |
| | GCCTCAATAAAGCTTGCCTTGAGTGCTTCAAGTAGTGTG | |
| | TGCCCGTCTGTTGTGTGACTCTGGTAACTAGAGATCCCT | |
| | CAGACCCTTTTAGTCAGTGTGGAAAATCTCTAGCAGTA | |
| | GTAGTTCATGTCATCTTATTATTCAGTATTTATAACTTGC | |
| | AAAGAAATGAATATCAGAGAGTGAGAGGAACTTGTTTA | |
| | TTGCAGCTTATAATGGTTACAAATAAAGCAATAGCATC | |
| | ACAAATTTCACAAATAAAGCATTTTTTTCACTGCATTCT | |
| | AGTTGTGGTTTGTCCAAACTCATCAATGTATCTTATCAT | |
| | GTCTGGCTCTAGCTATCCCGCCCCTAACTCCGCCCATCC | |
| | CGCCCCTAACTCCGCCCAGTTCCGCCCATTCTCCGCCCC | |
| | ATGGCTGACTAATTTTTTTTATTTATGCAGAGGCCGAGG | |
| | CCGCCTCGGCCTCTGAGCTATTCCAGAAGTAGTGAGGA | |
| | GGCTTTTTTGGAGGCCTAGGGACGTACCCAATTCGCCCT | |
| | ATAGTGAGTCGTATTACGCGCGCTCACTGGCCGTCGTTT | |
| | TACAACGTCGTGACTGGGAAAACCCTGGCGTTACCCAA | |
| | CTTAATCGCCTTGCAGCACATCCCCCTTTCGCCAGCTGG | |
| | CGTAATAGCGAAGAGGCCCGCACCGATCGCCCTTCCCA | |
| | ACAGTTGCGCAGCCTGAATGGCGAATGGGACGCGCCCT | |
| | GTAGCGGCGCATTAAGCGCGGCGGGTGTGGTGGTTACG | |
| | CGCAGCGTGACCGCTACACTTGCCAGCGCCCTAGCGCC | |
| | CGCTCCTTTCGCTTTCTTCCCTTCCTTTCTCGCCACGTTC | |
| | GCCGGCTTTCCCCGTCAAGCTCTAAATCGGGGGCTCCCT | |
| | TTAGGGTTCCGATTTAGTGCTTTACGGCACCTCGACCCC | |
| | AAAAAACTTGATTAGGGTGATGGTTCACGTAGTGGGCC | |
| | ATCGCCCTGATAGACGGTTTTTCGCCCTTTGACGTTGGA | |
| | GTCCACGTTCTTTAATAGTGGACTCTTGTTCCAAACTGG | |
| | AACAACACTCAACCCTATCTCGGTCTATTCTTTTGATTT | |
| | ATAAGGGATTTTGCCGATTTCGGCCTATTGGTTAAAAAA | |
| | TGAGCTGATTTAACAAAAATTTAACGCGAATTTTAACA | |
| | AAATATTAACGCTTACAATTTAGGTGGCACTTTTCGGGG | |
| | AAATGTGCGCGGAACCCCTATTTGTTTATTTTTCTAAAT | |
| | ACATTCAAATATGTATCCGCTCATGAGACAATAACCCT | |
| | GATAAATGCTTCAATAATATTGAAAAAGGAAGAGTATG | |
| | AGTATTCAACATTTCCGTGTCGCCCTTATTCCCTTTTTTG | |
| | CGGCATTTTGCCTTCCTGTTTTTGCTCACCCAGAAACGC | |
| | TGGTGAAAGTAAAAGATGCTGAAGATCAGTTGGGTGCA | |
| | CGAGTGGGTTACATCGAACTGGATCTCAACAGCGGTAA | |
| | GATCCTTGAGAGTTTTCGCCCCGAAGAACGTTTTCCAAT | |
| | GATGAGCACTTTTAAAGTTCTGCTATGTGGCGCGGTATT | |
| | ATCCCGTATTGACGCCGGGCAAGAGCAACTCGGTCGCC | |
| | GCATACACTATTCTCAGAATGACTTGGTTGAGTACTCAC | |
| | CAGTCACAGAAAAGCATCTTACGGATGGCATGACAGTA | |
| | AGAGAATTATGCAGTGCTGCCATAACCATGAGTGATAA | |
| | CACTGCGGCCAACTTACTTCTGACAACGATCGGAGGAC | |
| | CGAAGGAGCTAACCGCTTTTTTGCACAACATGGGGGAT | |
| | CATGTAACTCGCCTTGATCGTTGGGAACCGGAGCTGAA | |
| | TGAAGCCATACCAAACGACGAGCGTGACACCACGATGC | |
| | CTGTAGCAATGGCAACAACGTTGCGCAAACTATTAACT | |
| | GGCGAACTACTTACTCTAGCTTCCCGGCAACAATTAATA | |
| | GACTGGATGGAGGCGGATAAAGTTGCAGGACCACTTCT | |
| | GCGCTCGGCCCTTCCGGCTGGCTGGTTTATTGCTGATAA | |
| | ATCTGGAGCCGGTGAGCGTGGGTCTCGCGGTATCATTG | |
| | CAGCACTGGGGCCAGATGGTAAGCCCTCCCGTATCGTA | |
| | GTTATCTACACGACGGGGAGTCAGGCAACTATGGATGA | |
| | ACGAAATAGACAGATCGCTGAGATAGGTGCCTCACTGA | |
| | TTAAGCATTGGTAACTGTCAGACCAAGTTTACTCATATA | |
| | TACTTTAGATTGATTTAAAACTTCATTTTTAATTTAAAA | |
| | GGATCTAGGTGAAGATCCTTTTTGATAATCTCATGACCA | |
| | AAATCCCTTAACGTGAGTTTTCGTTCCACTGAGCGTCAG | |
| | ACCCCGTAGAAAAGATCAAAGGATCTTCTTGAGATCCT | |
| | TTTTTTCTGCGCGTAATCTGCTGCTTGCAAACAAAAAAA | |
| | CCACCGCTACCAGCGGTGGTTTGTTTGCCGGATCAAGA | |
| | GCTACCAACTCTTTTTCCGAAGGTAACTGGCTTCAGCAG | |
| | AGCGCAGATACCAAATACTGTTCTTCTAGTGTAGCCGTA | |
| | GTTAGGCCACCACTTCAAGAACTCTGTAGCACCGCCTA | |
| | CATACCTCGCTCTGCTAATCCTGTTACCAGTGGCTGCTG | |
| | CCAGTGGCGATAAGTCGTGTCTTACCGGGTTGGACTCA | |
| | AGACGATAGTTACCGGATAAGGCGCAGCGGTCGGGCTG | |
| | AACGGGGGGTTCGTGCACACAGCCCAGCTTGGAGCGAA | |
| | CGACCTACACCGAACTGAGATACCTACAGCGTGAGCTA | |
| | TGAGAAAGCGCCACGCTTCCCGAAGAGAGAAAGGCGG | |
| | ACAGGTATCCGGTAAGCGGCAGGGTCGGAACAGGAGA | |
| | GCGCACGAGGGAGCTTCCAGGGGGAAACGCCTGGTATC | |
| | TTTATAGTCCTGTCGGGTTTCGCCACCTCTGACTTGAGC | |
| | GTCGATTTTTGTGATGCTCGTCAGGGGGGCGGAGCCTAT | |
| | GGAAAAACGCCAGCAACGCGGCCTTTTTACGGTTCCTG | |
| | GCCTTTTGCTGGCCTTTTGCTCACATGTTCTTTCCTGCGT | |
| | TATCCCCTGATTCTGTGGATAACCGTATTACCGCCTTTG | |
| | AGTGAGCTGATACCGCTCGCCGCAGCCGAACGACCGAG | |
| | CGCAGCGAGTCAGTGAGCGAGGAAGCGGAAGAGCGCC | |
| | CAATACGCAAACCGCCTCTCCCCGCGCGTTGGCCGATTC | |
| | ATTAATGCAGCTGGCACGACAGGTTTCCCGACTGGAAA | |
| | GCGGGCAGTGAGCGCAACGCAATTAATGTGAGTTAGCT | |
| | CACTCATTAGGCACCCCAGGCTTTACACTTTATGCTTCC | |
| | GGCTCGTATGTTGTGTGGAATTGTGAGCGGATAACAAT | |
| | TTCACACAGGAAACAGCTATGACCATGATTACGCCAAG | |
| | CGCGCAATTAACCCTCACTAAAGGGAACAAAAGCTGGA | |
| | GCTGCAAGCTT | |
| Map of the | ATGGAGTTCCCGTTCGATGTGGACGCGCTGTTCCCGGA | 3 |
| newly generated | GCGGATCACGGTGCTGGACCAGCACCTGAGGCCCCCAG | |
| lentiviral | CCCGCCGACCCGGAACCACAACGCCGGCCCGTGTTGAT | |
| plasmid | CTACAGCAGCAAATTATGACCATTATAGATGAACTGGG | |
| expressing | CAAGGCTTCTGCCAAGGCCCAGAATCTTTCCGCTCCTAT | |
| ATAT1 fused | CACTAGTGCATCAAGGATGCAGAGTAACCGCCATGTTG | |
| to the | TTTATATTCTCAAAGACAGTTCAGCCCGACCGGCTGGA | |
| mitochondrial | AAAGGAGCCATTATTGGTTTCATCAAAGTTGGATACAA | |
| targeting | GAAGCTCTTTGTACTGGATGATCGTGAGGCTCATAATG | |
| OMP25 domain | AGGTAGAACCACTTTGCATCCTGGACTTTTACATCCATG | |
| NM_024909.5 | AGTCTGTGCAACGCCATGGCCATGGGCGAGAACTCTTC | |
| Homo sapiens | CAGTATATGTTGCAGAAGGAGCGAGTGGAACCGCACCA | |
| alpha tubulin | ACTGGCAATTGACCGACCCTCACAGAAGCTGCTGAAAT | |
| acetyl | TCCTGAATAAGCACTACAATCTGGAGACCACAGTCCCA | |
| transferase 1 | CAGGTGAACAACTTTGTGATCTTTGAAGGCTTCTTTGCC | |
| (ATAT1), | CATCAACATCGGCCCCCTGCTCCCTCTCTGAGGGCAACT | |
| transcript | CGACACTCTCGTGCTGCTGCAGTCGATCCCACGCCCGCT | |
| variant 2, | GCTCCAGCAAGGAAGCTGCCACCCAAGAGAGCAGAGG | |
| mRNA | GAGACATCAAGCCATACTCCTCTAGTGACCGAGAATTT | |
| (underlined) | CTGAAGGTAGCTGTGGAGCCTCCTTGGCCCCTAAACAG | |
| NM_022599.2 | GGCCCCTCGCCGCGCCACACCTCCAGCCCACCCACCCC | |
| Rattus | CCCGCTCCAGCAGCCTGGGAAACTCACCAGAACGAGGT | |
| norvegicus | CCCCTCCGCCCCTTTGTGCCAGAGCAGGAGCTGCTGCGT | |
| synaptojanin 2 | TCCTTGCGCCTCTGCCCCCCACACCCTACCGCCCGCCTT | |
| binding protein | CTGTTGGCTGCTGACCCTGGGGGCAGCCCAGCTCAACG | |
| (Synj2bp), | TCGTCGCACCAGCTCCCTTCCCCGCTCTGAGGAGAGTCG | |
| mRNA (bold) | ATACCATCGAGGCGACGGAGAGCCGAGTGGAGTTCC | |
| | TGTAGCTGTGGTGCTGCTGCCAGTGTTTGCCCTTAC | |
| | CCTGGTAGCAGTTTGGGCCTTCGTGAGATACCGAAA | |
| | GCAGCTCTGA | |
| VB220329- | AATGTAGTCTTATGCAATACTCTTGTAGTCTTGCAACAT | 4 |
| 1381pbx | GGTAACGATGAGTTAGCAACATGCCTTACAAGGAGAGA | |
| | AAAAGCACCGTGCATGCCGATTGGTGGAAGTAAGGTGG | |
| | TACGATCGTGCCTTATTAGGAAGGCAACAGACGGGTCT | |
| | GACATGGATTGGACGAACCACTGAATTGCCGCATTGCA | |
| | GAGATATTGTATTTAAGTGCCTAGCTCGATACATAAAC | |
| | GGGTCTCTCTGGTTAGACCAGATCTGAGCCTGGGAGCT | |
| | CTCTGGCTAACTAGGGAACCCACTGCTTAAGCCTCAAT | |
| | AAAGCTTGCCTTGAGTGCTTCAAGTAGTGTGTGCCCGTC | |
| | TGTTGTGTGACTCTGGTAACTAGAGATCCCTCAGACCCT | |
| | TTTAGTCAGTGTGGAAAATCTCTAGCAGTGGCGCCCGA | |
| | ACAGGGACTTGAAAGCGAAAGGGAAACCAGAGGAGCT | |
| | CTCTCGACGCAGGACTCGGCTTGCTGAAGCGCGCACGG | |
| | CAAGAGGCGAGGGGCGGCGACTGGTGAGTACGCCAAA | |
| | AATTTTGACTAGCGGAGGCTAGAAGGAGAGAGATGGGT | |
| | GCGAGAGCGTCAGTATTAAGCGGGGGAGAATTAGATCG | |
| | CGATGGGAAAAAATTCGGTTAAGGCCAGGGGGAAAGA | |
| | AAAAATATAAATTAAAACATATAGTATGGGCAAGCAGG | |
| | GAGCTAGAACGATTCGCAGTTAATCCTGGCCTGTTAGA | |
| | AACATCAGAAGGCTGTAGACAAATACTGGGACAGCTAC | |
| | AACCATCCCTTCAGACAGGATCAGAAGAACTTAGATCA | |
| | TTATATAATACAGTAGCAACCCTCTATTGTGTGCATCAA | |
| | AGGATAGAGATAAAAGACACCAAGGAAGCTTTAGACA | |
| | AGATAGAGGAAGAGCAAAACAAAAGTAAGACCACCGC | |
| | ACAGCAAGCGGCCGCTGATCTTCAGACCTGGAGGAGGA | |
| | GATATGAGGGACAATTGGAGAAGTGAATTATATAAATA | |
| | TAAAGTAGTAAAAATTGAACCATTAGGAGTAGCACCCA | |
| | CCAAGGCAAAGAGAAGAGTGGTGCAGAGAGAAAAAAG | |
| | AGCAGTGGGAATAGGAGCTTTGTTCCTTGGGTTCTTGGG | |
| | AGCAGCAGGAAGCACTATGGGCGCAGCGTCAATGACGC | |
| | TGACGGTACAGGCCAGACAATTATTGTCTGGTATAGTG | |
| | CAGCAGCAGAACAATTTGCTGAGGGCTATTGAGGCGCA | |
| | ACAGCATCTGTTGCAACTCACAGTCTGGGGCATCAAGC | |
| | AGCTCCAGGCAAGAATCCTGGCTGTGGAAAGATACCTA | |
| | AAGGATCAACAGCTCCTGGGGATTTGGGGTTGCTCTGG | |
| | AAAACTCATTTGCACCACTGCTGTGCCTTGGAATGCTAG | |
| | TTGGAGTAATAAATCTCTGGAACAGATTTGGAATCACA | |
| | CGACCTGGATGGAGTGGGACAGAGAAATTAACAATTAC | |
| | ACAAGCTTAATACACTCCTTAATTGAAGAATCGCAAAA | |
| | CCAGCAAGAAAAGAATGAACAAGAATTATTGGAATTAG | |
| | ATAAATGGGCAAGTTTGTGGAATTGGTTTAACATAACA | |
| | AATTGGCTGTGGTATATAAAATTATTCATAATGATAGTA | |
| | GGAGGCTTGGTAGGTTTAAGAATAGTTTTTGCTGTACTT | |
| | TCTATAGTGAATAGAGTTAGGCAGGGATATTCACCATT | |
| | ATCGTTTCAGACCCACCTCCCAACCCCGAGGGGACCCG | |
| | ACAGGCCCGAAGGAATAGAAGAAGAAGGTGGAGAGAG | |
| | AGACAGAGACAGATCCATTCGATTAGTGAACGGATCTC | |
| | GACGGTATCGCTAGCTTTTAAAAGAAAAGGGGGGATTG | |
| | GGGGGTACAGTGCAGGGGAAAGAATAGTAGACATAAT | |
| | AGCAACAGACATACAAACTAAAGAATTACAAAAACAA | |
| | ATTACAAAAATTCAAAATTTTACTAGTATCAACTTTGTA | |
| | TAGAAAAGTTGCTGCAGAGGGCCCTGCGTATGAGTGCA | |
| | AGTGGGTTTTAGGACCAGGATGAGGCGGGGGGGGGTG | |
| | CCTACCTGACGACCGACCCCGACCCACTGGACAAGCAC | |
| | CCAACCCCCATTCCCCAAATTGCGCATCCCCTATCAGAG | |
| | AGGGGGAGGGGAAACAGGATGCGGCGAGGCGCGTGCG | |
| | CACTGCCAGCTTCAGCACCGCGGACAGTGCCTTCGCCC | |
| | CCGCCTGGCGGCGCGCGCCACCGCCGCCTCAGCACTGA | |
| | AGGCGCGCTGACGTCACTCGCCGGTCCCCCGCAAACTC | |
| | CCCTTCCCGGCCACCTTGGTCGCGTCCGCGCCGCCGCCG | |
| | GCCCAGCCGGACCGCACCACGCGAGGCGCGAGATAGG | |
| | GGGGCACGGGCGCGACCATCTGCGCTGCGGCGCCGGCG | |
| | ACTCAGCGCTGCCTCAGTCTGCGGTGGGCAGCGGAGGA | |
| | GTCGTGTCGTGCCTGAGAGCGCAGCAAGTTTGTACAAA | |
| | AAAGCAGGCTGCCACCATGGAGTTCCCGTTCGATGTGG | |
| | ACGCGCTGTTCCCGGAGCGGATCACGGTGCTGGACCAG | |
| | CACCTGAGGCCCCCAGCCCGCCGACCCGGAACCACAAC | |
| | GCCGGCCCGTGTTGATCTACAGCAGCAAATTATGACCA | |
| | TTATAGATGAACTGGGCAAGGCTTCTGCCAAGGCCCAG | |
| | AATCTTTCCGCTCCTATCACTAGTGCATCAAGGATGCAG | |
| | AGTAACCGCCATGTTGTTTATATTCTCAAAGACAGTTCA | |
| | GCCCGACCGGCTGGAAAAGGAGCCATTATTGGTTTCAT | |
| | CAAAGTTGGATACAAGAAGCTCTTTGTACTGGATGATC | |
| | GTGAGGCTCATAATGAGGTAGAACCACTTTGCATCCTG | |
| | GACTTTTACATCCATGAGTCTGTGCAACGCCATGGCCAT | |
| | GGGCGAGAACTCTTCCAGTATATGTTGCAGAAGGAGCG | |
| | AGTGGAACCGCACCAACTGGCAATTGACCGACCCTCAC | |
| | AGAAGCTGCTGAAATTCCTGAATAAGCACTACAATCTG | |
| | GAGACCACAGTCCCACAGGTGAACAACTTTGTGATCTT | |
| | TGAAGGCTTCTTTGCCCATCAACATCGGCCCCCTGCTCC | |
| | CTCTCTGAGGGCAACTCGACACTCTCGTGCTGCTGCAGT | |
| | CGATCCCACGCCCGCTGCTCCAGCAAGGAAGCTGCCAC | |
| | CCAAGAGAGCAGAGGGAGACATCAAGCCATACTCCTCT | |
| | AGTGACCGAGAATTTCTGAAGGTAGCTGTGGAGCCTCC | |
| | TTGGCCCCTAAACAGGGCCCCTCGCCGCGCCACACCTC | |
| | CAGCCCACCCACCCCCCCGCTCCAGCAGCCTGGGAAAC | |
| | TCACCAGAACGAGGTCCCCTCCGCCCCTTTGTGCCAGA | |
| | GCAGGAGCTGCTGCGTTCCTTGCGCCTCTGCCCCCCACA | |
| | CCCTACCGCCCGCCTTCTGTTGGCTGCTGACCCTGGGGG | |
| | CAGCCCAGCTCAACGTCGTCGCACCAGCTCCCTTCCCCG | |
| | CTCTGAGGAGAGTCGATACCATCGAGGCGACGGAGAGC | |
| | CGAGTGGAGTTCCTGTAGCTGTGGTGCTGCTGCCAGTGT | |
| | TTGCCCTTACCCTGGTAGCAGTTTGGGCCTTCGTGAGAT | |
| | ACCGAAAGCAGCTCTGAACCCAGCTTTCTTGTACAAAG | |
| | TGGGCCCCTCTCCCTCCCCCCCCCCTAACGTTACTGGCC | |
| | GAAGCCGCTTGGAATAAGGCCGGTGTGCGTTTGTCTAT | |
| | ATGTTATTTTCCACCATATTGCCGTCTTTTGGCAATGTG | |
| | AGGGCCCGGAAACCTGGCCCTGTCTTCTTGACGAGCAT | |
| | TCCTAGGGGTCTTTCCCCTCTCGCCAAAGGAATGCAAG | |
| | GTCTGTTGAATGTCGTGAAGGAAGCAGTTCCTCTGGAA | |
| | GCTTCTTGAAGACAAACAACGTCTGTAGCGACCCTTTGC | |
| | AGGCAGCGGAACCCCCCACCTGGCGACAGGTGCCTCTG | |
| | CGGCCAAAAGCCACGTGTATAAGATACACCTGCAAAGG | |
| | CGGCACAACCCCAGTGCCACGTTGTGAGTTGGATAGTT | |
| | GTGGAAAGAGTCAAATGGCTCTCCTCAAGCGTATTCAA | |
| | CAAGGGGCTGAAGGATGCCCAGAAGGTACCCCATTGTA | |
| | TGGGATCTGATCTGGGGCCTCGGTGCACATGCTTTACAT | |
| | GTGTTTAGTCGAGGTTAAAAAAACGTCTAGGCCCCCCG | |
| | AACCACGGGGACGTGGTTTTCCTTTGAAAAACACGATG | |
| | ATAATATGGCCACAACCATGGTGAGCAAGGGCGAGGA | |
| | GCTGTTCACCGGGGTGGTGCCCATCCTGGTCGAGCTGG | |
| | ACGGCGACGTAAACGGCCACAAGTTCAGCGTGTCCGGC | |
| | GAGGGCGAGGGCGATGCCACCTACGGCAAGCTGACCCT | |
| | GAAGTTCATCTGCACCACCGGCAAGCTGCCCGTGCCCT | |
| | GGCCCACCCTCGTGACCACCTTCACCTACGGCGTGCAGT | |
| | GCTTCGCCCGCTACCCCGACCACATGAAGCAGCACGAC | |
| | TTCTTCAAGTCCGCCATGCCCGAAGGCTACGTCCAGGA | |
| | GCGCACCATCTTCTTCAAGGACGACGGCAACTACAAGA | |
| | CCCGCGCCGAGGTGAAGTTCGAGGGCGACACCCTGGTG | |
| | AACCGCATCGAGCTGAAGGGCATCGACTTCAAGGAGGA | |
| | CGGCAACATCCTGGGGCACAAGCTGGAGTACAACTACA | |
| | ACAGCCACAAGGTCTATATCACCGCCGACAAGCAGAAG | |
| | AACGGCATCAAGGTGAACTTCAAGACCCGCCACAACAT | |
| | CGAGGACGGCAGCGTGCAGCTCGCCGACCACTACCAGC | |
| | AGAACACCCCCATCGGCGACGGCCCCGTGCTGCTGCCC | |
| | GACAACCACTACCTGAGCACCCAGTCCGCCCTGAGCAA | |
| | AGACCCCAACGAGAAGCGCGATCACATGGTCCTGCTGG | |
| | AGTTCGTGACCGCCGCCGGGATCACTCTCGGCATGGAC | |
| | GAGCTGTACAAGTAACAACTTTATTATACATAGTTGATC | |
| | AATTCCGATAATCAACCTCTGGATTACAAAATTTGTGAA | |
| | AGATTGACTGGTATTCTTAACTATGTTGCTCCTTTTACG | |
| | CTATGTGGATACGCTGCTTTAATGCCTTTGTATCATGCT | |
| | ATTGCTTCCCGTATGGCTTTCATTTTCTCCTCCTTGTATA | |
| | AATCCTGGTTGCTGTCTCTTTATGAGGAGTTGTGGCCCG | |
| | TTGTCAGGCAACGTGGCGTGGTGTGCACTGTGTTTGCTG | |
| | ACGCAACCCCCACTGGTTGGGGCATTGCCACCACCTGT | |
| | CAGCTCCTTTCCGGGACTTTCGCTTTCCCCCTCCCTATTG | |
| | CCACGGCGGAACTCATCGCCGCCTGCCTTGCCCGCTGCT | |
| | GGACAGGGGCTCGGCTGTTGGGCACTGACAATTCCGTG | |
| | GTGTTGTCGGGGAAGCTGACGTCCTTTCCATGGCTGCTC | |
| | GCCTGTGTTGCCACCTGGATTCTGCGCGGGACGTCCTTC | |
| | TGCTACGTCCCTTCGGCCCTCAATCCAGCGGACCTTCCT | |
| | TCCCGCGGCCTGCTGCCGGCTCTGCGGCCTCTTCCGCGT | |
| | CTTCGCCTTCGCCCTCAGACGAGTCGGATCTCCCTTTGG | |
| | GCCGCCTCCCCGCATCGGGAATTCCCGCGGTTCGCTTTA | |
| | AGACCAATGACTTACAAGGCAGCTGTAGATCTTAGCCA | |
| | CTTTTTAAAAGAAAAGGGGGGACTGGAAGGGCTAATTC | |
| | ACTCCCAACGAAGACAAGATCTGCTTTTTGCTTGTACTG | |
| | GGTCTCTCTGGTTAGACCAGATCTGAGCCTGGGAGCTCT | |
| | CTGGCTAACTAGGGAACCCACTGCTTAAGCCTCAATAA | |
| | AGCTTGCCTTGAGTGCTTCAAGTAGTGTGTGCCCGTCTG | |
| | TTGTGTGACTCTGGTAACTAGAGATCCCTCAGACCCTTT | |
| | TAGTCAGTGTGGAAAATCTCTAGCAGTAGTAGTTCATGT | |
| | CATCTTATTATTCAGTATTTATAACTTGCAAAGAAATGA | |
| | ATATCAGAGAGTGAGAGGAACTTGTTTATTGCAGCTTA | |
| | TAATGGTTACAAATAAAGCAATAGCATCACAAATTTCA | |
| | CAAATAAAGCATTTTTTTCACTGCATTCTAGTTGTGGTT | |
| | TGTCCAAACTCATCAATGTATCTTATCATGTCTGGCTCT | |
| | AGCTATCCCGCCCCTAACTCCGCCCATCCCGCCCCTAAC | |
| | TCCGCCCAGTTCCGCCCATTCTCCGCCCCATGGCTGACT | |
| | AATTTTTTTTATTTATGCAGAGGCCGAGGCCGCCTCGGC | |
| | CTCTGAGCTATTCCAGAAGTAGTGAGGAGGCTTTTTTGG | |
| | AGGCCTAGGGACGTACCCAATTCGCCCTATAGTGAGTC | |
| | GTATTACGCGCGCTCACTGGCCGTCGTTTTACAACGTCG | |
| | TGACTGGGAAAACCCTGGCGTTACCCAACTTAATCGCC | |
| | TTGCAGCACATCCCCCTTTCGCCAGCTGGCGTAATAGCG | |
| | AAGAGGCCCGCACCGATCGCCCTTCCCAACAGTTGCGC | |
| | AGCCTGAATGGCGAATGGGACGCGCCCTGTAGCGGCGC | |
| | ATTAAGCGCGGCGGGTGTGGTGGTTACGCGCAGCGTGA | |
| | CCGCTACACTTGCCAGCGCCCTAGCGCCCGCTCCTTTCG | |
| | CTTTCTTCCCTTCCTTTCTCGCCACGTTCGCCGGCTTTCC | |
| | CCGTCAAGCTCTAAATCGGGGGCTCCCTTTAGGGTTCCG | |
| | ATTTAGTGCTTTACGGCACCTCGACCCCAAAAAACTTGA | |
| | TTAGGGTGATGGTTCACGTAGTGGGCCATCGCCCTGAT | |
| | AGACGGTTTTTCGCCCTTTGACGTTGGAGTCCACGTTCT | |
| | TTAATAGTGGACTCTTGTTCCAAACTGGAACAACACTCA | |
| | ACCCTATCTCGGTCTATTCTTTTGATTTATAAGGGATTTT | |
| | GCCGATTTCGGCCTATTGGTTAAAAAATGAGCTGATTTA | |
| | ACAAAAATTTAACGCGAATTTTAACAAAATATTAACGC | |
| | TTACAATTTAGGTGGCACTTTTCGGGGAAATGTGCGCG | |
| | GAACCCCTATTTGTTTATTTTTCTAAATACATTCAAATA | |
| | TGTATCCGCTCATGAGACAATAACCCTGATAAATGCTTC | |
| | AATAATATTGAAAAAGGAAGAGTATGAGTATTCAACAT | |
| | TTCCGTGTCGCCCTTATTCCCTTTTTTGCGGCATTTTGCC | |
| | TTCCTGTTTTTGCTCACCCAGAAACGCTGGTGAAAGTAA | |
| | AAGATGCTGAAGATCAGTTGGGTGCACGAGTGGGTTAC | |
| | ATCGAACTGGATCTCAACAGCGGTAAGATCCTTGAGAG | |
| | TTTTCGCCCCGAAGAACGTTTTCCAATGATGAGCACTTT | |
| | TAAAGTTCTGCTATGTGGCGCGGTATTATCCCGTATTGA | |
| | CGCCGGGCAAGAGCAACTCGGTCGCCGCATACACTATT | |
| | CTCAGAATGACTTGGTTGAGTACTCACCAGTCACAGAA | |
| | AAGCATCTTACGGATGGCATGACAGTAAGAGAATTATG | |
| | CAGTGCTGCCATAACCATGAGTGATAACACTGCGGCCA | |
| | ACTTACTTCTGACAACGATCGGAGGACCGAAGGAGCTA | |
| | ACCGCTTTTTTGCACAACATGGGGGATCATGTAACTCGC | |
| | CTTGATCGTTGGGAACCGGAGCTGAATGAAGCCATACC | |
| | AAACGACGAGCGTGACACCACGATGCCTGTAGCAATGG | |
| | CAACAACGTTGCGCAAACTATTAACTGGCGAACTACTT | |
| | ACTCTAGCTTCCCGGCAACAATTAATAGACTGGATGGA | |
| | GGCGGATAAAGTTGCAGGACCACTTCTGCGCTCGGCCC | |
| | TTCCGGCTGGCTGGTTTATTGCTGATAAATCTGGAGCCG | |
| | GTGAGCGTGGGTCTCGCGGTATCATTGCAGCACTGGGG | |
| | CCAGATGGTAAGCCCTCCCGTATCGTAGTTATCTACACG | |
| | ACGGGGAGTCAGGCAACTATGGATGAACGAAATAGAC | |
| | AGATCGCTGAGATAGGTGCCTCACTGATTAAGCATTGG | |
| | TAACTGTCAGACCAAGTTTACTCATATATACTTTAGATT | |
| | GATTTAAAACTTCATTTTTAATTTAAAAGGATCTAGGTG | |
| | AAGATCCTTTTTGATAATCTCATGACCAAAATCCCTTAA | |
| | CGTGAGTTTTCGTTCCACTGAGCGTCAGACCCCGTAGAA | |
| | AAGATCAAAGGATCTTCTTGAGATCCTTTTTTTCTGCGC | |
| | GTAATCTGCTGCTTGCAAACAAAAAAACCACCGCTACC | |
| | AGCGGTGGTTTGTTTGCCGGATCAAGAGCTACCAACTCT | |
| | TTTTCCGAAGGTAACTGGCTTCAGCAGAGCGCAGATAC | |
| | CAAATACTGTTCTTCTAGTGTAGCCGTAGTTAGGCCACC | |
| | ACTTCAAGAACTCTGTAGCACCGCCTACATACCTCGCTC | |
| | TGCTAATCCTGTTACCAGTGGCTGCTGCCAGTGGCGATA | |
| | AGTCGTGTCTTACCGGGTTGGACTCAAGACGATAGTTA | |
| | CCGGATAAGGCGCAGCGGTCGGGCTGAACGGGGGGTTC | |
| | GTGCACACAGCCCAGCTTGGAGCGAACGACCTACACCG | |
| | AACTGAGATACCTACAGCGTGAGCTATGAGAAAGCGCC | |
| | ACGCTTCCCGAAGAGAGAAAGGCGGACAGGTATCCGGT | |
| | AAGCGGCAGGGTCGGAACAGGAGAGCGCACGAGGGAG | |
| | CTTCCAGGGGGAAACGCCTGGTATCTTTATAGTCCTGTC | |
| | GGGTTTCGCCACCTCTGACTTGAGCGTCGATTTTTGTGA | |
| | TGCTCGTCAGGGGGGCGGAGCCTATGGAAAAACGCCAG | |
| | CAACGCGGCCTTTTTACGGTTCCTGGCCTTTTGCTGGCC | |
| | TTTTGCTCACATGTTCTTTCCTGCGTTATCCCCTGATTCT | |
| | GTGGATAACCGTATTACCGCCTTTGAGTGAGCTGATACC | |
| | GCTCGCCGCAGCCGAACGACCGAGCGCAGCGAGTCAGT | |
| | GAGCGAGGAAGCGGAAGAGCGCCCAATACGCAAACCG | |
| | CCTCTCCCCGCGCGTTGGCCGATTCATTAATGCAGCTGG | |
| | CACGACAGGTTTCCCGACTGGAAAGCGGGCAGTGAGCG | |
| | CAACGCAATTAATGTGAGTTAGCTCACTCATTAGGCAC | |
| | CCCAGGCTTTACACTTTATGCTTCCGGCTCGTATGTTGT | |
| | GTGGAATTGTGAGCGGATAACAATTTCACACAGGAAAC | |
| | AGCTATGACCATGATTACGCCAAGCGCGCAATTAACCC | |
| | TCACTAAAGGGAACAAAAGCTGGAGCTGCAAGCTT | |

TABLE 7
Vector Description (Components added are bold-underlined)

| | | | | | |
|---|---|---|---|---|---|
| | | Size | | | |
| Name | Position | (bp) | Type | Description | Application notes |

VB220328-1304ynx Vector

| | | | | | |
|---|---|---|---|---|---|
| RSV | 1-229 | 229 | Promoter | Rous sarcoma | Strong promoter; drives |
| promoter | | | | virus | transcription of viral RNA |
| | | | | enhancer/promoter | in packaging cells |
| 5′ LTR-ΔU3 | 230-410 | 181 | LTR | Truncated HIV-1 | Allows transcription of |
| | | | | 5′ long terminal | viral RNA and its |
| | | | | repeat | packaging into virus. |
| Ψ | 521-565 | 45 | Miscellaneous | HIV-1 packaging | Allows packaging of viral |
| | | | | signal | RNA into virus. |
| RRE | 1075-1308 | 234 | Miscellaneous | HIV-1 Rev | Rev protein binding site |
| | | | | response element | that allows Rev dependent |
| | | | | | nuclear export of viral |
| | | | | | RNA during viral |
| | | | | | packaging. |
| cPPT | 1803-1920 | 118 | Miscellaneous | Central polypurine | Facilitates the nuclear |
| | | | | tract | import of HIV-1 DNA |
| | | | | | through a central DNA flap |
| SYN1 | 1950-2418 | 469 | Promoter | Tissue specificity: | |
| | | | Human | Brain. Cell type | |
| | | | synapsin I | specificity: | |
| | | | promoter | Mature neurons | |
| Kozak | 2443-2448 | 6 | Miscellaneous | Kozak translation | Facilitates translation |
| | | | | initiation sequence | initiation of ATG start |
| | | | | | codon downstream of the |
| | | | | | Kozak sequence. |
| rtOMP25C- | 2449-3069 | 621 | CDS | None | None |
| ter LentiWT | | | | | |
| IRES | 3094-3681 | 588 | Linker | Encephalomyo- | Recruits ribosome to |
| | | | | carditis virus | initiate translation |
| | | | | internal ribosome | internally on a transcript |
| | | | | entry site | independent of its 5′ end. |
| | | | | | Multiple proteins can be |
| | | | | | made from a polycistronic |
| | | | | | transcript containing |
| | | | | | multiple |
| EmGFP | 3682-4401 | 720 | CDS | Emerald green | Enhanced photostability |
| | | | | fluorescent | and brightness compared to |
| | | | | protein; variant of | its predecessor EGFP |
| | | | | EGFP generated | |
| | | | | by mutagenesis | |
| WPRE | 4431-5028 | 598 | Miscellaneous | Woodchuck | Enhances virus stability in |
| | | | | hepatitis virus | packaging cells, leading to |
| | | | | posttranscriptional | higher titer of packaged |
| | | | | regulatory element | virus; enhances higher |
| | | | | | expression of transgenes |
| 3′ LTR-ΔU3 | 5110-5344 | 235 | LTR | Truncated HIV-1 | Allows packaging of viral |
| | | | | 3′ long terminal | RNA into virus; self- |
| | | | | repeat | inactivates the 5′ LTR by a |
| | | | | | copying mechanism during |
| | | | | | viral genome integration; |
| | | | | | contains polyadenylation |
| | | | | | signal for transcription |
| | | | | | termination |
| SV40 early | 5417-5551 | 135 | PolyA signal | Simian virus 40 | Allows transcription |
| pA | | | | early | termination and |
| | | | | polyadenylation | polyadenylation of mRNA |
| | | | | signal | transcribed by Pol II RNA |
| | | | | | polymerase |
| Ampicillin | 6505-7365 | 861 | CDS | Ampicillin | Allows E. coli to be |
| | | | | resistance gene | resistant to ampicillin. |
| pUC ori | 7536-8124 | 589 | Rep_origin | pUC origin of | Facilitates plasmid |
| | | | | replication | replication in E. coli; |
| | | | | | regulates high-copy |
| | | | | | plasmid number (500-700) |

VB220329-1381pbx Vector

| | | | | | |
|---|---|---|---|---|---|
| RSV | 1-229 | 229 | Promoter Rous | Strong promoter; | |
| promoter | | | sarcoma virus | drives | |
| | | | enhancer/ | transcription of | |
| | | | promoter | viral RNA in | |
| | | | | packaging cells | |
| 5′ LTR-ΔU3 | 230-410 | 181 | LTR | Truncated HIV-1 | Allows transcription of |
| | | | | 5′ long terminal | viral RNA and its |
| | | | | repeat | packaging into virus |
| Ψ | 521-565 | 45 | Miscellaneous | HIV-1 packaging | Allows packaging of viral |
| | | | | signal | RNA into virus |
| RRE | 1075-1308 | 234 | Miscellaneous | HIV-1 Rev | Rev protein binding site |
| | | | | response element | that allows Rev-dependent |
| | | | | | nuclear export of viral |
| | | | | | RNA during viral |
| | | | | | packaging |
| cPPT | 1803-1920 | 118 | Miscellaneous | Central polypurine | Facilitates the nuclear |
| | | | | tract | import of HIV-1 cDNA |
| | | | | | through a central DNA flap |
| SYN1 | 1950-2418 | 469 | Promoter | Human synapsin I | Tissue specificity: Brain. |
| | | | | promoter | Cell type specificity: |
| | | | | | Mature neurons |
| Kozak | 2443-2448 | 6 | Miscellaneous | Kozak translation | Facilitates translation |
| | | | | initiation sequence | initiation of ATG start |
| | | | | | codon downstream of the |
| | | | | | Kozak sequence |
| hATAT1(2)rt | 2449-3561 | 1113 | CDS | None | None |
| OMP25C-ter | | | | | |
| LentiWT | | | | | |
| IRES | 3586-4173 | 588 | Linker | Encephalomyo- | Recruits ribosome to |
| | | | | carditis virus | initiate translation |
| | | | | internal ribosome | internally on a transcript |
| | | | | entry site | independent of its 5′ end. |
| | | | | | Multiple proteins can be |
| | | | | | made from a polycistronic |
| | | | | | transcript containing |
| | | | | | multiple ORFs separated |
| | | | | | by IRES |
| EmGFP | 4174-4893 | 720 | CDS | Emerald green | Enhanced photostability |
| | | | | fluorescent | and brightness compared to |
| | | | | protein; variant of | its predecessor EGFP |
| | | | | EGFP generated | |
| | | | | by mutagenesis | |
| WPRE | 4923-5520 | 598 | Miscellaneous | Woodchuck | Enhances virus stability in |
| | | | | hepatitis virus | packaging cells, leading to |
| | | | | posttranscriptional | higher titer of packaged |
| | | | | regulatory element | virus; enhances higher |
| | | | | | expression of transgenes |
| 3′ LTR-ΔU3 | 5602-5836 | 235 | LTR | Truncated HIV-1 | Allows packaging of viral |
| | | | | 3′ long terminal | RNA into virus; self- |
| | | | | repeat | inactivates the 5′ LTR by a |
| | | | | | copying mechanism during |
| | | | | | viral genome integration; |
| | | | | | contains polyadenylation |
| | | | | | signal for transcription |
| | | | | | termination |
| SV40 early | 5909-6043 | 135 | PolyA_signal | Simian virus 40 | Allows transcription |
| pA | | | | early | termination and |
| | | | | polyadenylation | polyadenylation of mRNA |
| | | | | signal | transcribed by Pol II RNA |
| | | | | | polymerase |
| Ampicillin | 6997-7857 | 861 | CDS | Ampicillin | Allows E. coli to be |
| | | | | resistance gene | resistant to ampicillin. |
| pUC ori | 8028-8616 | 589 | Rep_origin | pUC origin of | Facilitates plasmid |
| | | | | replication | replication in E. coli; |
| | | | | | regulates high-copy |
| | | | | | plasmid number (500-700) |

Altogether, these findings indicate that MFN2 dependent recruitment of ATAT1 to sites of mitochondrial contacts with MTs is conserved in sensory neurons and required for axonal integrity by maintaining normal levels of MT acetylation. Taking consideration of our functional data in MEF cells, and consistent with previous observations in cellular models of CMT2 caused by MFN2 mutations, these results also suggest that distal axonal degeneration caused by mutant MFN2 predominantly depends on loss of acetylated tubulin due to mutant MFN2-mediated high jacking of ATAT1, which affects mitochondrial motility and distribution, but not on the loss of fusion or functional mitochondria/ER tethering.

### 6.6. Conclusion

MFN2 mutations in CMT2A disrupt the fusion of mitochondria and compromise ER-mitochondrial interactions. However, while certain CMT2A mutant forms of MFN2 impair mitochondrial fusion and/or functional mitochondria/ER tethering, others do not affect either function [64], casting doubt on the implication of these MFN2 activities in the etiology of CMT2. The data presented above shows that MFN2 is a regulator of α-tubulin acetylation and MT dynamics, and that in MFN2 KO MEFs, rescuing α-tubulin acetylation levels by pharmacological inhibition of HDAC6 corrects defects in MT dynamics and mitochondrial motility and some MAM functions, but not MAM integrity or mitochondrial fusion.

In addition to modulation of HDAC6 protein levels, regulation of tubulin acetylation by MFN2 occurred through MFN2-mediated recruitment of ATAT1 to OMMs, an activity that is conserved in sensory neurons, critical for the induction of axonal degeneration by MFN2 loss of function, and impaired in MFN2 mutants associated with CMT2A. Interestingly, the binding of MFN2 to ATAT1 is dependent on the N-terminal catalytic domain of ATAT1. Conversely, both MFN2 R94W and T105M mutants disrupt the binding of ATAT1 with miro or kinesin-1, and an endogenous ATAT1/miro complex fails to form in cells deprived of MFN2 expression, suggesting that while ATAT1 binding to miro cannot depend on kinesin, the formation of a stable ATAT1/miro/kinesin-1 complex relies on WT MFN2. Based on these observations, it is believed that, in analogy to axonal vesicles, mitochondria contacts with microtubules (MTs) are hotspots of tubulin acetylation and that this function is impaired in CMT2 disease caused by MFN2 mutations.

The data also showed that mutant R94W or T105M MFN2 could not compensate for loss of MFN2 expression on either acetylated tubulin levels and axonal degeneration, and that expressing either MFN2 mutants is sufficient to induce axonal damage in WT DRG neurons. Thus, in CMT2A disease, mutant MFN2 likely drives axonal degeneration by disrupting the ability of mitochondria to release ATAT1 at specific sites on axonal MTs, leading to an imbalance in tubulin acetylation with consequent impairment of mitochondrial transport.

It was also determined that HDAC6 inhibition rescued the defect in MAM-dependent cholesterol and SM metabolism assayed by cellular lipidomic analysis but failed to normalize MAM-dependent phospholipid synthesis measured by incorporation of 3H-Ser into 3H-PtdSer (PS) and (H) 3H-PtdEtn (PE). Activation of MAM as a lipid raft, is strictly dependent on its enrichment on cholesterol and sphingomyelin levels, as well as the reorganization of ceramide and phospholipids with different saturation degrees. Thus, the above data indicate that the regulation of all these lipid classes is altered in MFN2 KO cells, and that the levels of cholesterol and SM are rescued by HDAC inhibition, but not those of specific phospholipids that are indispensable for the correct control of MAM functions. Indeed, HDAC6 inhibitors, and in particular TSA, were reported to modulate the expression of genes involved in cholesterol synthesis, uptake, and efflux and to restore cholesterol redistribution in models of neurodegenerative disorders such as Niemann-Pick's disease. Therefore, in the context of MFN2 ablation, TSA could rescue cholesterol efflux and SM turnover by this mechanism.

Thus, axonal degeneration caused by MFN2 loss of function mutations depends on loss of MFN2-dependent regulation of mitochondrial transport by interfering with ATAT1-dependent tubulin acetylation at sites of mitochondria and MT contact. This is consistent with a pathogenic role for disrupted mitochondrial transport in neuropathies and a key role for tubulin acetylation in mitochondrial dynamics. Taken together, these studies indicate that targeting HDAC or ATAT activity or expression provides a beneficial solution for restoring sensory neuron function by presenting an alternative therapeutic approach.

### INCORPORATION BY REFERENCE

All publications, patents, and patent applications mentioned herein are hereby incorporated by reference in their entirety as if each individual publication, patent, or patent application was specifically and individually indicated to be incorporated by reference. In case of conflict, the present application, including any definitions herein, will control.

Those skilled in the art will recognize or be able to ascertain using no more than routine experimentation, many equivalents to the specific embodiments described herein. Such equivalents are intended to be encompassed by the following claims.

What is claimed is:

1. A method of treating a subject suffering from a disease associated with peripheral neuropathy comprising:
administering to the subject, a composition comprising a nucleic acid encoding a fusion polypeptide comprising a nucleic acid sequence encoding a mitochondria targeting sequence operably linked to a nucleic acid sequence encoding a polypeptide sequence that increases levels of acetylated tubulin.

2. The method of claim 1, the nucleic acid encoding the fusion polypeptide further comprising a promoter sequence disposed upstream to the fusion polypeptide sequence.

3. The method of claim 2, wherein the promoter is a human synapsin I promoter (SYN1).

4. The method of claim 1, wherein the mitochondria targeting sequence is outer membrane protein 25 (OMP25).

5. The method of claim 1, wherein the polypeptide sequence that increases levels of acetylated tubulin is α-tubulin acetyltransferase 1 (ATAT1).

6. The method of claim 1, wherein the tubulin is α-tubulin.

7. The method of claim 1, wherein the mitochondria targeting sequence is fused to a C-terminus of the polypeptide sequence.

8. The method of claim 1, wherein the mitochondria targeting sequence is an OMP25 sequence, the polypeptide sequence that increases levels of acetylated tubulin is ATAT1, and the nucleic acid encoding the fusion polypeptide comprises the sequence set forth in SEQ ID NO.: 3.

9. The method of claim 1, the nucleic acid encoding the fusion polypeptide is further comprised in a viral vector.

10. The method of claim 9, wherein the viral vector is selected from the group consisting of adeno-associated virus (AAV), recombinant adenoviruses (rAV), and lentivirus (LV).

11. The method of claim 9, wherein the viral vector is a lentivirus.

12. The method of claim 11, wherein the viral vector comprising the nucleic acid encoding the fusion polypeptide comprises a sequence set forth in SEQ ID NO.: 4.

13. The method of claim 1, wherein the peripheral neuropathy is associated with diabetes, hypothyroidism, kidney disease, liver disease, cancer, Charcot-Marie-Tooth (CMT) disease, Guillain-Barré syndrome, autoimmune disease, rheumatoid arthritis, Lyme disease and lupus.

14. The method of claim 1, wherein the composition is administered to a peripheral nerve, a neuronal cell, or a peripheral blood mononuclear cell (PBMC).

15. A composition comprising:
a nucleic acid encoding a fusion polypeptide comprising a nucleic acid sequence encoding a mitochondria targeting sequence operably linked to a nucleic acid sequence encoding a polypeptide sequence that increases levels of acetylated tubulin.

16. The composition of claim 15, which is a pharmaceutical composition that further comprises a pharmaceutically acceptable carrier.

17. The composition of claim 15, the nucleic acid encoding the fusion polypeptide further comprising a promoter sequence disposed upstream to the fusion polypeptide sequence.

18. The composition of claim 17, wherein the promoter is human synapsin I promoter (SYN1).

19. The composition of claim 15, wherein the mitochondria targeting sequence is outer membrane protein 25 (OMP25).

20. The composition of claim 15, wherein the polypeptide that increases the levels of acetylated tubulin is α-tubulin acetyltransferase 1 (ATAT1).
